(19) United States
(12) Patent Application Publication
(10) Pub. No.: US 2026/0167626 A1
(43) Pub. Date: Jun. 18, 2026

(54) HETEROCYCLIC VAV1 DEGRADERS

(71) Applicant: INNOCARE PHARMA INC., Wilmington, DE (US)

(72) Inventors: Yingxiang GAO, Beijing (CN); Xiangyang CHEN, Beijing (CN)

(21) Appl. No.: 19/531,390

(22) Filed: Feb. 5, 2026

Related U.S. Application Data
(63) Continuation of application No. PCT/CN2025/092389, filed on Apr. 30, 2025.

(30) Foreign Application Priority Data
Sep. 2, 2024 (CN) 202411226410.8
Dec. 5, 2024 (CN) 202411784444.9

(51) Int. Cl. C07D 401/14; A61K 31/496; A61K 31/513; A61P 37/06

(52) U.S. Cl. CPC C07D 401/14; A61K 31/496; A61K 31/513; A61P 37/06

(57) ABSTRACT

The present invention relates to heterocyclic compounds or their pharmaceutically acceptable salts as VAV1 protein degraders. Specifically, the invention relates to compounds of Formula I and their pharmaceutically acceptable salts thereof. The invention also relates to methods for preparing the compounds of the present invention. The compounds of the present invention can be used for the treatment and/or prevention of VAV1-mediated diseases, including autoimmune and inflammatory diseases, and cancers.

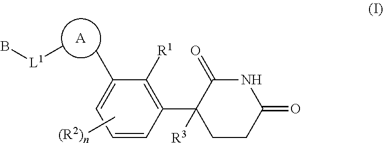

### CROSS-REFERENCE TO RELATED APPLICATION(S)

This application is a continuation of PCT/CN2025/092389, filed Apr. 30, 2025; which claims priority to Chinese Application Nos. 202411226410.8 filed Sep. 2, 2024, and 202411784444.9, filed Dec. 5, 2024. The contents of the above-identified applications are incorporated herein by reference in their entirety.

### TECHNICAL FIELD

The present invention relates to heterocyclic compounds of Formula I or their pharmaceutical acceptable salts thereof as VAV1 protein degraders. The invention also relates to methods for preparing compounds of Formula I. The invention further relates to the use of compounds of Formula I or their pharmaceutically acceptable salts thereof for the treatment and/or prevention of diseases mediated by VAV1, including autoimmune and inflammatory diseases, and

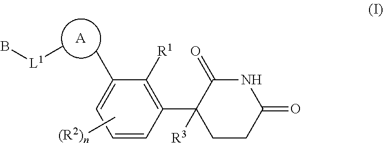

### BACKGROUND

VAV1 is a multi-domain protein with dual functions, acting as both a GEF (guanine nucleotide exchange factor) and a scaffolding protein involved in GEF-independent signaling pathways. The results from VAV1−/− mice indicate that VAV1 plays a critical role in the function of lymphocytes, such as T and B cells, as well as in antigen receptor signaling (Fischer, K. D. et al. Curr. Biol. 1998, 8, 554-562; Fujikawa, K. et al. J. Exp. Med. 2003, 198, 1595-1608). Therefore, targeted therapy against VAV1 has therapeutic potentials in the treatment of T and B cell-mediated autoimmune diseases, inflammation, and cancer.

However, due to the unique structure and function of VAV1, it is used to be considered as an undruggable target. Targeted protein degradation is a novel drug development strategy that facilitates the interaction between the ubiquitin-proteasome system (UPS) and disease-related proteins (referred to as “neo-substrates”) through small molecules (known as “molecular glues”), leading to the degradation of the neo-substrates. A clinically validated class of molecular glues is the glutarimide compounds, which bind to cereblon (CRBN) of the CRL4^{CRBN}-E3 ubiquitin ligase complex, inducing a change in the surface structure of the CRBN protein. This alteration promotes E3 ligase to recruit neo-substrates that are typically undruggable, leading to their ubiquitination by E2 and eventual recognition and degradation by 26S proteasome, thereby regulating the biological functions mediated by these neo-substrates. There is no clinical efficacy reported for degraders targeting VAV1.

The present invention relates to heterocyclic compounds according to Formula I and shows that compounds with such structures exhibit good activity in inducing VAV1 degradation.

### BRIEF DESCRIPTION OF THE DRAWINGS

FIG. 1 shows that daily oral dosing of Compound II-23 at 1 mg/kg significantly alleviated EAE progression with less clinical scores in the treatment group compared to those in the vehicle group.

FIG. 2 shows that daily oral dosing of Compound II-23 at 1 mg/kg did not have significant weight loss in the treatment group compared to those in the vehicle group.

### DETAILED DESCRIPTION OF THE INVENTION

### Definition

Unless otherwise stated, the following terms used in this application have the meanings described below.

“C_{x-y}” refers to the range of carbon atoms, where x and y are both integers. For example, C₃₋₈ cycloalkyl indicates a cycloalkyl group with 3-8 carbon atoms, that is, a cycloalkyl group with 3, 4, 5, 6, 7, or 8 carbon atoms. It should also be understood that “C₃₋₈” includes any sub-range within it, such as C₃₋₇, C₃₋₆, C₄₋₇, C₄₋₆, C₅₋₆, etc.

“Alkyl” refers to a saturated straight or branched hydrocarbon group containing 1 to 20 carbon atoms, such as 1 to 8 carbon atoms, 1 to 6 carbon atoms, or 1 to 4 carbon atoms. Non-limiting examples of alkyl include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, etc.

“Alkylene” refers to a divalent saturated hydrocarbon group containing 1 to 20 carbon atoms, such as a straight or branched chain with 1 to 6 carbon atoms, 1 to 4 carbon atoms, etc. Non-limiting examples of alkylene include —CH₂—, —CH(CH₃)—, —CH₂CH₂—, —CH₂CH₂CH₂—, —(CH₃)C(CH₃)—, —CH₂CH₂CH₂CH₂—, —CH₂CH(CH₃) CH₂—, etc.

“Cycloalkyl or cycloalkane” refers to a saturated cyclic hydrocarbon group containing 3 to 14 carbon ring atoms, which can be monocyclic, typically containing 3 to 8, 3 to 7, or 3 to 6 carbon ring atoms. Non-limiting examples of monocyclic cycloalkyl include cyclopropyl, cyclobutyl, cyclopentyl, cyclohexyl, cycloheptyl, etc. They can also be fused, bridged, or spirocyclic bicyclic or tricyclic, such as decahydronaphthyl, bicyclo[2.2.2]octane, spiro[3.3]heptane, etc.

“Cycloalkylene” refers to a divalent saturated carbocyclic group having 3 to 14 carbon ring atoms, for example, a structure as

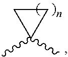

where n is an integer of 1 or greater than 1, typically n as 1 to 12. Non-limiting examples of cycloalkylene include cyclopropene, cyclobutene, cyclopentene, cyclohexene, etc.

“Heterocyclic or heterocycle” refers to a saturated or partially unsaturated monocyclic or polycyclic group, which include 3 to 20 ring atoms, for example, 3 to 14, 3 to 12, 3 to 10, 3 to 8, 3 to 6, or 5 to 6 ring atoms, where one or more ring atoms are selected from nitrogen, oxygen, or S(O)ₘ (where m is an integer from 0 to 2), and the remaining ring atoms are carbon. Preferably, it includes 3 to 12 ring atoms, 3 to 10 ring atoms, 4 to 7 ring atoms, 4 to 6 ring atoms, where 1 to 4 are heteroatoms, 1 to 3 are heteroatoms, or 1 to 2 are heteroatoms. Non-limiting examples of monocyclic heterocyclic include oxetanyl, tetrahydrofuranyl, tetrahydropyranyl, azetidinyl, pyrrolidinyl, piperidinyl, piperazinyl, morpholinyl, thiomorpholinyl, 1,4-oxazepanyl, pyrrolidinonyl, piperidinonyl, 1,1-dioxothiomorpholinyl, etc. Polycyclic heterocyclics include fused, bridged, or spiro polycyclic heterocycles, such as octahydrocyclopenta[c]pyrrole, octahydropyrrolo[1,2-a]pyrazine, 3,8-diazabicyclo[3.2.1]octane, 5-azaspiro[2.4]heptane, 2-oxa-7-azaspiro[3.5]nonane, etc.

“Aryl or aryl ring” refers to an aromatic monocyclic or fused polycyclic group containing 6 to 14 carbon atoms, preferably 6 to 10 members, such as phenyl and naphthyl, more preferably phenyl. The aryl ring can be fused to a heteroaryl, heterocyclic, or cycloalkyl ring, where the ring connected to the parent structure is the aryl ring. Non-limiting examples include:

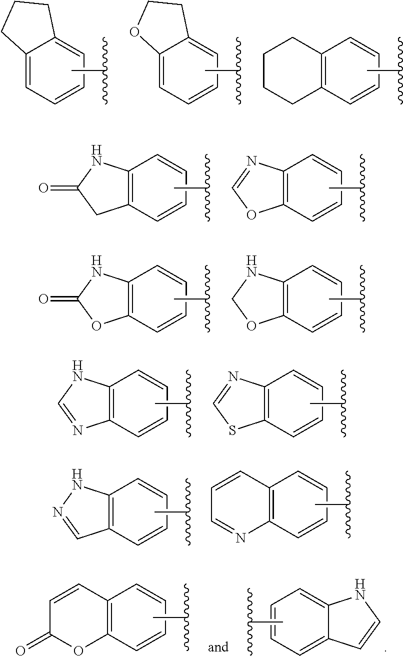

“Heteroaryl or heteroaryl ring” refers to a heteroaromatic system containing 5 to 14 ring atoms, where 1 to 4 ring atoms are selected from heteroatoms including oxygen, sulfur, and nitrogen. Heteroaryl is preferably 5 to 10-membered, more preferably 5-membered or 6-membered, such as furyl, thienyl, pyridyl, pyrrolyl, pyrimidinyl, pyrazinyl, pyrazolyl, imidazolyl, tetrazolyl, oxazolyl, isoxazolyl, thiazolyl, isothiazolyl, quinolinyl, isoquinolinyl, indolyl, isoindolyl, etc. The heteroaryl ring can be fused to an aryl, heterocyclic, or cycloalkyl ring. Non-limiting examples include:

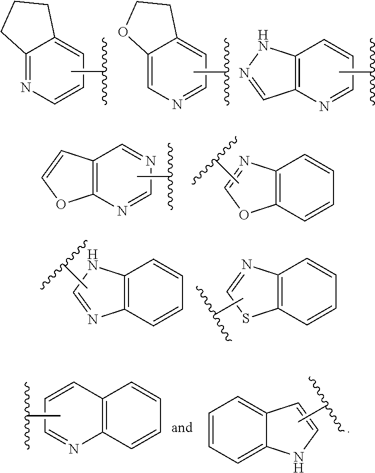

“Halogen” refers to fluorine, chlorine, bromine or iodine.

“Cyano” refers to —CN.

“Oxo” refers to ═O.

“Optional” means that the event or circumstance described subsequently may or may not occur. For example, the description “one or more hydrogens of phenyl are optionally substituted by halogen” includes scenarios where one or more hydrogens of phenyl are substituted by halogen and where none is substituted by halogen.

“Substitution” refers to one or more hydrogen atoms in a group, preferably 1 to 5, for example, 1 to 3 hydrogen atoms, being independently replaced by a corresponding number of substituents. Substituents are only located at possible chemical positions understood by those skilled in the art. For example, an amino or a hydroxyl group with free hydrogen may be unstable when connected to a carbon atom having an unsaturated bond (such as an alkene). Substituents include but are not limited to halogen, cyano, nitro, hydroxy, amino, oxo, —SF₅, C₁₋₄ alkyl, C₃₋₇ cycloalkyl, 4- to 7-membered heterocyclyl, phenyl, 5- to 6-membered heteroaryl, etc.

“Isomer” refers to a compound that has the same molecular formula but differs in the bonding positions or spatial arrangements of their atoms. Isomers with different spatial arrangements of atoms are called “stereoisomers”. Stereoisomers include optical isomers, geometric isomers, and conformational isomers.

The compounds of the present invention can exist in the form of optical isomers. Optical isomers include enantiomers and diastereomers. Enantiomers refer to two stereoisomers that are non-superimposable and are mirror images of each other. A racemic mixture or racemate refers to a chiral molecule mixture containing equal amounts of left-handed and right-handed enantiomers. Diastereomers refer to two stereoisomers that are non-superimposable and are not mirror images of each other. When the optical isomer is a single isomer and its absolute configuration is determined, it is referred to as “R” or “S” isomer based on the configuration of the substituents on the chiral atom. When the absolute configuration is not determined, it is referred to as the (+) or (−) isomer based on its measured optical rotation value. The methods for preparing and separating optical isomers are known in the art.

The compounds of the present invention may also have geometric isomers with substituents distributed differently around carbon-carbon double bonds, carbon-nitrogen double bonds, cycloalkyl, or heterocyclic group. Substituents around carbon-carbon double bonds or carbon-nitrogen bonds are designated as Z or E configurations, while substituents around cycloalkyl or heterocyclic group are designated as cis or trans configurations.

The compounds of the present invention may also exhibit tautomerism, such as keto-enol tautomerism.

The present invention includes any tautomeric or stereoisomeric forms and their mixtures and is not limited to any one tautomeric or stereoisomeric form used in the naming or chemical structural formula of the compound.

“Isotopes” refer to all isotopes of atoms that appear in the compounds of this invention. Isotopes include those atoms having the same atomic number but different mass numbers. Examples of isotopes suitable for incorporation into the compounds of this invention are hydrogen, carbon, nitrogen, oxygen, phosphorus, sulfur, fluorine, and chlorine, specifically but not limited to ²H (D), ³H, ¹³C, ¹⁴C, ¹⁵N, ¹⁷O, ¹⁸O, ³¹P, ³²P, ³⁵S, ¹⁸F and ³⁶Cl. The isotopically labeled compounds of this invention can generally be prepared by conventional techniques known to those skilled in the art or by using appropriately isotopically labeled reagents in place of non-isotopically labeled reagents in methods similar to those described in the examples. Such compounds have various potential uses, such as standards and reagents in determining biological activity. In terms of stable isotopes, such as deuterium ²H (D), ¹³C, and ¹⁵N, compounds containing such isotopes have the potential to alter biological, pharmacological or pharmacokinetic properties. Deuterium ²H (D) is a preferred isotope of this invention, for example, hydrogens in methyl, methylene, or methyne can be replaced by deuterium.

The compounds of the present invention can be administered in the form of prodrugs. “Prodrug” refers to a derivative that is converted into biologically active compound of the present invention under physiological conditions in vivo, for example, through oxidation, reduction, hydrolysis (each of which occurs with or without the participation of enzymes). Examples of prodrugs include the following compounds of the present invention in which an amino is acylated, alkylated or phosphorylated, for example, eicosanoyl amino, alanyl amino and pivaloyloxymethyl amino, or a hydroxyl is acylated, alkylated, phosphorylated or converted into borate, for example, acetoxy, palmitoyloxy, pivaloyloxy, succinyloxy, fumaroyloxy and alanyloxy, or a carboxyl is esterified or amidated, or a thiol is conjugated with a carrier molecule that selectively delivers the drug to the target and/or to the cytosol of cells. These compounds can be prepared from the compounds of the present invention by known methods.

“Pharmaceutically acceptable salts” refer to salts made from the compounds of the present invention containing one or more acidic and basic groups with pharmaceutically acceptable bases or acids, including inorganic bases or acids and organic bases or acids. Therefore, compounds of the present invention containing acidic groups can exist in form of salts, such as alkali metal salts, alkaline earth metal salts, or ammonium salts. More specific examples of such salts include sodium salts, potassium salts, calcium salts, magnesium salts, or salts with ammonia or organic amines, such as ethylamine, ethanolamine, triethanolamine, or amino acids. Compounds of the present invention containing basic groups can exist in form of inorganic or organic acid salts. Examples of suitable acids include hydrochloric acid, hydrobromic acid, phosphoric acid, sulfuric acid, nitric acid, methanesulfonic acid, p-toluenesulfonic acid, naphthalenedisulfonic acid, oxalic acid, acetic acid, tartaric acid, lactic acid, salicylic acid, benzoic acid, formic acid, propionic acid, pivalic acid, malonic acid, succinic acid, pimelic acid, fumaric acid, maleic acid, malic acid, sulfamic acid, phenylpropionic acid, gluconic acid, ascorbic acid, isonicotinic acid, citric acid, adipic acid, and other acids known to those skilled in the art. If compound of the present invention contains both acidic and basic groups in the molecule, the present invention includes zwitterions or internal salts in addition to the mentioned salt forms. Each salt can be obtained by conventional methods known to those skilled in the art, such as by mixing the compound with an organic or inorganic acid or base in a solvent or dispersant, or by anion or cation exchange with other salts.

“Pharmaceutical composition” refers to a composition containing one or more compounds of the present invention or their pharmaceutically acceptable salts, stable isotope derivatives, isomers, prodrugs and mixtures thereof, as well as other components, such as pharmaceutically acceptable carriers and excipients.

When “compounds” are mentioned in the present invention, all compound forms are included, such as pharmaceutically acceptable salts, stable isotope derivatives, isomers, prodrugs, and mixtures thereof.

“Therapeutically effective amount” refers to the amount of the compound of the present invention that can effectively degrade VAV1, for the treatment or prevention of diseases mediated by VAV1.

“Patients” refer to mammals, preferably humans.

The present invention relates to compounds as VAV1 degraders, having a structure of Formula I, or their pharmaceutically acceptable salts, stable isotope derivatives, isomers and prodrugs thereof:

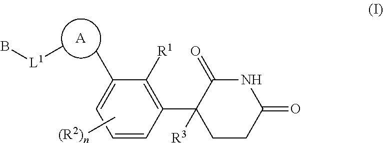

wherein:
Ring A is 4- to 10-membered heterocycle or C₃₋₁₀ cycloalkane, where one or more hydrogens of the heterocycle and cycloalkane are optionally substituted by R⁴;
B is 5- to 10-membered heteroaryl, C₆₋₁₀ aryl, C₁₋₆ alkyl, C₃₋₈ cycloalkyl or 4- to 10-membered heterocyclyl, where one or more hydrogens of the heteroaryl, aryl, alkyl, cycloalkyl and heterocyclyl are optionally substituted by R⁵;
L¹ is bond, —C₁₋₆ alkylene-, —C₃₋₆ cycloalkylene-, —C(O)—, —O—, —S(O)₀₋₂—, —NR′—, *—OC₁-6 alkylene-**, *—S(O)₀₋₂C₁₋₆ alkylene-**, *—NR′C₁₋₆ alkylene-**, *—C₁₋₆ alkylene-O—**, *—C₁₋₆ alkylene-S(O)₀₋₂—**, *—C₁₋₆ alkylene-NR′—**, *—NR′C(O)—** or *—C(O)NR′—**, where * indicates L¹ connected to ring A, ** indicates L¹ connected to B, and one or more hydrogens of the alkylene and cycloalkylene are optionally substituted by D, halogen or C₁₋₆ alkyl;
R¹ is halogen, H, cyano, C₁₋₆ alkyl, C₃₋₈ cycloalkyl or —OC₁₋₆ alkyl, where one or more hydrogens of the alkyl are optionally substituted by D or halogen;
Each R² is independently H, halogen, cyano, —OR′, —NR′R″, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OR′ or C₁₋₆ alkyl;
R³ is H, D, halogen, C₁₋₆ alkyl or fluorinated C₁₋₆ alkyl;
R⁴ and R⁵ are each independently C₁₋₆ alkyl, oxo, D, halogen, cyano, —OR′, —NR′R″, —COOH, —C(O)NR′R″, C₃₋₈ cycloalkyl, C₃₋₈ cycloalkylene or 4- to 8-membered heterocyclyl, where one or more hydrogens of the alkyl, cycloalkyl, cycloalkylene and heterocyclyl are optionally substituted by D, halogen, —OR′ or C₁₋₆ alkyl;
R′ and R″ are each independently H, C₁₋₆ alkyl, C₃₋₈ cycloalkyl or 4- to 8-membered heterocyclyl, where one or more hydrogens of the alkyl, cycloalkyl and heterocyclyl are optionally substituted by D, halogen, —OC₁₋₆ alkyl or C₁₋₆ alkyl; and
n is an integer from 1 to 3.

In one embodiment, ring A is

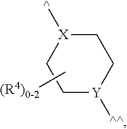

where \hat{ }indicates the bond connected to L¹, \hat{ }\hat{ }indicates the bond connected to the benzene ring, X and Y are each independently CH or N, and each R⁴ is independently C₁₋₆ alkyl, oxo, D, halogen, cyano, —OR′, fluorinated C₁₋₆ alkyl, C₃₋₈ cycloalkyl or C₃₋₈ cycloalkylene.

In one embodiment, ring A is piperazine, where one or more hydrogens of the piperazine are optionally substituted by C₁₋₆ alkyl, C₃₋₈ cycloalkylene or oxo.

In one embodiment, B is 5- to 6-membered heteroaryl, phenyl or 5- to 6-membered heterocyclyl, where one or more hydrogens of the heteroaryl, phenyl and heterocyclyl are optionally substituted by oxo, halogen, cyano, —OR′, —NR′R″, —COOH, —C(O)NR′R″, C₁₋₆ alkyl or C₃₋₈ cycloalkyl, where one or more hydrogens of the alkyl are further optionally substituted by D, For —OR′.

In one embodiment, B is 6-membered heteroaryl, where one or more hydrogens of the heteroaryl are optionally substituted by oxo, —NR′R″, —C(O)NR′R″, C₁₋₆ alkyl or C₃₋₈ cycloalkyl, where one or more hydrogens of the alkyl are further optionally substituted by D or F.

In one embodiment, L¹ is bond or —C₁₋₆ alkylene-.

In one embodiment, L¹ is bond.

In one embodiment, R¹ is halogen.

In one embodiment, R² is H.

In one embodiment, R³ is H.

In one embodiment, R′ and R″ are each H.

In some embodiments, the compounds of the present invention have Formula II:

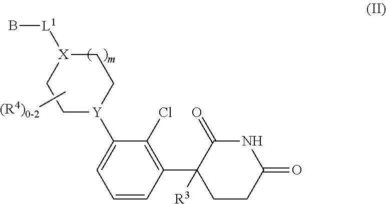

wherein:
X and Y are each independently CH or N;
B is 5- to 6-membered heteroaryl, phenyl or 4- to 10-membered heterocyclyl, where one or more hydrogens of the heteroaryl, phenyl and heterocyclyl are optionally substituted by R⁵;
L¹ is bond or —C₁₋₆ alkylene-;
R³ is H, D, halogen or C₁₋₆ alkyl;
R⁴ is C₁₋₆ alkyl, C₃₋₈ cycloalkylene, oxo, D or halogen, where one or more hydrogens of the alkyl are optionally substituted by D or halogen;
R⁵ is C₁₋₆ alkyl, oxo, halogen, cyano, —OR′, —NR′R″, —COOH, —C(O)NR′R″ or C₃₋₈ cycloalkyl, where one or more hydrogens of the alkyl are optionally substituted by D or F;
R′ and R″ are each independently H or C₁₋₆ alkyl; and
m is an integer from 0 to 2.

In one embodiment, X and Y are N.

In one embodiment, B is 6-membered heteroaryl or 6-membered heterocyclyl.

In one embodiment, L¹ is bond.

In one embodiment, R³ is H.

In one embodiment, R⁴ is C₁₋₆ alkyl, C₃₋₈ cycloalkylene or H.

In one embodiment, m is 1.

In some embodiments, the compounds of the present invention have Formula III:

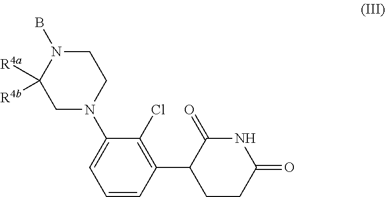

wherein:
B is 6-membered heteroaryl, where one or more hydrogens of the heteroaryl are optionally substituted by oxo, —NR′R″, —C(O)NR′R″, C₁₋₆ alkyl or C₃₋₈ cycloalkyl, where one or more hydrogens of the alkyl are further optionally substituted by D or F;
R^{4a} and R^{4b} are each independently H, C₁₋₆ alkyl, D, halogen, or together with the carbon atom attached to form a C₃₋₈ cycloalkyl ring; and
R′ and R″ are each independently H or C₁₋₆ alkyl.

The invention also relates to the following compounds, and their pharmaceutically acceptable salts, stable isotope derivatives, isomers, prodrugs and mixtures thereof:

| Compound # | Compound Structure and Nomenclature |
|---|---|
| II-1 | 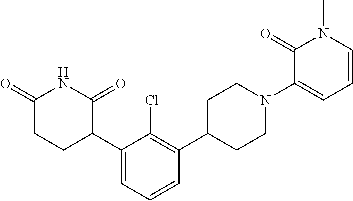 [SMILES: Cn1cccc(N2CCC(c3cccc(C4CCC(=O)NC4=O)c3Cl)CC2)c1=O] |
| | 3-(2-chloro-3-(1-(1-methyl-2-oxo-1,2-dihydropyridin-3-yl)piperidin-4- |
| | yl)phenyl)piperidine-2,6-dione |
| II-2 | 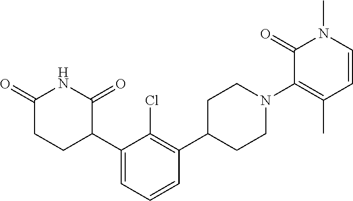 [SMILES: Cc1ccn(C)c(=O)c1N1CCC(c2cccc(C3CCC(=O)NC3=O)c2Cl)CC1] |
| | 3-(2-chloro-3-(1-(1,4-dimethyl-2-oxo-1,2-dihydropyridin-3-yl)piperidin- |
| | 4-yl)phenyl)piperidine-2,6-dione |
| II-3 | 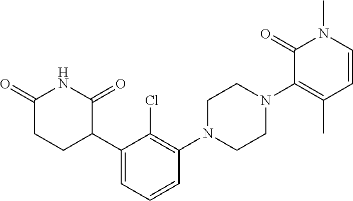 [SMILES: Cc1ccn(C)c(=O)c1N1CCN(c2cccc(C3CCC(=O)NC3=O)c2Cl)CC1] |
| | 3-(2-chloro-3-(4-(1,4-dimethyl-2-oxo-1,2-dihydropyridin-3-yl)piperazin- |
| | 1-yl)phenyl)piperidine-2,6-dione |
| II-4 | 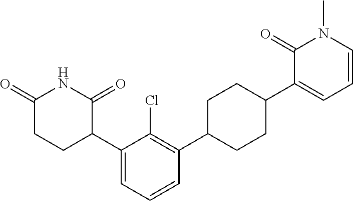 [SMILES: Cn1cccc(C2CCC(c3cccc(C4CCC(=O)NC4=O)c3Cl)CC2)c1=O] |
| | 3-(2-chloro-3-(4-(1-methyl-2-oxo-1,2-dihydropyridin-3- |
| | yl)cyclohexyl)phenyl)piperidine-2,6-dione |
| II-5 | 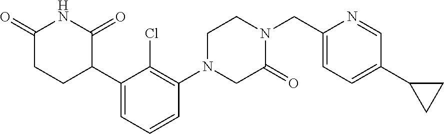 [SMILES: O=C1CCC(c2cccc(N3CCN(Cc4ccc(C5CC5)cn4)C(=O)C3)c2Cl)C(=O)N1] |
| | 3-(2-chloro-3-(4-((5-cyclopropylpyridin-2-yl)methyl)-3-oxopiperazin-1- |
| | yl)phenyl)piperidine-2,6-dione |
| II-6 | 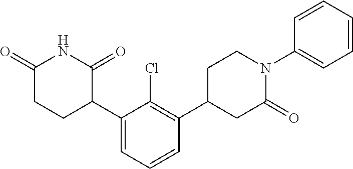 [SMILES: O=C1CCC(c2cccc(C3CCN(c4ccccc4)C(=O)C3)c2Cl)C(=O)N1] |
| | 3-(2-chloro-3-(2-oxo-1-phenylpiperidin-4-yl)phenyl)piperidine-2,6-dione |
| II-7 | 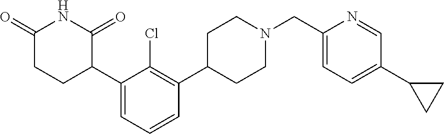 [SMILES: O=C1CCC(c2cccc(C3CCN(Cc4ccc(C5CC5)cn4)CC3)c2Cl)C(=O)N1] |
| | 3-(2-chloro-3-(1-((5-cyclopropylpyridin-2-yl)methyl)piperidin-4- |
| | yl)phenyl)piperidine-2,6-dione |
| II-8 | 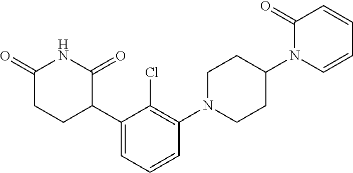 [SMILES: O=C1CCC(c2cccc(N3CCC(n4ccccc4=O)CC3)c2Cl)C(=O)N1] |
| | 3-(2-chloro-3-(4-(2-oxopyridin-1(2H)-yl)piperidin-1-yl)phenyl)piperidine- |
| | 2,6-dione |
| II-9 | 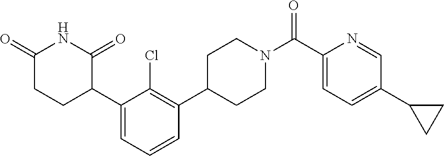 [SMILES: O=C1CCC(c2cccc(C3CCN(C(=O)c4ccc(C5CC5)cn4)CC3)c2Cl)C(=O)N1] |
| | 3-(2-chloro-3-(1-(5-cyclopropylpicolinoyl)piperidin-4- |
| | yl)phenyl)piperidine-2,6-dione |
| II-10 | 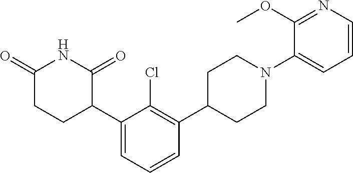 [SMILES: COc1ncccc1N1CCC(c2cccc(C3CCC(=O)NC3=O)c2Cl)CC1] |
| | 3-(2-chloro-3-(1-(2-methoxypyridin-3-yl)piperidin-4- |
| | yl)phenyl)piperidine-2,6-dione |
| II-11 | 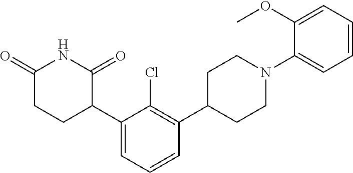 [SMILES: COc1ccccc1N1CCC(c2cccc(C3CCC(=O)NC3=O)c2Cl)CC1] |
| | 3-(2-chloro-3-(1-(2-methoxyphenyl)piperidin-4- |
| | yl)phenyl)piperidine-2,6-dione |
| II-12 | 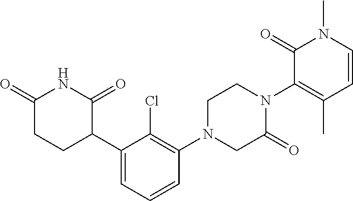 [SMILES: Cc1ccn(C)c(=O)c1N1CCN(c2cccc(C3CCC(=O)NC3=O)c2Cl)CC1=O] |
| | 3-(2-chloro-3-(4-(1,4-dimethyl-2-oxo-1,2-dihydropyridin-3-yl)-3- |
| | oxopiperazin-1-yl)phenyl)piperidine-2,6-dione |
| II-13 | 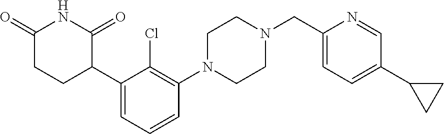 [SMILES: O=C1CCC(c2cccc(N3CCN(Cc4ccc(C5CC5)cn4)CC3)c2Cl)C(=O)N1] |
| | 3-(2-chloro-3-(4-((5-cyclopropylpyridin-2-yl)methyl)piperazin-1- |
| | yl)phenyl)piperidine-2,6-dione |
| II-14 | 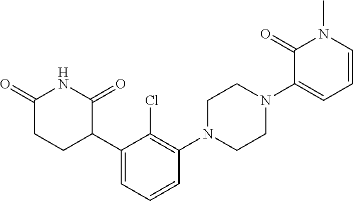 [SMILES: Cn1cccc(N2CCN(c3cccc(C4CCC(=O)NC4=O)c3Cl)CC2)c1=O] |
| | 3-(2-chloro-3-(4-(1-methyl-2-oxo-1,2-dihydropyridin-3-yl) |
| | piperazin-1-yl)phenyl)piperidine-2,6-dione |
| II-15 | 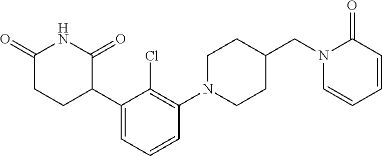 [SMILES: O=C1CCC(c2cccc(N3CCC(Cn4ccccc4=O)CC3)c2Cl)C(=O)N1] |
| | 3-(2-chloro-3-(4-((2-oxopyridin-1(2H)-yl)methyl)piperidin-1- |
| | yl)phenyl)piperidine-2,6-dione |
| II-16 | 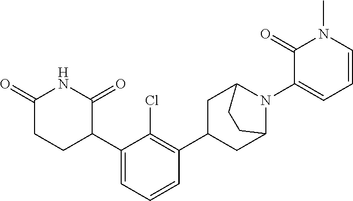 [SMILES: Cn1cccc(N2C3CCC2CC(c2cccc(C4CCC(=O)NC4=O)c2Cl)C3)c1=O] |
| | 3-(2-chloro-3-(8-(1-methyl-2-oxo-1,2-dihydropyridin-3-yl)-8- |
| | azabicyclo[3.2.1]octan-3-yl)phenyl)piperidine-2,6-dione |
| II-17 | 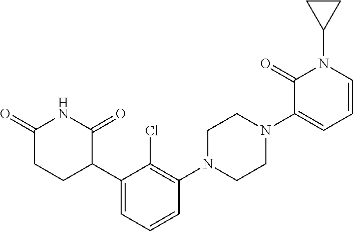 [SMILES: O=C1CCC(c2cccc(N3CCN(c4cccn(C5CC5)c4=O)CC3)c2Cl)C(=O)N1] |
| | 3-(2-chloro-3-(4-(1-cyclopropyl-2-oxo-1,2-dihydropyridin-3-yl)piperazin- |
| | 1-yl)phenyl)piperidine-2,6-dione |
| II-18 | 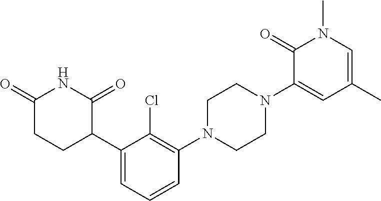 [SMILES: Cc1cc(N2CCN(c3cccc(C4CCC(=O)NC4=O)c3Cl)CC2)c(=O)n(C)c1] |
| | 3-(2-chloro-3-(4-(1,5-dimethyl-2-oxo-1,2-dihydropyridin-3-yl)piperazin- |
| | 1-yl)phenyl)piperidine-2,6-dione |
| II-19 | 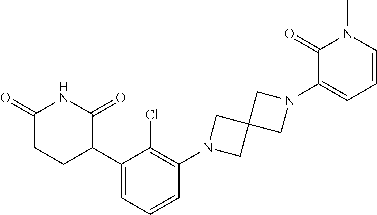 [SMILES: Cn1cccc(N2CC3(CN(c4cccc(C5CCC(=O)NC5=O)c4Cl)C3)C2)c1=O] |
| | 3-(2-chloro-3-(6-(1-methyl-2-oxo-1,2-dihydropyridin-3-yl)-2,6- |
| | diazaspiro[3.3]heptan-2-yl)phenyl)piperidine-2,6-dione |
| II-20 | 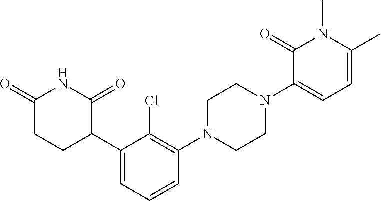 [SMILES: Cc1ccc(N2CCN(c3cccc(C4CCC(=O)NC4=O)c3Cl)CC2)c(=O)n1C] |
| | 3-(2-chloro-3-(4-(1,6-dimethyl-2-oxo-1,2-dihydropyridin-3-yl)piperazin- |
| | 1-yl)phenyl)piperidine-2,6-dione |
| II-21 | 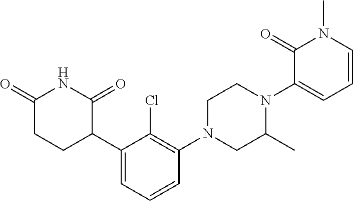 [SMILES: CC1CN(c2cccc(C3CCC(=O)NC3=O)c2Cl)CCN1c1cccn(C)c1=O] |
| | 3-(2-chloro-3-(3-methyl-4-(1-methyl-2-oxo-1,2-dihydropyridin-3- |
| | yl)piperazin-1-yl)phenyl)piperidine-2,6-dione |
| II-22 | 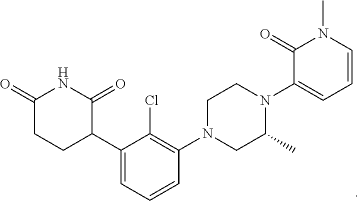 [SMILES: C[C@@H]1CN(c2cccc(C3CCC(=O)NC3=O)c2Cl)CCN1c1cccn(C)c1=O] |
| | 3-(2-chloro-3-((R)-3-methyl-4-(1-methyl-2-oxo-1,2-dihydropyridin-3- |
| | yl)piperazin-1-yl)phenyl)piperidine-2,6-dione |
| II-23 | 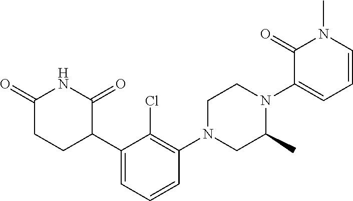 [SMILES: C[C@H]1CN(c2cccc(C3CCC(=O)NC3=O)c2Cl)CCN1c1cccn(C)c1=O] |
| | 3-(2-chloro-3-((S)-3-methyl-4-(1-methyl-2-oxo-1,2-dihydropyridin-3- |
| | yl)piperazin-1-yl)phenyl)piperidine-2,6-dione |
| II-24 | 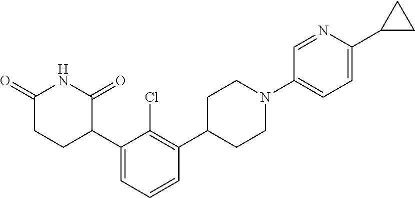 [SMILES: O=C1CCC(c2cccc(C3CCN(c4ccc(C5CC5)nc4)CC3)c2Cl)C(=O)N1] |
| | 3-(2-chloro-3-(1-(6-cyclopropylpyridin-3-yl)piperidin-4- |
| | yl)phenyl)piperidine-2,6-dione |
| II-25 | 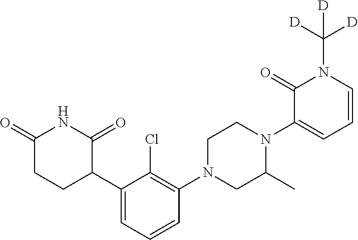 [SMILES: [2H]C([2H])([2H])n1cccc(N2CCN(c3cccc(C4CCC(=O)NC4=O)c3Cl)CC2C)c1=O] |
| | 3-(2-chloro-3-(3-methyl-4-(1-(methyl-d₃)-2-oxo-1,2-dihydropyridin-3- |
| | yl)piperazin-1-yl)phenyl)piperidine-2,6-dione |
| II-26 | 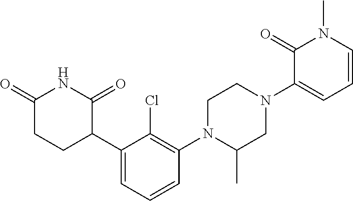 [SMILES: CC1CN(c2cccn(C)c2=O)CCN1c1cccc(C2CCC(=O)NC2=O)c1Cl] |
| | 3-(2-chloro-3-(2-methyl-4-(1-methyl-2-oxo-1,2-dihydropyridin-3- |
| | yl)piperazin-1-yl)phenyl)piperidine-2,6-dione |
| II-27 | 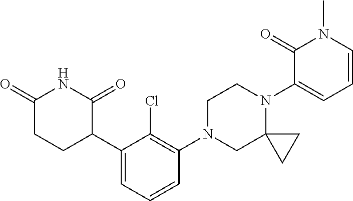 [SMILES: Cn1cccc(N2CCN(c3cccc(C4CCC(=O)NC4=O)c3Cl)CC23CC3)c1=O] |
| | 3-(2-chloro-3-(4-(1-methyl-2-oxo-1,2-dihydropyridin-3-yl)-4,7- |
| | diazaspiro[2.5]octan-7-yl)phenyl)piperidine-2,6-dione |
| II-28 | 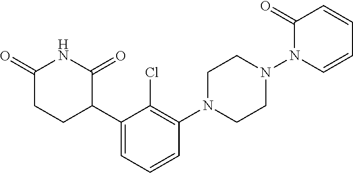 [SMILES: O=C1CCC(c2cccc(N3CCN(n4ccccc4=O)CC3)c2Cl)C(=O)N1] |
| | 3-(2-chloro-3-(4-(2-oxopyridin-1(2H)-yl)piperazin-1- |
| | yl)phenyl)piperidine-2,6-dione |
| II-29 | 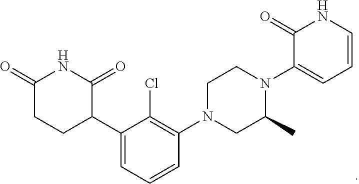 [SMILES: C[C@H]1CN(c2cccc(C3CCC(=O)NC3=O)c2Cl)CCN1c1ccc[nH]c1=O] |
| | 3-(2-chloro-3-((S)-3-methyl-4-(2-oxo-1,2-dihydropyridin-3-yl)piperazin- |
| | 1-yl)phenyl)piperidine-2,6-dione |
| II-30 | 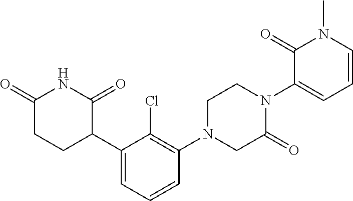 [SMILES: Cn1cccc(N2CCN(c3cccc(C4CCC(=O)NC4=O)c3Cl)CC2=O)c1=O] |
| | 3-(2-chloro-3-(4-(1-methyl-2-oxo-1,2-dihydropyridin-3-yl)-3- |
| | oxopiperazin-1-yl)phenyl)piperidine-2,6-dione |
| II-31 | 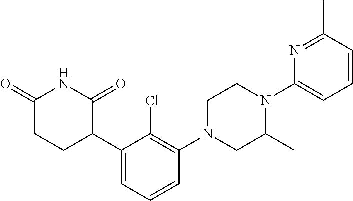 [SMILES: Cc1cccc(N2CCN(c3cccc(C4CCC(=O)NC4=O)c3Cl)CC2C)n1] |
| | 3-(2-chloro-3-(3-methyl-4-(6-methylpyridin-2-yl)piperazin-1- |
| | yl)phenyl)piperidine-2,6-dione |
| II-32 | 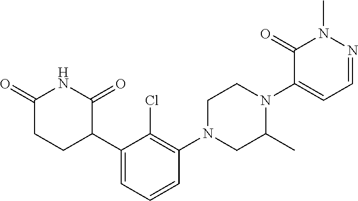 [SMILES: CC1CN(c2cccc(C3CCC(=O)NC3=O)c2Cl)CCN1c1ccnn(C)c1=O] |
| | 3-(2-chloro-3-(3-methyl-4-(2-methyl-3-oxo-2,3-dihydropyridazin-4- |
| | yl)piperazin-1-yl)phenyl)piperidine-2,6-dione |
| II-33 | 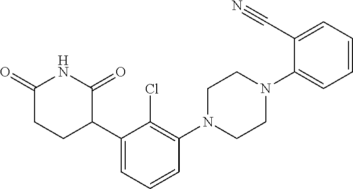 [SMILES: N#Cc1ccccc1N1CCN(c2cccc(C3CCC(=O)NC3=O)c2Cl)CC1] |
| | 2-(4-(2-chloro-3-(2,6-dioxopiperidin-3-yl)phenyl)piperazin-1- |
| | yl)benzonitrile |
| II-34 | 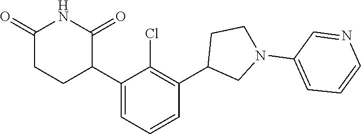 [SMILES: O=C1CCC(c2cccc(C3CCN(c4cccnc4)C3)c2Cl)C(=O)N1] |
| | 3-(2-chloro-3-(1-(pyridin-3-yl)pyrrolidin-3-yl)phenyl)piperidine-2,6-dione |
| II-35 | 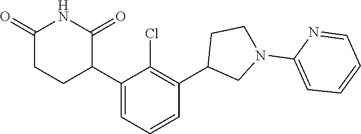 [SMILES: O=C1CCC(c2cccc(C3CCN(c4ccccn4)C3)c2Cl)C(=O)N1] |
| | 3-(2-chloro-3-(1-(pyridin-2-yl)pyrrolidin-3-yl)phenyl)piperidine-2,6-dione |
| II-36 | 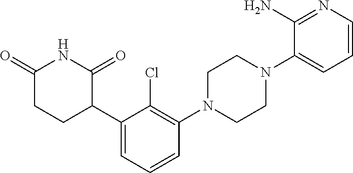 [SMILES: Nc1ncccc1N1CCN(c2cccc(C3CCC(=O)NC3=O)c2Cl)CC1] |
| | 3-(3-(4-(2-aminopyridin-3-yl)piperazin-1-yl)-2-chlorophenyl)piperidine- |
| | 2,6-dione |
| II-37 | 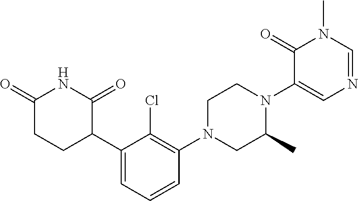 [SMILES: C[C@H]1CN(c2cccc(C3CCC(=O)NC3=O)c2Cl)CCN1c1cncn(C)c1=O] |
| | 3-(2-chloro-3-((S)-3-methyl-4-(1-methyl-6-oxo-1,6-dihydropyrimidin-5- |
| | yl)piperazin-1-yl)phenyl)piperidine-2,6-dione |
| II-38 | 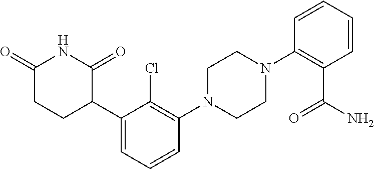 [SMILES: NC(=O)c1ccccc1N1CCN(c2cccc(C3CCC(=O)NC3=O)c2Cl)CC1] |
| | 2-(4-(2-chloro-3-(2,6-dioxopiperidin-3-yl)phenyl)piperazin-1- |
| | yl)benzamide |
| II-39 | 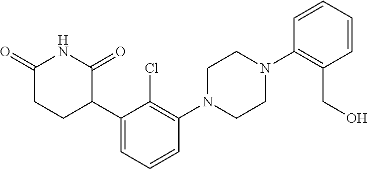 [SMILES: O=C1CCC(c2cccc(N3CCN(c4ccccc4CO)CC3)c2Cl)C(=O)N1] |
| | 3-(2-chloro-3-(4-(2-(hydroxymethyl)phenyl)piperazin-1- |
| | yl)phenyl)piperidine-2,6-dione |
| II-40 | 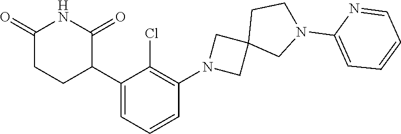 [SMILES: O=C1CCC(c2cccc(N3CC4(CCN(c5ccccn5)C4)C3)c2Cl)C(=O)N1] |
| | 3-(2-chloro-3-(6-(pyridin-2-yl)-2,6-diazaspiro[3.4]octan-2- |
| | yl)phenyl)piperidine-2,6-dione |
| II-41 | 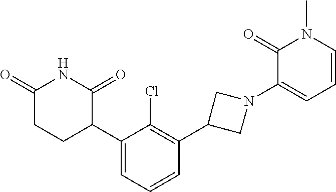 [SMILES: Cn1cccc(N2CC(c3cccc(C4CCC(=O)NC4=O)c3Cl)C2)c1=O] |
| | 3-(2-chloro-3-(1-(1-methyl-2-oxo-1,2-dihydropyridin-3-yl)azetidin-3- |
| | yl)phenyl)piperidine-2,6-dione |
| II-42 | 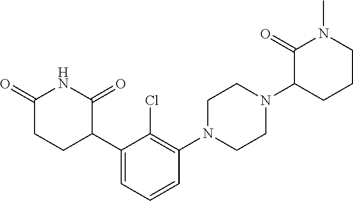 [SMILES: CN1CCCC(N2CCN(c3cccc(C4CCC(=O)NC4=O)c3Cl)CC2)C1=O] |
| | 3-(2-chloro-3-(4-(1-methyl-2-oxopiperidin-3-yl)piperazin-1- |
| | yl)phenyl)piperidine-2,6-dione |
| II-43 | 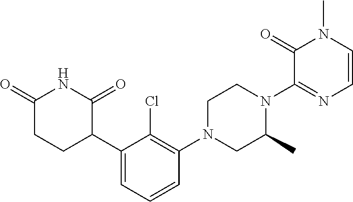 [SMILES: C[C@H]1CN(c2cccc(C3CCC(=O)NC3=O)c2Cl)CCN1c1nccn(C)c1=O] |
| | 3-(2-chloro-3-((S)-3-methyl-4-(4-methyl-3-oxo-3,4-dihydropyrazin-2- |
| | yl)piperazin-1-yl)phenyl)piperidine-2,6-dione |
| II-44 | 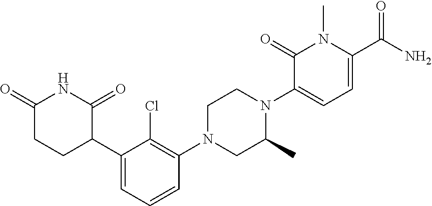 [SMILES: C[C@H]1CN(c2cccc(C3CCC(=O)NC3=O)c2Cl)CCN1c1ccc(C(N)=O)n(C)c1=O] |
| | 5-((2S)-4-(2-chloro-3-(2,6-dioxopiperidin-3-yl)phenyl)-2- |
| | methylpiperazin-1-yl)-1-methyl-6-oxo-1,6-dihydropyridine-2- |
| | carboxamide |
| II-45 | 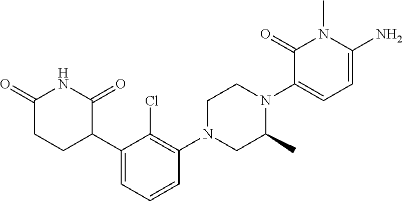 [SMILES: C[C@H]1CN(c2cccc(C3CCC(=O)NC3=O)c2Cl)CCN1c1ccc(N)n(C)c1=O] |
| | 3-(3-((S)-4-(6-amino-1-methyl-2-oxo-1,2-dihydropyridin-3-yl)-3- |
| | methylpiperazin-1-yl)-2-chlorophenyl)piperidine-2,6-dione |

The compounds of the present invention can effectively induce the degradation of VAV1, with a DC₅₀ preferably less than 100 nM. The compounds of the present invention can also effectively inhibit the activation of IL-2 induced by CD3/CD28, with an IC₅₀ preferably less than 100 nM. The compounds of the present invention can effectively alleviate disease progression in the mouse EAE model.

The invention also relates to a pharmaceutical composition, which comprises a compound of Formula I or a pharmaceutically acceptable salt, a stable isotope derivative, an isomer and a prodrug thereof, along with one or more pharmaceutically acceptable carriers or excipients. The pharmaceutical composition can induce the degradation of VAV1, affecting its biological function for treating or preventing diseases mediated by VAV1.

The present invention provides a method for treating or preventing diseases mediated by VAV1, which comprises administering to a patient in need a therapeutically effective amount of a compound of Formula I or a pharmaceutically acceptable salt, stable isotope derivative, isomer, prodrug and pharmaceutical composition thereof. The diseases include but are not limited to autoimmune-related diseases (such as rheumatoid arthritis, systemic lupus erythematosus, psoriasis, multiple sclerosis, inflammatory bowel disease, neurodegenerative diseases, diabetes, etc.) and cancers (such as B or T-cell related hematologic tumors, solid tumors, etc.).

The invention also provides the use of a compound of Formula I or the pharmaceutically acceptable salt, stable isotope derivative, isomer, or pharmaceutical composition thereof for the treatment or prevention of diseases mediated by VAV1, where diseases mediated by VAV1 include but are not limited to autoimmune-related diseases (such as rheumatoid arthritis, systemic lupus erythematosus, psoriasis, multiple sclerosis, inflammatory bowel disease, neurodegenerative diseases, diabetes, etc.) and cancer (such as B or T-cell related hematological tumors, solid tumors, etc.).

The invention further provides a compound of Formula I or the pharmaceutically acceptable salt, stable isotope derivative, isomer or pharmaceutical composition thereof for use in the preparation of a medicine, where the medicine is for the treatment or prevention of diseases mediated by VAV1, including but not limited to autoimmune-related diseases (such as rheumatoid arthritis, systemic lupus erythematosus, psoriasis, multiple sclerosis, inflammatory bowel disease, neurodegenerative diseases, diabetes, etc.) and cancer (such as B or T-cell related hematological tumors, solid tumors, etc.).

According to the present invention, the pharmaceuticals can be in any dosage form, including but not limited to tablets, capsules, solutions, lyophilized preparations and injectables.

The pharmaceutical formulation of the present invention can be administered in a form of dosage units containing a predetermined amount of active ingredient. Depending on the disease being treated, the method of administration, as well as age, weight and condition of the patient, such a unit may contain 0.1 to 500 mg of a compound of the present invention. Furthermore, the pharmaceutical formulation can be prepared using methods well known in the pharmaceutical field, such as by formulating the active ingredient with one or more excipients or one or more adjuvants.

The pharmaceutical formulation of the present invention is suitable for administration by any appropriate method, such as oral (including buccal or sublingual), rectal, nasal, topical (including buccal, sublingual or transdermal) or parenteral (including subcutaneous, intramuscular, intravenous or intradermal) administration.

The invention also provides a method for preparing the compounds. The compounds can be synthesized using synthetic methods known to those skilled in the art. The products obtained from each step of the reaction are isolated by separation techniques known in the field. The starting materials and chemical reagents used for the synthesis can be conventionally made according to the literature which can be searched from SciFinder or purchased.

The heterocyclic compounds shown in Formula I of the present invention can be synthesized by Buchwald or Ullmann coupling reaction from halide T-1 and T-2. T can be further derivatized to give other target compounds according to conventional chemical reactions.

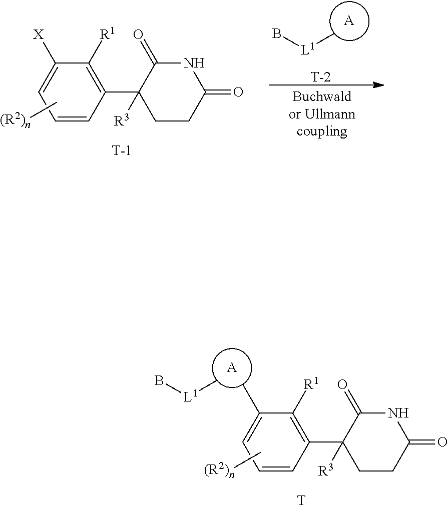

T can also be synthesized according to the following route: a) T-1 and T-3 undergoes Buchwald or Ullmann coupling to give T-6; or T-1 and T-4 undergoes Suzuki coupling to give T-5, which is hydrogenated to give T-6; b) T-6 undergoes deprotection to generate T-7; c) T-7 and T-8 undergoes appropriate transformation to generate T.

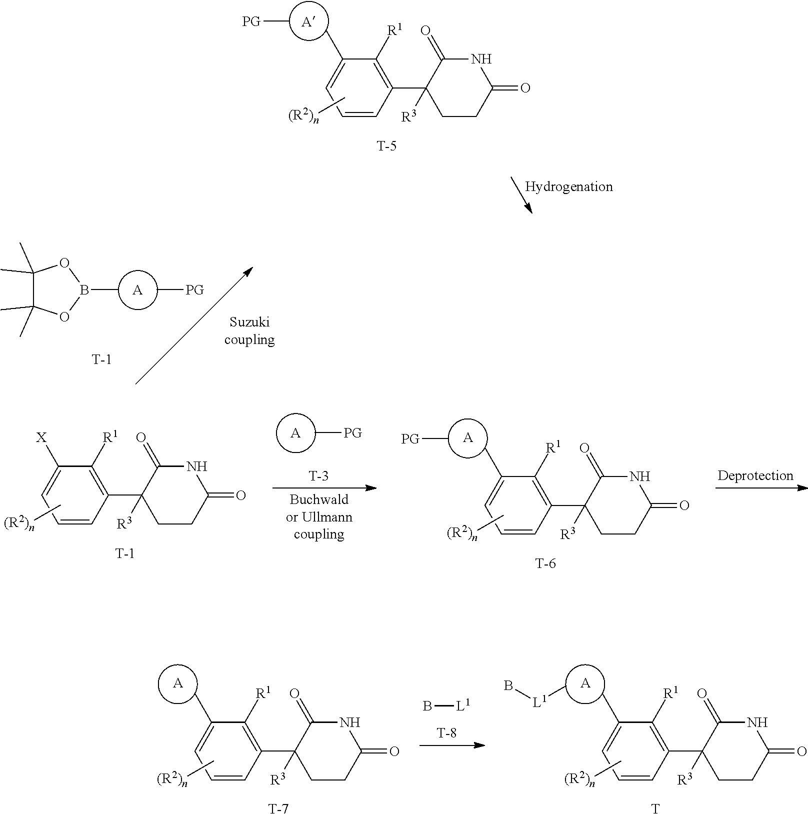

T can also be synthesized by Buchwald or Ullmann coupling reaction from halide T-9 and T-2, followed by cyclization under acidic condition.

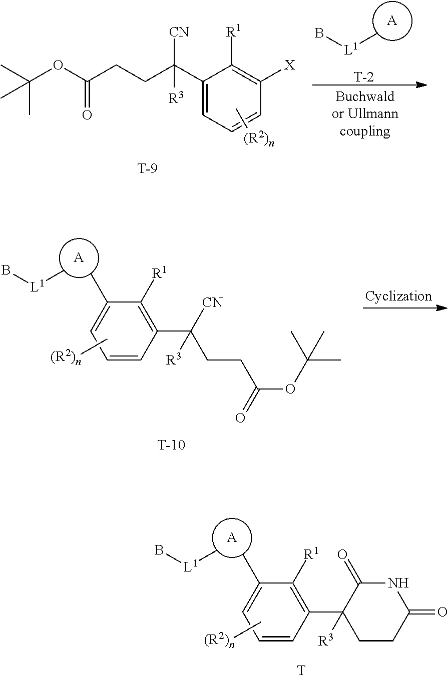

T can also be synthesized according to the following route: a) T-9 and T-3 undergoes Buchwald or Ullmann coupling to give T-12; or T-9 and T-4 undergoes Suzuki coupling to give T-11, which is hydrogenated to give T-12; b) T-12 undergoes deprotection to generate T-13; c) T-13 and T-8 undergoes appropriate transformation to generate T.

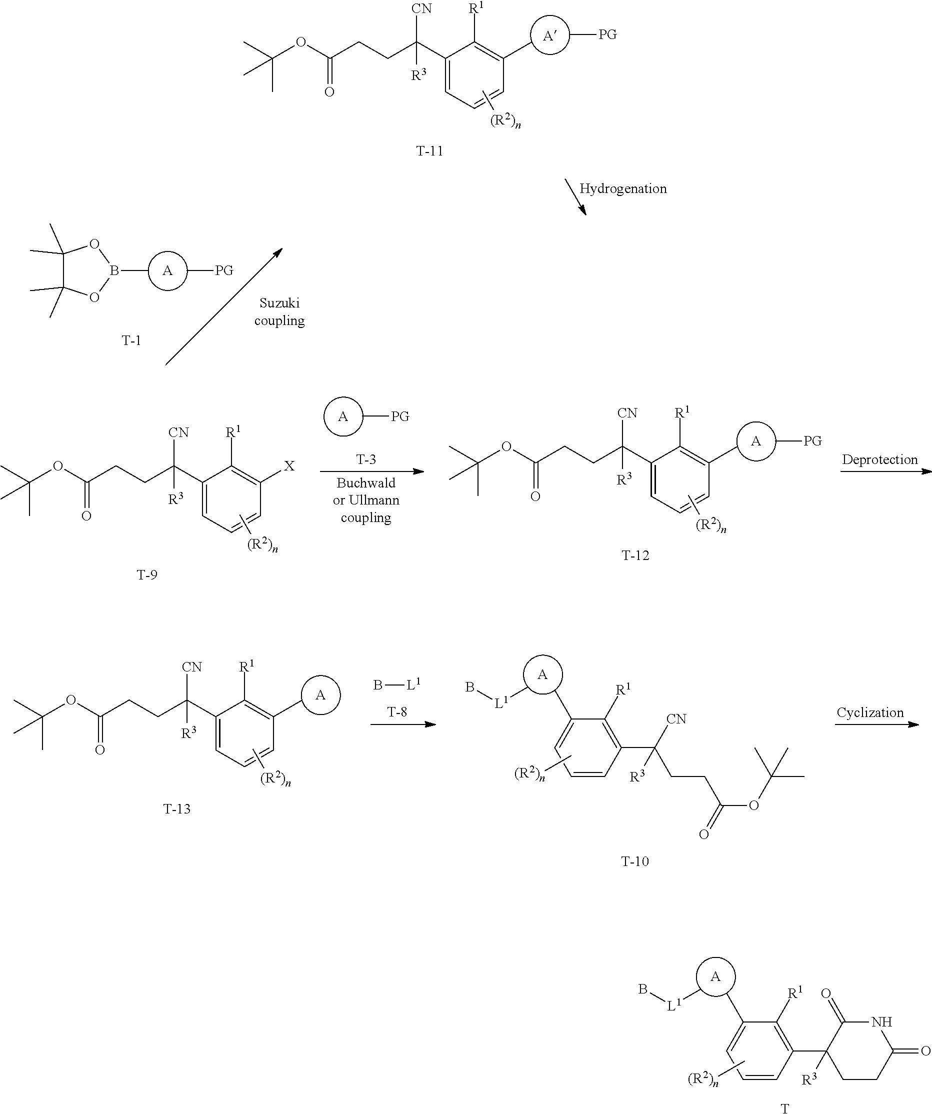

Halide T-1 and T-9 can be synthesized according to the following route: a) T-9a is brominated by NBS, then substituted with cyano to give T-9b; b) T-9b can undergo further substitution reactions to generate T-9c; c) T-9c undergoes addition with tert-butyl acrylate in the presence of a base to give T-9; d) T-9 undergoes cyclization under acid condition to give T-1.

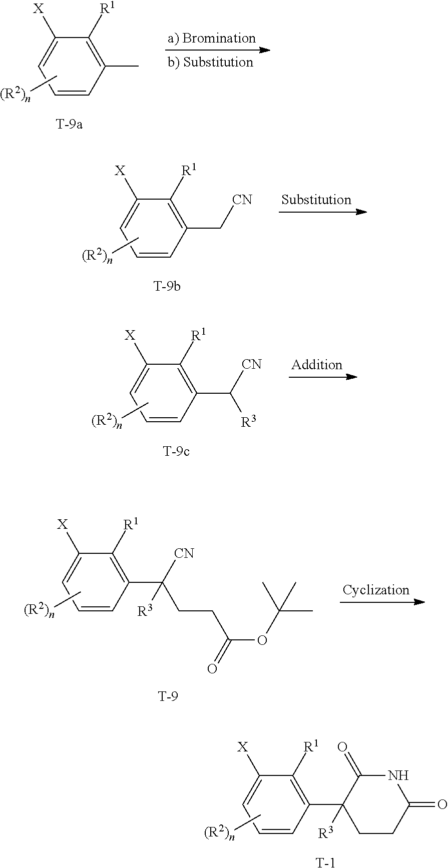

Other intermediates can be purchased or synthesized by routine transformations from commercially available starting materials.

The following examples further illustrate the present invention. These examples are intended solely to illustrate the invention and should not be considered as limiting the scope of the invention.

### EXAMPLES

The starting materials of the present invention were synthesized according to methods known in the art, or purchased from chemical companies such as ABCR GmbH & Co. KG, Acros Organics, Aldrich Chemical Company, Accela ChemBio Inc., Beijing Coupling, Bide Pharmatech, Zesheng Technology, etc.

The structures of the compounds of the present invention were determined by nuclear magnetic resonance (NMR) and/or mass spectrometry (MS). The NMR measurement used a Bruker ASCEND-400 NMR spectrometer, with a solvent, such as deuterated dimethyl sulfoxide (DMSO-d₆), deuterated chloroform (CDCl₃), deuterated methanol (CD₃OD), and tetramethylsilane (TMS) as the internal standard. Chemical shift was given in a unit of 10⁻⁶ (ppm). MS measurement used an Agilent SQD (ESI) mass spectrometer (Agilent 6120).

HPLC used an Agilent 1260 DAD high-performance liquid chromatography system (Poroshell 120 EC-C18, 50×3.0 mm, 2.7 μm column) or a Waters Arc high-performance liquid chromatography system (Sunfire C18, 150×4.6 mm, 5 μm column).

Unless otherwise stated, the reaction was run at room temperature (20° C.-30° C.).

Unless otherwise stated, the reaction was carried out under an argon or nitrogen atmosphere. An argon or nitrogen atmosphere refers to a reaction flask connected to an approximately 1-L volume balloon filled with argon or nitrogen.

A hydrogen atmosphere refers to the reaction flask being evacuated and then refilled with hydrogen gas (repeated 3 times), followed by connecting a hydrogen balloon with a volume of approximately 1 L.

The microwave reaction used a CEM Discover-SP type microwave reactor.

The reaction was monitored using an Agilent's liquid chromatography-mass spectrometry (LC-MS) system (1260/6120) and thin-layer chromatography (TLC) with a thickness of the silica gel plate being 0.15˜0.2 mm (Qingdao Haiyang GF254).

The compound purification was carried out by column chromatography or thin-layer chromatography, where column chromatography used 200˜300 mesh silica gel from Qingdao Haiyang and thin-layer chromatography used GF254 silica gel plates with a thickness of 0.4˜0.5 mm from Qingdao Haiyang.

Column chromatography or thin-layer chromatography eluent solvent systems typically included a) dichloromethane and methanol, b) ethyl acetate and hexane, or as shown in examples. The ratio of solvents was adjusted according to the polarity of the compound and further adjusted by addition of a small amount of triethylamine or other acidic or basic reagents.

The purification of the compounds also used a mass spectrometry-guided automated preparation system from Waters (mass spectrometer: SQD2). Depending on the polarity of the compound, an appropriate acetonitrile/water gradient (containing 0.1% trifluoroacetic acid or formic acid, or 0.05% ammonia) was used to elute the reverse-phase high-pressure column (XBridge-C18, 19×150 mm, 5 μm) at a flow rate of 20 mL/min.

The abbreviation DMSO refers to dimethyl sulfoxide.

The abbreviation DMF refers to N,N-dimethylformamide.

The abbreviation THF refers to tetrahydrofuran.

The abbreviation DMA refers to N,N-dimethylacetamide.

The abbreviation TBAF refers to tetrabutylammonium fluoride.

The abbreviation Pd₂(dba)₃ refers to tris(dibenzylideneacetone) dipalladium.

The abbreviation Pd(dppf)Cl₂ refers to [1,1′-bis(diphenylphosphino) ferrocene]dichloropalladium.

The abbreviation NBS refers to N-bromosuccinimide.

The abbreviation AIBN refers to azodiisobutyronitrile.

The abbreviation TMSCN refers to trimethylsilyl cyanide.

The abbreviation DIPEA refers to N,N-diisopropylethylamine.

The abbreviation DMAP refers to 4-dimethylaminopyridine.

The abbreviation TsCl refers to p-toluenesulfonyl chloride.

The abbreviation DBU refers to 1,8-diazabicyclo[5.4.0]undec-7-ene.

The abbreviation SEM-Cl refers to 2-(trimethylsilyl) ethoxymethyl chloride.

The abbreviation Pd(PPh₃)₄ refers to tetrakis(triphenylphosphine) palladium.

The abbreviation Xantphos refers to 4,5-bis(diphenylphosphino)-9,9-dimethylxanthene.

The abbreviation RuPhos refers to 2-dicyclohexylphosphino-2′,6′-diisopropoxy-1,1′-biphenyl.

The abbreviation RuPhos-Pd-G3 refers to palladium (II) (2-dicyclohexylphosphino-2′,6′-diisopropoxy-1,1′-biphenyl) (2-amino-1,1′-biphenyl-2-yl) methanesulfonate.

The abbreviation PhNTf₂ refers to N,N-bis(trifluoromethylsulfonyl) aniline.

The abbreviation CDI refers to N,N′-carbonyldiimidazole.

The abbreviation BINAP refers to 2,2′-bis(diphenylphosphino)-1,1′-dinaphthalene.

The abbreviation HATU refers to O-(7-azabenzotriazol-1-yl)-N,N,N′,N′-tetramethyluronium hexafluorophosphate.

The abbreviation EA refers to ethyl acetate.

The abbreviation MTBE refers to methyl tert-butyl ether.

The abbreviation prep-HPLC refers to preparative reversed-phase high-performance liquid chromatography.

### Example 1. Synthesis of Intermediates

### Intermediate A: 3-(3-bromo-2-chlorophenyl) piperidine-2,6-dione

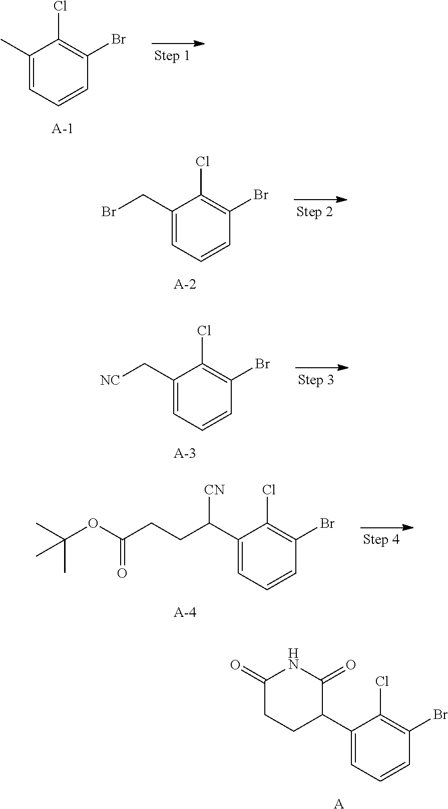

### Step 1

### 1-Bromo-3-(bromomethyl)-2-chlorobenzene (A-2)

To a mixture of 1-bromo-2-chloro-3-methylbenzene A-1 (2.9 g, 14 mmol), NBS (2.6 g, 15 mmol) and carbon tetrachloride (28 mL) was added AIBN (0.23 g, 1.4 mmol). The reaction mixture was stirred under reflux for 30 minutes. The mixture was filtered, and the filtrate was concentrated under reduced pressure. The residue was purified by silica gel column chromatography (petroleum ether/ethyl acetate=100/0 to 0/100) to give the target product A-2 (2.4 g, 60%).

### Step 2

### 2-(3-Bromo-2-chlorophenyl) acetonitrile (A-3)

To a mixture of A-2 (2.4 g, 8.4 mmol) and dichloromethane (25 mL) at 0° C. were added TMSCN (1.6 mL, 12.6 mmol) and a THF solution of TBAF (1.0 M, 13 mL, 13 mmol). The reaction mixture was stirred at room temperature for 1.5 hours. The mixture was quenched with water and extracted with dichloromethane. The organic phase was concentrated under reduced pressure, and the residue was purified by silica gel column chromatography (petroleum ether/ethyl acetate=100/0 to 0/100) to give the target product A-3 (1.7 g, 86%).

### Step 3

### tert-Butyl 4-(3-bromo-2-chlorophenyl)-4-cyanobutanoate (A-4)

To a mixture of A-3 (1.66 g, 7.25 mmol), tert-butyl acrylate (1.1 mL, 7.25 mmol), and THF (22 mL) at 0° C. was added sodium methoxide (78 mg, 1.45 mmol). The reaction mixture was stirred at room temperature for 2 hours, quenched with water and extracted with dichloromethane. The organic phase was concentrated under reduced pressure, and the residue was purified by silica gel column chromatography (petroleum ether/ethyl acetate=100/0 to 0/100) to give the target product A-4 (2.2 g, 96%).

MS m/z (ESI): 358.1 [M+H⁺]

### Step 4

### 3-(3-Bromo-2-chlorophenyl) piperidine-2,6-dione (A)

To a mixture of A-4 (2.5 g, 7.0 mmol) and acetic acid (20 mL) was added concentrated sulfuric acid (2 mL), and the mixture was stirred at 90° C. for 3 hours. After cooling to room temperature, ice water was added to the mixture. The resulting mixture was filtered, and the filter cake was washed with water and dried to give the target product A (1.7 g, 82%).

MS m/z (ESI): 301.9 [M+H⁺]

### Intermediate B: 3-(2-chloro-3-(4,4,5,5-tetramethyl-1,3,2-dioxaborolan-2-yl)phenyl) piperidine-2,6-dione

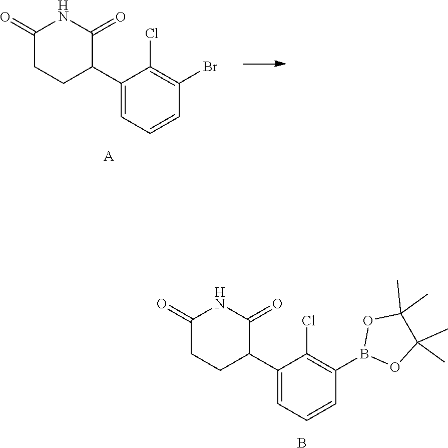

To a solution of A (500 mg, 1.7 mmol) in 1,4-dioxane (10 mL) were added 4,4,4′,4′,5,5,5′,5′-octamethyl-2,2′-bi (1,3,2-dioxaborolane) (633 mg, 2.5 mmol), Pd(dppf)Cl₂ (122 mg, 0.17 mmol) and potassium acetate (326 mg, 3.3 mmol). The mixture was stirred at 90° C. for 2 hours. After cooling to room temperature, the mixture was concentrated under reduced pressure. The residue was purified by prep-HPLC to give the target product B (290 mg, 50%).

MS m/z (ESI): 350.2 [M+H⁺]

### Example 2

### 3-(2-chloro-3-(1-(1-methyl-2-oxo-1,2-dihydropyridin-3-yl) piperidin-4-yl)phenyl) piperidine-2,6-dione (Compound II-1)

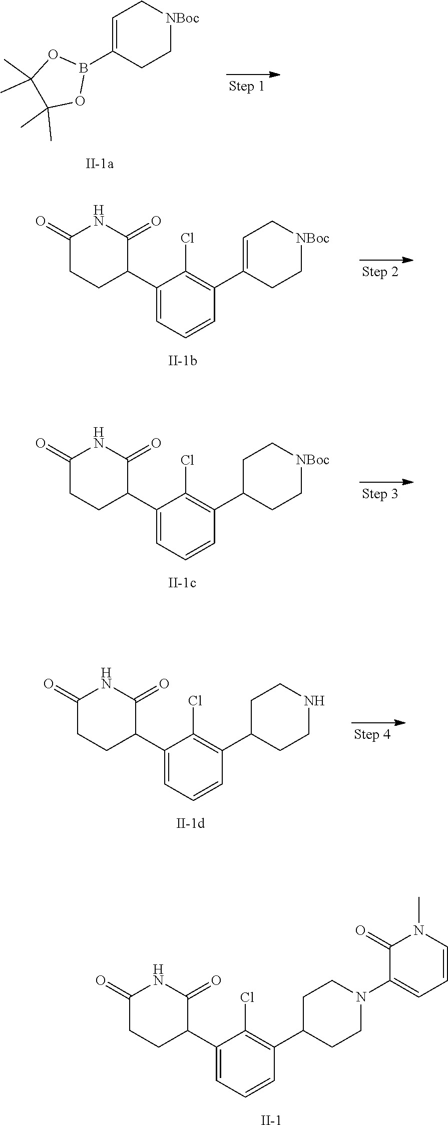

### Step 1

### tert-Butyl 4-(2-chloro-3-(2,6-dioxopiperidin-3-yl)phenyl)-3,6-dihydropyridine-1 (2H)-carboxylate (II-1b)

To a mixture of A (0.30 g, 0.99 mmol), potassium phosphate (0.63 g, 3.0 mmol), II-1a (0.46 g, 1.5 mmol) and 1,4-dioxane (10 mL) was added Pd(dppf)Cl₂ (0.08 g, 0.10 mmol), and the reaction mixture was stirred at 100° C. for 16 hours. Water was added and the resulting mixture was extracted with ethyl acetate. The organic phase was concentrated under reduced pressure, and the residue was purified by silica gel column chromatography (petroleum ether/ethyl acetate=100/0 to 0/100) to give the target product II-1b (280 mg, 70%).

### Step 2

### tert-Butyl 4-(2-chloro-3-(2,6-dioxopiperidin-3-yl)phenyl) piperidine-1-carboxylate (II-1c)

To a solution of II-1b (0.18 g, 0.44 mmol) in methanol (10 mL) was added platinum (IV) oxide (60 mg), and the reaction mixture was stirred at room temperature under a hydrogen atmosphere for 3 hours. The mixture was filtered, and the filter cake was washed with methanol. The filtrate was concentrated under reduced pressure to give the target product II-1c (crude, 180 mg). The product was used directly in the next step without further purification.

MS m/z (ESI): 307.1 [M+H+−100]

### Step 3

### 3-(2-Chloro-3-(piperidin-4-yl)phenyl) piperidine-2,6-dione (II-1d)

To a solution of II-1c (0.18 g, crude product) in dichloromethane (4 mL) was added a solution of hydrogen chloride in 1,4-dioxane (4 M, 4 mL). The reaction mixture was stirred at room temperature for 1 hour and concentrated under reduced pressure to give the target product II-1d as a hydrochloride salt (crude, 180 mg). The product was used directly in the next step without further purification.

MS m/z (ESI): 307.1 [M+H⁺]

### Step 4

### 3-(2-Chloro-3-(1-(1-methyl-2-oxo-1,2-dihydropyridin-3-yl) piperidin-4-yl)phenyl) piperidine-2,6-dione (II-1)

To a mixture of II-1d (50 mg, crude), 3-bromo-1-methylpyridin-2 (1H)-one (36 mg, 0.19 mmol), potassium carbonate (67 mg, 0.49 mmol), L-proline (4 mg, 0.03 mmol) and DMSO (2 mL) was added copper (I) iodide (15 mg, 0.08 mmol). The reaction mixture was stirred at 100° C. overnight. After cooling to room temperature, the mixture was purified by prep-HPLC to give the target product II-1 (3.5 mg, 5%).

MS m/z (ESI): 414.1 [M+H⁺]

¹H NMR (400 MHz, DMSO-d₆) δ 10.88 (s, 1H), 7.41-7.26 (m, 3H), 7.20 (dd, J=7.5, 1.6 Hz, 1H), 6.76 (dd, J=7.4, 1.6 Hz, 1H), 6.15 (t, J=7.0 Hz, 1H), 4.29 (dd, J=12.0, 5.0 Hz, 1H), 3.82 (d, J=11.5 Hz, 2H), 3.43 (s, 3H), 3.25-3.08 (m, 3H), 2.83-2.71 (m, 1H), 2.57-2.54 (m, 1H), 2.35-2.25 (m, 1H), 2.06-1.94 (m, 3H), 1.85-1.76 (m, 2H).

### Example 3

### 3-(2-Chloro-3-(1-(1,4-dimethyl-2-oxo-1,2-dihydropyridin-3-yl) piperidin-4-yl)phenyl) piperidine-2,6-dione (Compound II-2)

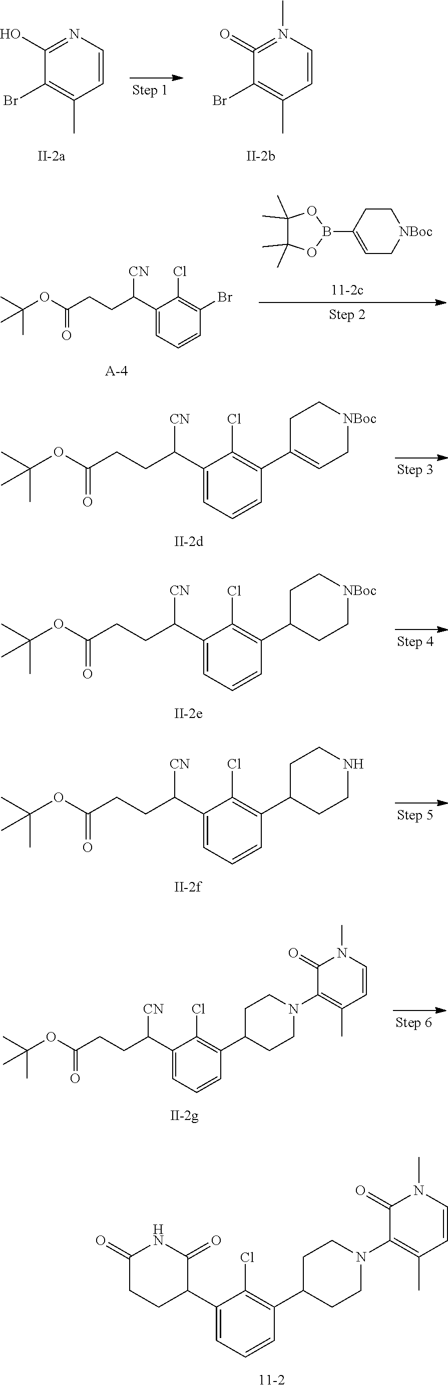

### Step 1

### 3-Bromo-1,4-dimethylpyridin-2 (1H)-one (II-2b)

To a solution of 3-bromo-4-methylpyridin-2-ol II-2a (3 g, 16 mmol) in THF (75 mL) at 0° C. was added sodium hydride (60%, 461 mg, 19 mmol), and the mixture was stirred at 0° C. for 30 minutes. The mixture was added with iodomethane (3.4 g, 24 mmol) and stirred at room temperature for 1 hour. After quenching with saturated ammonium chloride solution, the mixture was extracted with ethyl acetate. The organic phase was dried over anhydrous sodium sulfate and concentrated under reduced pressure. The residue was purified by silica gel column chromatography (petroleum ether/ethyl acetate=55/45 v/v) to give the target product II-2b (1.3 g, 40%).

MS m/z (ESI): 201.9 [M+H⁺]

### Step 2

### tert-Butyl 4-(3-(4-(tert-butoxy)-1-cyano-4-oxobutyl)-2-chlorophenyl)-3,6-dihydropyridine-1 (2H)-carboxylate (II-2d)

To a mixture of A-4 (1.6 g, 4.5 mmol), tert-butyl 4-(4,4,5,5-tetramethyl-1,3,2-dioxaborolan-2-yl)-3,6-dihydropyridine-1 (2H)-carboxylate II-2c (2.1 g, 6.7 mmol), potassium phosphate (2.8 g, 13.4 mmol), Pd(dppf)Cl₂ (326 mg, 0.45 mmol) and 1,4-dioxane (20 mL) was added water (2 mL). The mixture was stirred at 90° C. for 5 hours and concentrated under reduced pressure. The residue was purified by silica gel column chromatography (petroleum ether/ethyl acetate=5/1 v/v) to give the target product II-2d (2 g, 97%).

MS m/z (ESI): 361.1 [M+H⁺−100]

### Step 3

### tert-Butyl 4-(3-(4-(tert-butoxy)-1-cyano-4-oxobutyl)-2-chlorophenyl) piperidine-1-carboxylate (II-2e)

To a solution of II-2d (1.8 g, 3.9 mmol) in methanol (20 mL) was added platinum (IV) oxide (360 mg), and the mixture was stirred at room temperature under a hydrogen atmosphere for 3 hours. The mixture was filtered, and the filter cake was washed with methanol. The filtrate was concentrated under reduced pressure and the residue was purified by silica gel column chromatography (petroleum ether/ethyl acetate=9/1 v/v) to give the target product II-2e (1 g, 55%).

MS m/z (ESI): 363.1 [M+H⁺−100]

### Step 4

### tert-Butyl 4-(2-chloro-3-(piperidin-4-yl)phenyl)-4-cyanobutanoate (II-2f)

To a solution of II-2e (1 g, 2.2 mmol) in dichloromethane (10 mL) was added trifluoroacetic acid (2 mL), and the mixture was stirred at room temperature for 4 hours. After quenching with saturated aqueous sodium bicarbonate solution, the mixture was extracted with dichloromethane. The organic phase was dried over anhydrous sodium sulfate, filtered and concentrated under reduced pressure to give the target product II-2f (500 mg, 50%).

MS m/z (ESI): 363.2 [M+H⁺]

### Step 5

### tert-Butyl 4-(2-chloro-3-(1-(1,4-dimethyl-2-oxo-1,2-dihydropyridin-3-yl) piperidin-4-yl)phenyl)-4-cyanobutanoate (II-2g)

To a solution of II-2f (500 mg, 1.4 mmol), II-2b (278 mg, 1.4 mmol), Pd₂(dba)₃ (126 mg, 0.14 mmol) and Xantphos (80 mg, 0.14 mmol) in 1,4-dioxane (10 mL) was added cesium carbonate (1.4 g, 4.1 mmol), and the mixture was stirred at 100° C. for 5 hours. The mixture was concentrated under reduced pressure and the residue was purified by silica gel column chromatography (petroleum ether/ethyl acetate=3/2 v/v) to give the target product II-2g (100 mg, 15%).

MS m/z (ESI): 484.2 [M+H⁺]

### Step 6

### 3-(2-Chloro-3-(1-(1,4-dimethyl-2-oxo-1,2-dihydropyridin-3-yl) piperidin-4-yl)phenyl) piperidine-2,6-dione (II-2)

To a solution of II-2g (100 mg, 0.21 mmol) in acetic acid (2 mL) was added concentrated sulfuric acid (0.2 mL), and the mixture was stirred at 90° C. for 4 hours. The mixture was concentrated under reduced pressure and the residue was purified by prep-HPLC to give the target product II-2 (25 mg, 28%).

MS m/z (ESI): 428.2 [M+H⁺]

¹H NMR (400 MHz, DMSO-d₆) δ 10.88 (s, 1H), 7.40 (d, J=6.9 Hz, 1H), 7.37 (dd, J=7.8, 1.6 Hz, 1H), 7.32 (t, J=7.6 Hz, 1H), 7.19 (dd, J=7.5, 1.6 Hz, 1H), 6.05 (d, J=6.9 Hz, 1H), 4.29 (dd, J=12.0, 5.0 Hz, 1H), 3.59-3.48 (m, 2H), 3.37 (s, 3H), 3.13-3.02 (m, 1H), 2.83-2.69 (m, 3H), 2.59-2.53 (m, 1H), 2.36-2.23 (m, 1H), 2.19 (s, 3H), 2.05-1.96 (m, 1H), 1.83-1.65 (m, 4H).

### Example 4

### 3-(2-Chloro-3-(4-(1,4-dimethyl-2-oxo-1,2-dihydropyridin-3-yl) piperazin-1-yl)phenyl) piperidine-2,6-dione (Compound II-3)

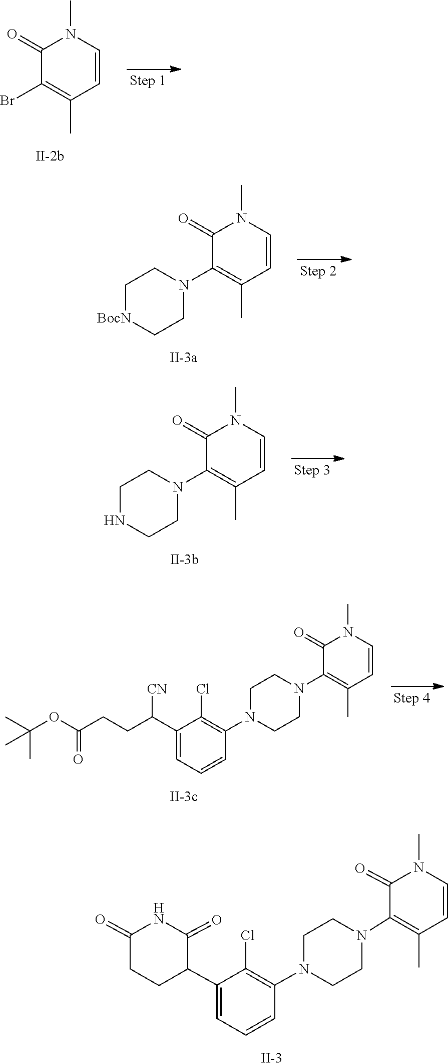

### Step 1

### tert-Butyl 4-(1,4-dimethyl-2-oxo-1,2-dihydropyridin-3-yl) piperazine-1-carboxylate (II-3a)

To a solution of II-2b (300 mg, 1.4 mmol), tert-butyl piperazine-1-carboxylate (359 mg, 1.3 mmol), Pd₂(dba)₃ (135 mg, 0.15 mmol) and Xantphos (86 mg, 0.15 mmol) in 1,4-dioxane (10 mL) was added cesium carbonate (1.5 g, 4.4 mmol), and the mixture was stirred at 110° C. for 6 hours. The mixture was concentrated under reduced pressure and the residue was purified by silica gel column chromatography (petroleum ether/ethyl acetate=1/1 v/v) to give the target product II-3a (300 mg, 66%).

MS m/z (ESI): 308.3 [M+H⁺]

### Step 2

### 1,4-Dimethyl-3-(piperazin-1-yl)pyridin-2 (1H)-one (II-3b)

A mixture of II-3a (300 mg, 0.98 mmol) and a solution of HCl in 1,4-dioxane (4 M, 10 mL) was stirred at room temperature for 2 hours. The mixture was concentrated under reduced pressure to give the target product II-3b as a hydrochloride salt (230 mg, 97%).

MS m/z (ESI): 208.2 [M+H⁺]

### Steps 3 to 4

### 3-(2-Chloro-3-(4-(1,4-dimethyl-2-oxo-1,2-dihydropyridin-3-yl) piperazin-1-yl)phenyl) piperidine-2,6-dione (II-3)

Compound II-3 was synthesized according to the procedures of Steps 5-6 for Example 3, but II-3b was used instead of II-2f and A-4 was used instead of II-2b.

MS m/z (ESI): 429.1 [M+H⁺]

¹H NMR (400 MHz, DMSO-d₆) δ 10.87 (s, 1H), 7.43 (d, J=6.9 Hz, 1H), 7.28 (t, J=7.8 Hz, 1H), 7.15 (d, J=8.0 Hz, 1H), 7.03-6.98 (m, 1H), 6.07 (d, J=6.9 Hz, 1H), 4.27 (dd, J=12.0, 5.1 Hz, 1H), 3.38 (s, 3H), 3.27-2.94 (m, 8H), 2.82-2.71 (m, 1H), 2.58-2.53 (m, 1H), 2.35-2.23 (m, 1H), 2.20 (s, 3H), 2.04-1.96 (m, 1H).

### Example 5

### 3-(2-Chloro-3-(4-(1-methyl-2-oxo-1,2-dihydropyridin-3-yl)cyclohexyl)phenyl) piperidine-2,6-dione (Compound II-4)

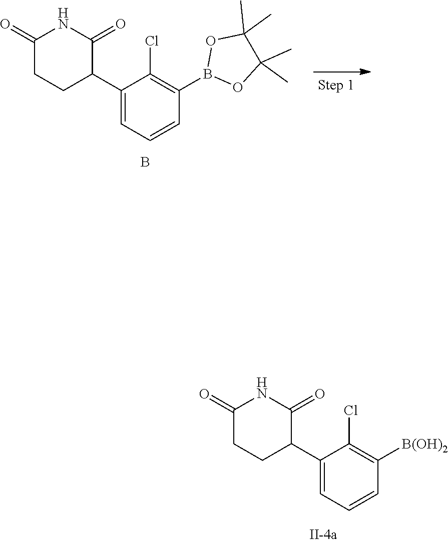

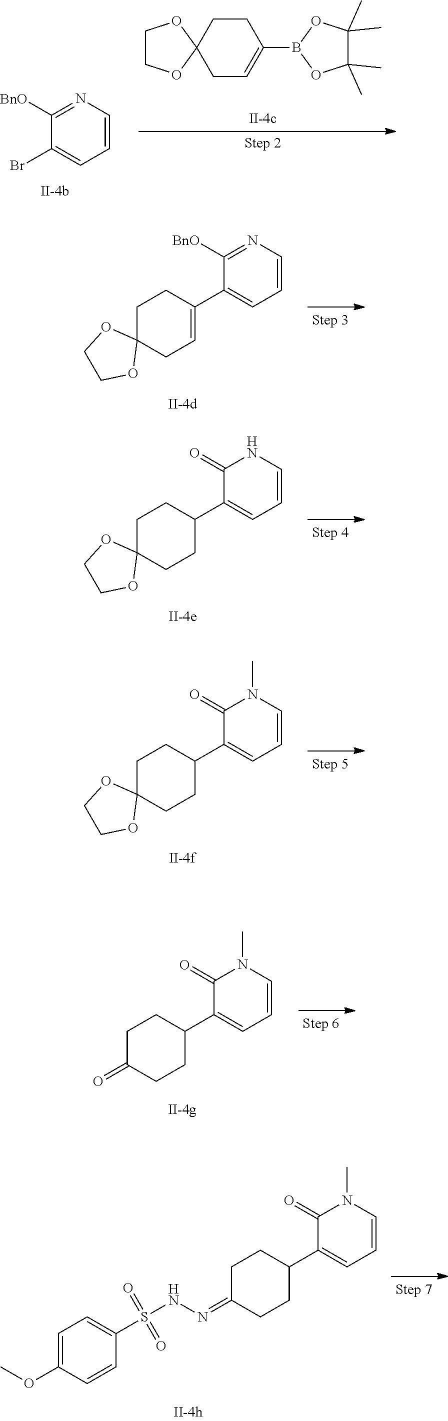

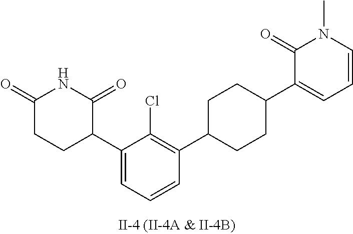 [SMILES: Cn1cccc(C2CCC(c3cccc(C4CCC(=O)NC4=O)c3Cl)CC2)c1=O]

### Step 1

### (2-Chloro-3-(2,6-dioxopiperidin-3-yl)phenyl) boronic acid (II-4a)

To a solution of B (2 g, 5.2 mmol) in THF (25 mL) and water (5 mL) was added sodium periodate (2.8 g, 13.0 mmol), and the mixture was stirred at room temperature overnight. After quenching with dilute hydrochloric acid solution (1 N), the mixture was concentrated under reduced pressure and the residue was purified by prep-HPLC to give the target product II-4a (400 mg, 29%).

### Step 2

### 2-(Benzyloxy)-3-(1,4-dioxaspiro[4.5]dec-7-en-8-yl)pyridine (II-4d)

To a mixture of 2-(benzyloxy)-3-bromopyridine II-4b (1.0 g, 3.8 mmol), 4 4,4,5,5-tetramethyl-2-(1,4-dioxaspiro[4.5]dec-7-en-8-yl)-1,3,2-dioxaborolane II-4c (1.5 g, 5.7 mmol), potassium phosphate (2.4 g, 11 mmol), 1,4-dioxane (20 mL) and water (2 mL) was added Pd(dppf)Cl₂ (0.30 g, 0.38 mmol). The mixture was stirred at 100° C. for 16 hours, diluted with water and extracted with ethyl acetate. The organic phase was concentrated under reduced pressure and the residue was purified by silica gel column chromatography (petroleum ether/ethyl acetate=100/0 to 30/70 v/v) to give the target product II-4d (1.4 g, 100%).

MS m/z (ESI): 324.1 [M+H⁺]

### Step 3

### 3-(1,4-Dioxaspiro[4.5]decan-8-yl)pyridin-2 (1H)-one (II-4e)

To a solution of II-4d (1.4 g, 4.3 mmol) in methanol (20 mL) was added palladium on carbon (10%, containing water 55%, 0.70 g), and the mixture was stirred at room temperature under a hydrogen atmosphere for 2 hours. The mixture was filtered, and the filter cake was washed with methanol. The filtrate was concentrated under reduced pressure to give the target product II-4e (crude, 900 mg). The product was used directly in the next step without further purification.

MS m/z (ESI): 236.1 [M+H⁺]

### Step 4

### 1-Methyl-3-(1,4-dioxaspiro[4.5]decan-8-yl)pyridin-2 (1H)-one (II-4f)

To a mixture of II-4e (0.85 g, 3.6 mmol), potassium carbonate (1.5 g, 11 mmol) and acetonitrile (20 mL) was added iodomethane (0.70 mL, 11 mmol). The mixture was stirred in a sealed tube at 80° C. for 16 hours. After cooling to room temperature, the mixture was concentrated under reduced pressure, added with water and extracted with dichloromethane. The organic phase was concentrated under reduced pressure and the residue was purified by silica gel column chromatography (methanol/dichloromethane=0/100 to 15/85 v/v) to give the target product II-4f (540 mg, 60%).

MS m/z (ESI): 250.1 [M+H⁺]

### Step 5

### 1-Methyl-3-(4-oxocyclohexyl)pyridin-2 (1H)-one (II-4g)

To a solution of II-4f (0.54 g, 2.2 mmol) in acetonitrile (4 mL) was added diluted hydrochloric acid solution (1 N, 4 mL), and the mixture was stirred at room temperature for 1 hour. After quenching with saturated aqueous sodium bicarbonate solution, the mixture was extracted with ethyl acetate. The organic phase was concentrated under reduced pressure and the residue was purified by silica gel column chromatography (methanol/dichloromethane=0/100 to 15/85 v/v) to give the target product II-4g (390 mg, 88%).

MS m/z (ESI): 206.1 [M+H⁺]

### Step 6

### 4-Methoxy-N′-(4-(1-methyl-2-oxo-1,2-dihydropyridin-3-yl)cyclohexylidene)benzenesulfonohydrazide (II-4h)

To a solution of II-4g (0.39 g, 1.9 mmol) in toluene (5 mL) was added 4-methoxybenzenesulfonohydrazide (0.40 g, 2.0 mmol), and the mixture was stirred at 50° C. for 16 hours. The mixture was concentrated under reduced pressure, added with MTBE and filtered. The filter cake was washed with MTBE and dried to give the target product II-4h (610 mg, 82%).

MS m/z (ESI): 390.1 [M+H⁺]

### Step 7

### 3-(2-Chloro-3-(4-(1-methyl-2-oxo-1,2-dihydropyridin-3-yl)cyclohexyl)phenyl) piperidine-2,6-dione (II-4)

To a mixture of II-4h (100 mg, 0.25 mmol), II-4a (82 mg, 0.31 mmol) and 1,4-dioxane (5 mL) was added cesium carbonate (250 mg, 0.77 mmol), and the mixture was stirred at 110° C. for 16 hours. The mixture was diluted with water and extracted with ethyl acetate. The organic phase was concentrated under reduced pressure and the residue was purified by prep-HPLC to give the target product II-4, which consisted of isomers II-4A (13 mg, 13%) and II-4B (5 mg, 5%).

Characterization data for II-4A:

MS m/z (ESI): 413.1 [M+H⁺]

¹H NMR (400 MHz, DMSO-d₆) δ 7.55 (dd, J=6.7, 1.7 Hz, 1H), 7.46-7.44 (m, 1H), 7.35-7.27 (m, 3H), 6.19 (t, J=6.8 Hz, 1H), 4.25 (dd, J=12.3, 5.2 Hz, 1H), 3.41 (s, 3H), 3.11-3.04 (m, 1H), 2.93-2.78 (m, 1H), 2.68-2.60 (m, 1H), 2.40-2.19 (m, 3H), 2.13-1.89 (m, 4H), 1.77-1.63 (m, 2H), 1.43-1.33 (m, 2H).

Characterization data for II-4B:

MS m/z (ESI): 413.1 [M+H⁺]

¹H NMR (400 MHz, DMSO-d₆) δ 7.53 (dd, J=6.7, 1.9 Hz, 1H), 7.37-7.28 (m, 3H), 7.24 (dd, J=6.9, 1.7 Hz, 1H), 6.15 (t, J=6.8 Hz, 1H), 4.28 (dd, J=12.2, 5.2 Hz, 1H), 3.42 (s, 3H), 2.91-2.83 (m, 1H), 2.71-2.62 (m, 2H), 2.40-2.24 (m, 4H), 2.01-1.94 (m, 3H), 1.87-1.78 (m, 2H), 1.66-1.58 (m, 2H).

### Example 6

### 3-(2-Chloro-3-(4-((5-cyclopropylpyridin-2-yl)methyl)-3-oxopiperazin-1-yl)phenyl) piperidine-2,6-dione (Compound II-5)

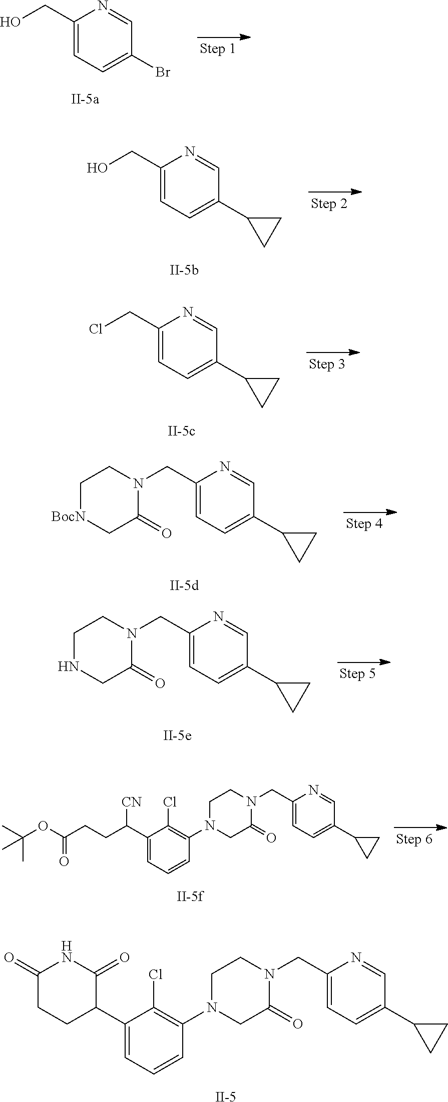

### Step 1

### (5-Cyclopropylpyridin-2-yl) methanol (II-5b)

A mixture of II-5a (2.0 g, 10.6 mmol), cyclopropylboronic acid (1.8 g, 21.3 mmol), potassium phosphate (6.7 g, 31.8 mmol), Pd(dppf)Cl₂ (774 mg, 1.1 mmol), 1,4-dioxane (20 mL) and water (6 mL) was stirred at 95° C. overnight. The mixture was added with water and extracted with ethyl acetate. The organic phase was concentrated under reduced pressure, and the residue was purified by silica gel column chromatography (petroleum ether/ethyl acetate=15/85 v/v) to give the target product II-5b (549 mg, 35%).

MS m/z (ESI): 150.1 [M+H⁺]

### Step 2

### 2-(Chloromethyl)-5-cyclopropylpyridine (II-5c)

A mixture of II-5b (549 mg, 3.7 mmol) and thionyl chloride (5 mL) was stirred at room temperature for 1 hour. The mixture was concentrated under reduced pressure to give the target product II-5c (610 mg, crude). The product was used directly in the next step without further purification.

MS m/z (ESI): 168.1 [M+H⁺]

### Step 3

### tert-Butyl 4-((5-cyclopropylpyridin-2-yl)methyl)-3-oxopiperazine-1-carboxylate (II-5d)

To a solution of tert-butyl 3-oxopiperazine-1-carboxylate (1.0 g, 5.0 mmol) in DMF (25 mL) at 0° C. was added sodium hydride (60%, 400 mg, 10 mmol), and the reaction mixture was stirred at room temperature for 1 hour. II-5c (919 mg, 5.5 mmol) was added and stirring was continued at room temperature for 4 hours. After quenching with saturated aqueous ammonium chloride solution, the mixture was extracted with ethyl acetate. The organic phase was concentrated under reduced pressure and the residue was purified by silica gel column chromatography (petroleum ether/ethyl acetate=100/0 to 0/100 v/v) to give the target product II-5d (744 mg, 45%).

MS m/z (ESI): 332.2 [M+H⁺]

### Steps 4 to 6

### 3-(2-Chloro-3-(4-((5-cyclopropylpyridin-2-yl)methyl)-3-oxopiperazin-1-yl)phenyl) piperidine-2,6-dione (II-5)

Compound II-5 was synthesized according to the procedures of Steps 4-6 for Example 3, but II-5d was used instead of II-2e and A-4 was used instead of II-2b.

MS m/z (ESI): 453.2 [M+H⁺]

¹H NMR (400 MHz, DMSO-d₆) δ 10.88 (s, 1H), 8.36 (d, J=2.0 Hz, 1H), 7.41 (dd, J=8.1, 2.3 Hz, 1H), 7.31 (t, J=7.8 Hz, 1H), 7.20-7.13 (m, 2H), 7.07 (d, J=7.7 Hz, 1H), 4.61 (s, 2H), 4.27 (dd, J=12.1, 5.0 Hz, 1H), 3.71 (s, 2H), 3.44 (t, J=5.2 Hz, 2H), 3.32-2.26 (m, 2H), 2.82-2.72 (m, 1H), 2.58-2.53 (m, 1H), 2.36-2.22 (m, 1H), 2.05-1.90 (m, 2H), 1.04-0.95 (m, 2H), 0.76-0.69 (m, 2H).

### Example 7

### 3-(2-Chloro-3-(2-oxo-1-phenylpiperidin-4-yl)phenyl) piperidine-2,6-dione (Compound II-6)

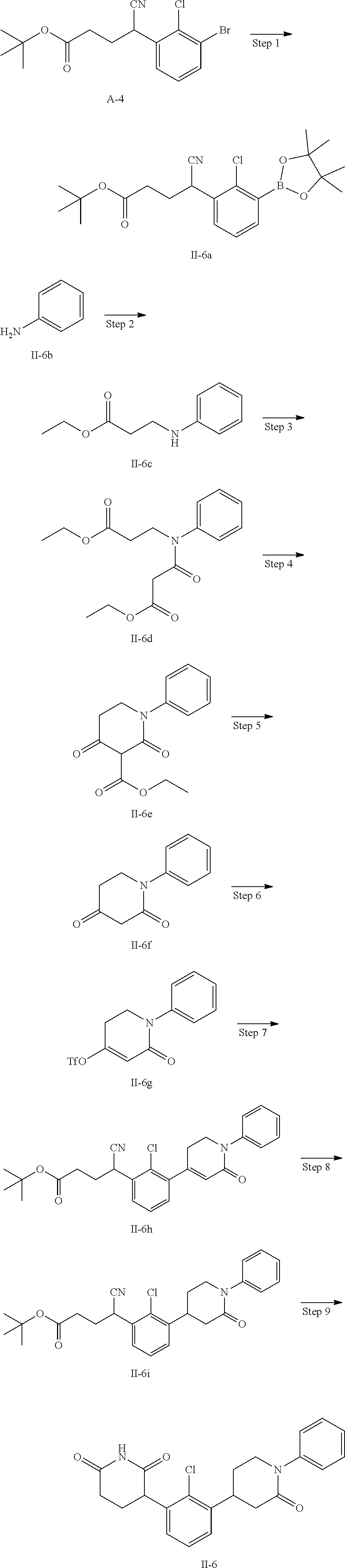

### Step 1

### tert-Butyl 4-(2-chloro-3-(4,4,5,5-tetramethyl-1,3,2-dioxaborolan-2-yl)phenyl)-4-cyanobutanoate (II-6a)

To a solution of A-4 (2.0 g, 5.6 mmol) in 1,4-dioxane (20 mL) were added with 4,4,4′,4′,5,5,5′,5′-octamethyl-2,2′-bi (1,3,2-dioxaborolane) (1.7 g, 6.7 mmol), Pd(dppf)Cl₂ (409 mg, 0.56 mmol) and potassium acetate (1.6 g, 16.8 mmol), and the reaction mixture was stirred at 100° C. overnight. The mixture was concentrated under reduced pressure, and the residue was purified by silica gel column chromatography (petroleum ether/ethyl acetate=54/46 v/v) to give the target product II-6a (1.6 g, 73%).

MS m/z (ESI): 406.1 [M+H⁺]

### Step 2

### Ethyl 3-(phenylamino) propanoate (II-6c)

To a solution of II-6b (8.0 g, 85.9 mmol) in ethanol (40 mL) were added with ethyl acrylate (12.9 g, 129 mmol) and hydrochloric acid (12 N, 40 mL), and the reaction mixture was stirred at 80° C. overnight. The mixture was concentrated under reduced pressure, added with water, and extracted with ethyl acetate. The organic phase was concentrated under reduced pressure, and the residue was purified by silica gel column chromatography (petroleum ether) to give the target product II-6c (7.0 g, 42%).

MS m/z (ESI): 194.1 [M+H⁺]

### Step 3

### Ethyl 3-((3-ethoxy-3-oxopropyl) (phenyl)amino)-3-oxopropanoate (II-6d)

To a solution of II-6c (7.0 g, 36.1 mmol) and triethylamine (5.5 g, 54.1 mmol) in dichloromethane (70 mL) at 0° C. was added ethyl 3-chloro-3-oxopropanoate (6.0 g, 39.2 mmol), and the reaction mixture was stirred at 0° C. for 2 hours. Water was added and the resulting mixture was extracted with ethyl acetate. The organic phase was concentrated under reduced pressure, and the residue was purified by silica gel column chromatography (petroleum ether/ethyl acetate=90/10) to give the target product II-6d (7.6 g, 69%).

MS m/z (ESI): 308.1 [M+H⁺]

### Step 4

### Ethyl 2,4-dioxo-1-phenylpiperidine-3-carboxylate (II-6e)

A mixture of II-6d (7.6 g, 24.8 mmol), a solution of sodium ethoxide in ethanol (20%, 16.8 g, 49.5 mmol) and ethanol (76 mL) was stirred at 80° C. overnight. After cooling to room temperature, the mixture was added with water and extracted with ethyl acetate. The organic phase was concentrated under reduced pressure, and the residue was purified by silica gel column chromatography (petroleum ether/ethyl acetate=90/10) to give the target product II-6e (1.2 g, 18%).

MS m/z (ESI): 262.1 [M+H⁺]

### Step 5

### 1-Phenylpiperidine-2,4-dione (II-6f)

A mixture of II-6e (1.2 g, 4.5 mmol), acetonitrile (12 mL) and water (1 mL) was stirred at 80° C. for 2 hours. The mixture was concentrated under reduced pressure to yield the target product II-6f (720 mg, 84%). The product was used directly in the next step without further purification.

MS m/z (ESI): 190.3 [M+H⁺]

### Step 6

### 6-Oxo-1-phenyl-1,2,3,6-tetrahydropyridin-4-yl trifluoromethanesulfonate (II-6g)

A mixture of II-6f (720 mg, 3.8 mmol), PhNTf₂ (1.4 g, 3.9 mmol), triethylamine (1.2 g, 11.4 mmol) and THF (7.2 mL) was stirred at room temperature for 2 hours. The mixture was added with water and extracted with ethyl acetate. The organic phase was concentrated under reduced pressure, and the residue was purified by silica gel column chromatography (petroleum ether/ethyl acetate=92/8) to give the desired product II-6g (611 mg, 50%).

MS m/z (ESI): 322.0 [M+H⁺]

### Step 7

### tert-Butyl 4-(2-chloro-3-(6-oxo-1-phenyl-1,2,3,6-tetrahydropyridin-4-yl)phenyl)-4-cyanobutanoate (II-6h)

A mixture of II-6g (611 mg, 1.9 mmol), II-6a (848 mg, 2.1 mmol), potassium phosphate (1.2 g, 5.7 mmol), Pd(dppf)Cl₂ (139 mg, 0.19 mmol), 1,4-dioxane (18 mL) and water (6 mL) was stirred at 100° C. overnight. The mixture was cooled to room temperature, added with water and extracted with ethyl acetate. The organic phase was concentrated under reduced pressure, and the residue was purified by silica gel column chromatography (petroleum ether/ethyl acetate=80/20) to yield the target product II-6h (430 mg, 50%).

MS m/z (ESI): 451.2 [M+H⁺]

### Step 8

### tert-Butyl 4-(2-chloro-3-(2-oxo-1-phenylpiperidin-4-yl)phenyl)-4-cyanobutanoate (II-6i)

A mixture of II-6h (430 mg, 0.95 mmol), platinum dioxide (129 mg) and methanol (4 mL) was stirred at room temperature under a hydrogen atmosphere for 1 hour. After filtration, the filtrate was concentrated under reduced pressure to give the target product II-6i (410 mg, 95%).

MS m/z (ESI): 397.1 [M+H⁺-56]

### Step 9

### 3-(2-Chloro-3-(2-oxo-1-phenylpiperidin-4-yl)phenyl) piperidine-2,6-dione (II-6)

A mixture of II-6i (385 mg, 0.84 mmol), concentrated sulfuric acid (400 μL) and acetic acid (4 mL) was stirred at 90° C. for 3 hours. The mixture was purified by prep-HPLC to give the target product II-6 as two isomers, II-6A (46.2 mg, 14%) and II-6B (34.7 mg, 10%).

Characterization data for II-6A:

MS m/z (ESI): 397.2 [M+H⁺]

¹H NMR (400 MHz, DMSO-d₆) δ 10.90 (s, 1H), 7.43-7.25 (m, 8H), 4.32 (dd, J=12.1, 5.0 Hz, 1H), 3.91-3.83 (m, 1H), 3.76-3.67 (m, 1H), 3.62-3.57 (m, 1H), 2.85-2.73 (m, 1H), 2.72-2.64 (m, 1H), 2.62-2.53 (m, 2H), 2.38-2.09 (m, 3H), 2.07-1.97 (m, 1H).

Characterization data for II-6B:

MS m/z (ESI): 397.2 [M+H⁺]

¹H NMR (400 MHz, DMSO-d₆) δ 10.90 (s, 1H), 7.49-7.21 (m, 8H), 4.31 (dd, J=12.1, 5.0 Hz, 1H), 3.91-3.80 (m, 1H), 3.77-3.66 (m, 1H), 3.64-3.55 (m, 1H), 2.85-2.66 (m, 2H), 2.63-2.54 (m, 2H), 2.37-2.08 (m, 3H), 2.07-1.96 (m, 1H).

### Example 8

### 3-(2-Chloro-3-(1-((5-cyclopropylpyridin-2-yl)methyl) piperidin-4-yl)phenyl) piperidine-2,6-dione (Compound II-7)

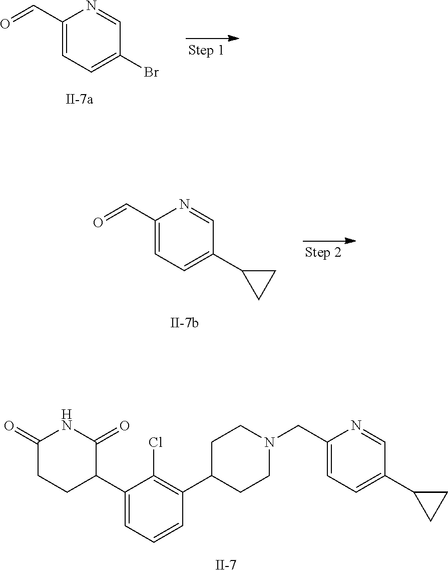

### Step 1

### 5-Cyclopropylpicolinaldehyde (II-7b)

A mixture of II-7a (1 g, 5.4 mmol), cyclopropylboronic acid (923 mg, 10.8 mmol), potassium phosphate (3.4 g, 16.1 mmol), Pd(dppf)Cl₂ (394 mg, 0.54 mmol), 1,4-dioxane (10 mL) and water (1 mL) was stirred at 90° C. for 6 hours. The mixture was concentrated under reduced pressure. The residue was purified by silica gel column chromatography (petroleum ether/ethyl acetate=13/1 v/v) to give the target product II-7b (130 mg, 16%).

MS m/z (ESI): 148.2 [M+H⁺]

### Step 2

### 3-(2-Chloro-3-(1-((5-cyclopropylpyridin-2-yl)methyl) piperidin-4-yl)phenyl) piperidine-2,6-dione (II-7)

To a mixture of II-7b (68 mg, 0.46 mmol), II-1d (80 mg, 0.23 mmol), DMF (2 mL) and dichloromethane (4 mL) was added sodium triacetoxyhydroborate (244 mg, 1.2 mmol). The reaction mixture was stirred at room temperature for 3 hours and concentrated under reduced pressure. The residue was purified by prep-HPLC to give the target product II-7 (28 mg, 27%).

MS m/z (ESI): 438.2 [M+H⁺]

¹H NMR (400 MHz, DMSO-d₆) δ 10.87 (s, 1H), 8.32 (d, J=2.0 Hz, 1H), 7.41-7.26 (m, 4H), 7.17 (dd, J=7.4, 1.8 Hz, 1H), 4.27 (dd, J=12.0, 5.0 Hz, 1H), 3.58 (s, 2H), 3.02-2.89 (m, 3H), 2.82-2.70 (m, 1H), 2.58-2.53 (m, 1H), 2.35-2.20 (m, 1H), 2.14 (t, J=11.2 Hz, 2H), 2.04-1.88 (m, 2H), 1.79-1.61 (m, 4H), 1.03-0.94 (m, 2H), 0.74-0.68 (m, 2H).

### Example 9

### 3-(2-Chloro-3-(4-(2-oxopyridin-1 (2H)-yl) piperidin-1-yl)phenyl) piperidine-2,6-dione (Compound II-8)

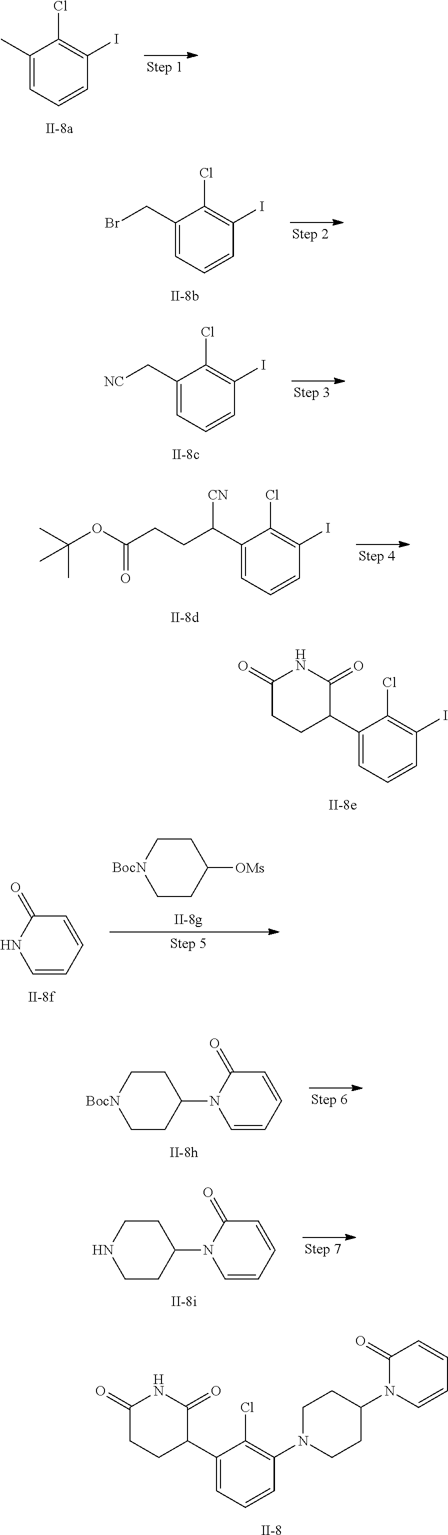

### Steps 1 to 4

### 3-(2-Chloro-3-iodophenyl) piperidine-2,6-dione (II-8e)

II-8e was synthesized according to the procedures of Steps 1˜4 for A, but II-8a was used instead of A-1.

MS m/z (ESI): 350.0 [M+H⁺]

### Step 5

### tert-Butyl 4-(2-oxopyridin-1 (2H)-yl) piperidine-1-carboxylate (II-8h)

To a mixture of pyridin-2 (1H)-one II-8f (1.5 g, 16 mmol), tert-butyl 4-((methylsulfonyl)oxy) piperidine-1-carboxylate II-8g (4.4 g, 16 mmol) and DMF (30 mL) was added potassium carbonate (6.5 g, 48 mmol). The reaction mixture was stirred at 100° C. for 12 hours, added with water and extracted with ethyl acetate. The organic phase was concentrated under reduced pressure and the residue was purified by silica gel column chromatography (petroleum ether/ethyl acetate=1/1 v/v) to afford the target product II-8h (780 mg, 18%).

MS m/z (ESI): 223.1 [M+H⁺-56]

### Step 6

### 1-(Piperidin-4-yl)pyridin-2 (1H)-one (II-8i)

A mixture of II-8h (780 mg, 2.8 mmol) and a solution of HCl in 1,4-dioxane (4 M, 10 mL) was stirred at room temperature for 3 hours. The mixture was concentrated under reduced pressure to give the target product II-8i as a hydrochloride salt (600 mg, 100%).

MS m/z (ESI): 179.2 [M+H⁺]

### Step 7

### 3-(2-Chloro-3-(4-(2-oxopyridin-1 (2H)-yl) piperidin-1-yl)phenyl) piperidine-2,6-dione (II-8)

To a mixture of II-8e (150 mg, 0.43 mmol), II-8i (92 mg, 0.43 mmol), copper (I) iodide (8 mg, 0.043 mmol) and DMSO (5 mL) were added L-proline (5 mg, 0.043 mmol) and potassium carbonate (178 mg, 1.3 mmol). The reaction mixture was stirred at 100° C. for 12 hours, added with water and extracted with ethyl acetate. The organic phase was concentrated under reduced pressure and the residue was purified by prep-HPLC to afford the target product II-8 (3 mg, 2%).

MS m/z (ESI): 400.1 [M+H⁺]

¹H NMR (400 MHz, DMSO-d₆) δ 10.87 (s, 1H), 7.80 (d, J=6.9 Hz, 1H), 7.44-7.38 (m, 1H), 7.29 (t, J=7.8 Hz, 1H), 7.18 (d, J=6.7 Hz, 1H), 7.02 (d, J=6.4 Hz, 1H), 6.41 (d, J=9.1 Hz, 1H), 6.30-6.23 (m, 1H), 4.92-4.79 (m, 1H), 4.32-4.25 (m, 1H), 2.91-2.82 (m, 2H), 2.81-2.72 (m, 1H), 2.57-2.52 (m, 1H), 2.35-2.23 (m, 1H), 2.07-1.96 (m, 3H), 1.89-1.82 (m, 2H).

### Example 10

### 3-(2-Chloro-3-(1-(5-cyclopropylpicolinoyl) piperidin-4-yl)phenyl) piperidine-2,6-dione

### (Compound II-9)

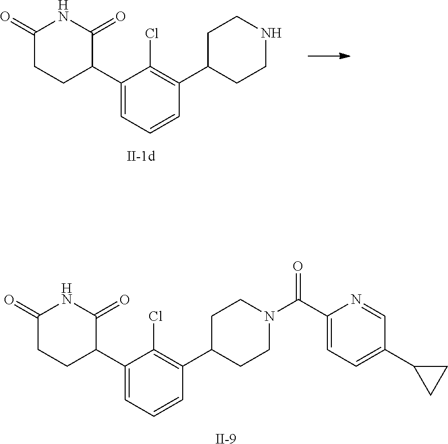

To a mixture of II-1d (hydrochloride salt, 30 mg, 0.087 mmol), 5-cyclopropylpicolinic acid (hydrochloride salt, 21 mg, 0.1 mmol), DIPEA (34 mg, 0.26 mmol) and DMF (2 mL) was added HATU (49 mg, 0.13 mmol). The reaction mixture was stirred at room temperature for 1 hour and purified by prep-HPLC to afford the target product II-9 (18 mg, 46%).

MS m/z (ESI): 452.1 [M+H⁺]

¹H NMR (400 MHz, DMSO-d₆) δ 10.88 (s, 1H), 8.41 (d, J=1.7 Hz, 1H), 7.54 (dd, J=8.1, 2.2 Hz, 1H), 7.48 (d, J=8.1 Hz, 1H), 7.37-7.28 (m, 2H), 7.20 (dd, J=7.2, 1.9 Hz, 1H), 4.67 (d, J=12.0 Hz, 1H), 4.28 (dd, J=12.0, 5.0 Hz, 1H), 3.89 (d, J=12.6 Hz, 1H), 3.19 (t, J=12.0 Hz, 1H), 2.90 (t, J=12.5 Hz, 1H), 2.82-2.71 (m, 1H), 2.58-2.53 (m, 1H), 2.34-2.21 (m, 1H), 2.05-1.95 (m, 2H), 1.89 (t, J=10.4 Hz, 1H), 1.76-1.53 (m, 3H), 1.08-1.00 (m, 2H), 0.84-0.76 (m, 2H).

### Example 11

### 3-(2-Chloro-3-(1-(2-methoxypyridin-3-yl) piperidin-4-yl)phenyl) piperidine-2,6-dione (Compound II-10)

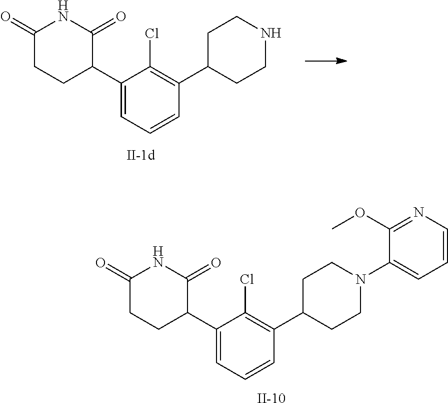

To a mixture of II-1d (40 mg, 0.12 mmol), 3-iodo-2-methoxypyridine (55 mg, 0.23 mmol), potassium carbonate (48 mg, 0.35 mmol), L-proline (2 mg, 0.012 mmol) and DMSO (3 mL) was added copper (I) iodide (2 mg, 0.012 mmol). The reaction mixture was stirred at 100° C. overnight, added with water and extracted with ethyl acetate. The organic phase was concentrated under reduced pressure and the residue was purified by prep-HPLC to afford the target product II-10 (15 mg, 31%).

MS m/z (ESI): 414.1 [M+H⁺]

¹H NMR (400 MHz, DMSO-d₆) δ 10.89 (s, 1H), 7.75 (dd, J=4.9, 1.5 Hz, 1H), 7.43-7.37 (m, 1H), 7.32 (t, J=7.7 Hz, 1H), 7.25 (dd, J=7.7, 1.4 Hz, 1H), 7.24-7.16 (m, 1H), 6.92 (dd, J=7.6, 4.9 Hz, 1H), 4.29 (dd, J=12.0, 5.0 Hz, 1H), 3.89 (s, 3H), 3.58 (d, J=11.3 Hz, 2H), 3.20-3.08 (m, 1H), 2.83-2.63 (m, 3H), 2.59-2.53 (m, 1H), 2.36-2.22 (m, 1H), 2.06-1.96 (m, 1H), 1.92-1.76 (m, 4H).

Compound II-11 was synthesized according to the procedures for Example 11 but a different reactant was used instead of 3-iodo-2-methoxypyridine.

| | | Compound replacing 3- | |
|---|---|---|---|
| Compound | | iodo-2- | MS m/z |
| Number | Compound Structure | methoxypyridine | (ESI) |
| II-11 | 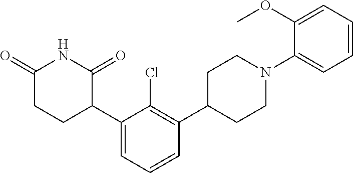 [SMILES: COc1ccccc1N1CCC(c2cccc(C3CCC(=O)NC3=O)c2Cl)CC1] | 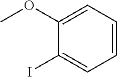 | 413.1 [M + H⁺] |

The NMR data for compound II-11 are as follows:

| Compound | ¹H NMR |
|---|---|
| 3-(2-chloro-3-(1-(2- | ¹H NMR (400 MHz, DMSO-d₆) δ 10.88 (s, 1H), 7.41 (dd, J = |
| methoxyphenyl)piperidin- | 7.7, 1.4 Hz, 1H), 7.32 (t, J = 7.7 Hz, 1H), 7.20 (dd, J = 7.6, |
| 4-yl)phenyl)piperidine- | 1.5 Hz, 1H), 6.98-6.84 (m, 4H), 4.29 (dd, J = 12.0, 5.0 Hz, |
| 2,6-dione (II-11) | 1H), 3.80 (s, 3H), 3.50 (d, J = 10.8 Hz, 2H), 3.18-3.07 (m, |
| | 1H), 2.84-2.63 (m, 3H), 2.59-2.53 (m, 1H), 2.36-2.23 |
| | (m, 1H), 2.05-1.96 (m, 1H), 1.89-1.78 (m, 4H). |

### Example 12

### 3-(2-Chloro-3-(4-(1,4-dimethyl-2-oxo-1,2-dihydropyridin-3-yl)-3-oxopiperazin-1-yl)phenyl) piperidine-2,6-dione (Compound II-12)

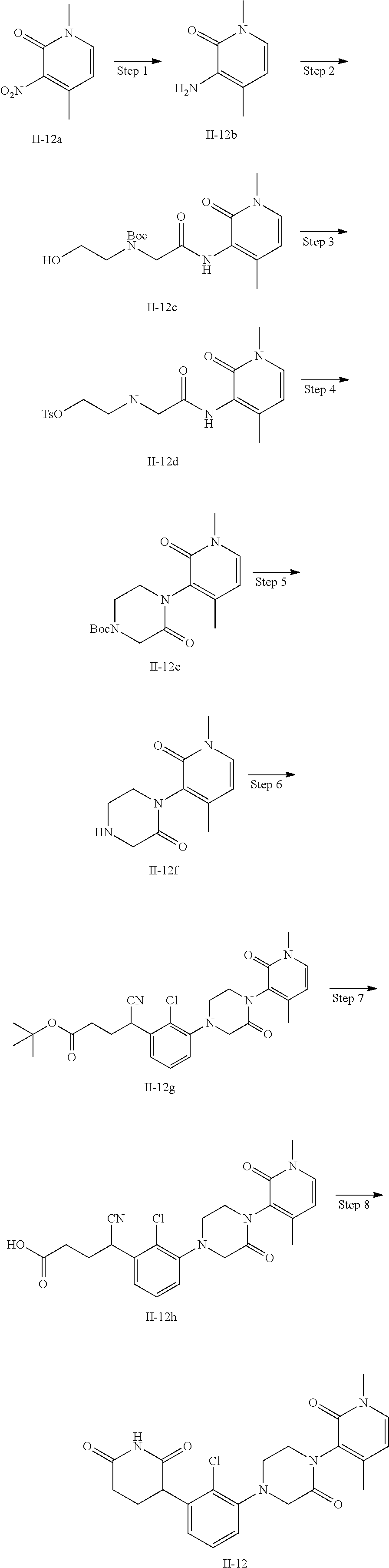

### Step 1

### 3-Amino-1,4-dimethylpyridin-2 (1H)-one (II-12b)

A mixture of II-12a (857 mg, 5.1 mmol), iron powder (2.85 g, 57.0 mmol), saturated aqueous ammonium chloride solution (5 mL) and ethanol (20 mL) was stirred at 80° C. for 3 hours. The reaction mixture was added with water and extracted with ethyl acetate. The organic phase was concentrated under reduced pressure and the residue was purified by silica gel column chromatography (petroleum ether/ethyl acetate=100/0 to 0/100 v/v) to afford the target product II-12b (587 mg, 83%).

MS m/z (ESI): 139.1 [M+H⁺]

### Step 2

### tert-Butyl(2-((1,4-dimethyl-2-oxo-1,2-dihydropyridin-3-yl)amino)-2-oxoethyl) (2-hydroxyethyl) carbamate (II-12c)

To a solution of II-12b (540 mg, 3.9 mmol) in THF (20 mL) at 0° C. was added a solution of isopropylmagnesium chloride in THF (2.0 M, 3.9 mL, 7.8 mmol). The reaction mixture was stirred at 0° C. for 30 minutes, added with tert-butyl 2-oxomorpholine-4-carboxylate (862 mg, 4.3 mmol) and stirred at room temperature overnight. After quenching with saturated aqueous ammonium chloride solution, the mixture was extracted with ethyl acetate. The organic phase was concentrated under reduced pressure and the residue was purified by silica gel column chromatography (petroleum ether/ethyl acetate=100/0 to 0/100 v/v) to afford the target product II-12c (1.1 g, 83%).

MS m/z (ESI): 340.3 [M+H⁺]

### Step 3

### 2-((tert-Butoxycarbonyl) (2-((1,4-dimethyl-2-oxo-1,2-dihydropyridin-3-yl)amino)-2-oxoethyl)amino)ethyl 4-methylbenzenesulfonate (II-12d)

A mixture of II-12c (210 mg, 0.62 mmol), DMAP (7.3 mg, 0.06 mmol), triethylamine (260 μL, 1.9 mmol), TsCl (177 mg, 0.93 mmol) and dichloromethane (3 mL) was stirred at room temperature for 2 hours. The reaction was quenched with saturated aqueous ammonium chloride solution and the mixture was extracted with dichloromethane. The organic phase was concentrated under reduced pressure and the residue was purified by silica gel column chromatography (dichloromethane/methanol=100/0 to 90/10 v/v) to afford the target product II-12d (270 mg, 88%).

MS m/z (ESI): 494.2 [M+H⁺]

### Step 4

### tert-Butyl 4-(1,4-dimethyl-2-oxo-1,2-dihydropyridin-3-yl)-3-oxopiperazine-1-carboxylate (II-12e)

To a solution of II-12d (0.27 g, 0.55 mmol) in THF (2.7 mL) at 0° C. was added sodium hydride (60%, 33 mg, 0.82 mmol). The mixture was stirred at room temperature for 30 minutes, quenched with saturated aqueous ammonium chloride solution and extracted with ethyl acetate. The organic phase was concentrated under reduced pressure and the residue was purified by silica gel column chromatography (dichloromethane/methanol=100/0 to 90/10 v/v) to afford the target product II-12e (78 mg, 44%).

MS m/z (ESI): 266.1 [M+H⁺-56]

### Step 5

### 1-(1,4-Dimethyl-2-oxo-1,2-dihydropyridin-3-yl) piperazin-2-one (II-12f)

A mixture of II-12e (78 mg, 0.24 mmol), trifluoroacetic acid (1 mL) and dichloromethane (1 mL) was stirred at room temperature for 1 hour. The mixture was concentrated under reduced pressure to give the target product II-12f (crude product, 85 mg). The product was used directly in the next step without further purification.

MS m/z (ESI): 222.3 [M+H⁺]

### Step 6

### tert-Butyl 4-(2-chloro-3-(4-(1,4-dimethyl-2-oxo-1,2-dihydropyridin-3-yl)-3-oxopiperazin-1-yl)phenyl)-4-cyanobutanoate (II-12g)

A mixture of II-12f (crude, 85 mg, 0.24 mmol), A-4 (129 mg, 0.36 mmol), cesium carbonate (429 mg, 1.3 mmol), Pd₂(dba)₃ (22 mg, 0.024 mmol), Xantphos (14 mg, 0.024 mmol) and 1,4-dioxane (3 mL) was stirred overnight at 100° C. The mixture was added with water and extracted with dichloromethane. The organic phase was concentrated under reduced pressure and the residue was purified by silica gel column chromatography (dichloromethane/methanol=94/6 v/v) to tive the target product II-12g (73 mg, 61% in two steps).

MS m/z (ESI): 499.3 [M+1]

### Step 7

### 4-(2-Chloro-3-(4-(1,4-dimethyl-2-oxo-1,2-dihydropyridin-3-yl)-3-oxopiperazin-1-yl)phenyl)-4-cyanobutanoic acid (II-12h)

A mixture of II-12g (73 mg, 0.15 mmol), acetic acid (300 μL), concentrated sulfuric acid (40 μL) and acetonitrile (2 mL) was stirred at room temperature for 1 hour. The mixture was purified by prep-HPLC to give the target product II-12h (17 mg, 26%).

MS m/z (ESI): 443.1 [M+H⁺]

### Step 8

### 3-(2-Chloro-3-(4-(1,4-dimethyl-2-oxo-1,2-dihydropyridin-3-yl)-3-oxopiperazin-1-yl)phenyl) piperidine-2,6-dione (II-12)

A mixture of II-12h (17 mg, 0.038 mmol), concentrated sulfuric acid (30 μL) and acetic acid (300 μL) was stirred at 90° C. for 1 hour. The mixture was purified by prep-HPLC to give the target product II-12 (11 mg, 6%).

MS m/z (ESI): 443.1 [M+H⁺]

¹H NMR (400 MHz, DMSO-d₆) δ 10.89 (s, 1H), 7.63 (d, J=6.9 Hz, 1H), 7.33 (t, J=7.8 Hz, 1H), 7.24 (d, J=8.0 Hz, 1H), 7.08 (dd, J=7.6, 1.2 Hz, 1H), 6.19 (d, J=7.0 Hz, 1H), 4.29 (dd, J=11.9, 4.4 Hz, 1H), 3.87-3.66 (m, 3H), 3.48-3.38 (m, 5H), 3.32-3.26 (m, 1H), 2.83-2.73 (m, 1H), 2.58-2.53 (m, 1H), 2.35-2.23 (m, 1H), 2.09-1.95 (m, 4H).

### Example 13

### 3-(2-Chloro-3-(4-((5-cyclopropylpyridin-2-yl)methyl) piperazin-1-yl)phenyl) piperidine-2,6-dione (Compound II-13)

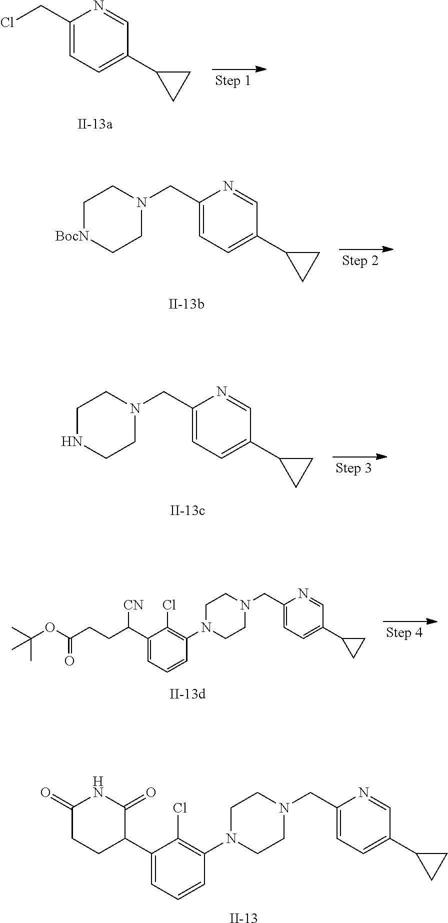

### Step 1

### tert-Butyl 4-((5-cyclopropylpyridin-2-yl)methyl) piperazine-1-carboxylate (II-13b)

To a solution of II-13a (600 mg, 3.6 mmol) and tert-butyl piperazine-1-carboxylate (1.0 g, 5.4 mmol) in DMF (10 mL) was added potassium carbonate (1.0 g, 7.2 mmol). The reaction mixture was stirred at 60° C. for 3 hours, added with water and extracted with ethyl acetate. The organic phase was concentrated under reduced pressure and the residue was purified by silica gel column chromatography (dichloromethane/methanol=100/0 to 95/5 v/v) to afford the target product II-13b (622 mg, 54%).

MS m/z (ESI): 318.2 [M+H⁺]

### Step 2

### 1-((5-Cyclopropylpyridin-2-yl)methyl) piperazine (II-13c)

To a solution of II-13b (622 mg, 1.9 mmol) in dichloromethane (6 mL) was added trifluoroacetic acid (2 mL). The reaction mixture was stirred at room temperature for 2 hours and concentrated under reduced pressure. The residue was dissolved in ethyl acetate, added dropwise to a sodium bicarbonate solution and extracted with ethyl acetate. The organic phase was concentrated under reduced pressure and the residue was purified by silica gel column chromatography (dichloromethane/methanol=100/0 to 93/7 v/v) to afford the target product II-13c (230 mg, 54%).

MS m/z (ESI): 218.1 [M+H⁺]

### Steps 3 to 4

### 3-(2-Chloro-3-(4-((5-cyclopropylpyridin-2-yl)methyl) piperazin-1-yl)phenyl) piperidine-2,6-dione (II-13)

II-13 was synthesized according to the procedures of Steps 5-6 for Example 3, but II-13c was used instead of II-2f and A-4 was used instead of II-2b.

MS m/z (ESI): 439.3 [M+H⁺]

¹H NMR (400 MHz, DMSO-d₆) δ 10.86 (s, 1H), 8.36 (s, 1H), 7.42 (d, J=7.4 Hz, 1H), 7.34 (d, J=8.0 Hz, 1H), 7.27 (t, J=7.8 Hz, 1H), 7.11 (d, J=7.1 Hz, 1H), 7.01 (d, J=7.5 Hz, 1H), 4.25 (dd, J=12.0, 5.0 Hz, 1H), 3.69 (brs, 2H), 3.10-2.88 (m, 4H), 2.84-2.59 (m, 5H), 2.58-2.53 (m, 1H), 2.35-2.20 (m, 1H), 2.04-1.90 (m, 2H), 1.04-0.95 (m, 2H), 0.78-0.69 (m, 2H).

### Example 14

### 3-(2-Chloro-3-(4-(1-methyl-2-oxo-1,2-dihydropyridin-3-yl) piperazin-1-yl)phenyl) piperidine-2,6-dione (Compound II-14)

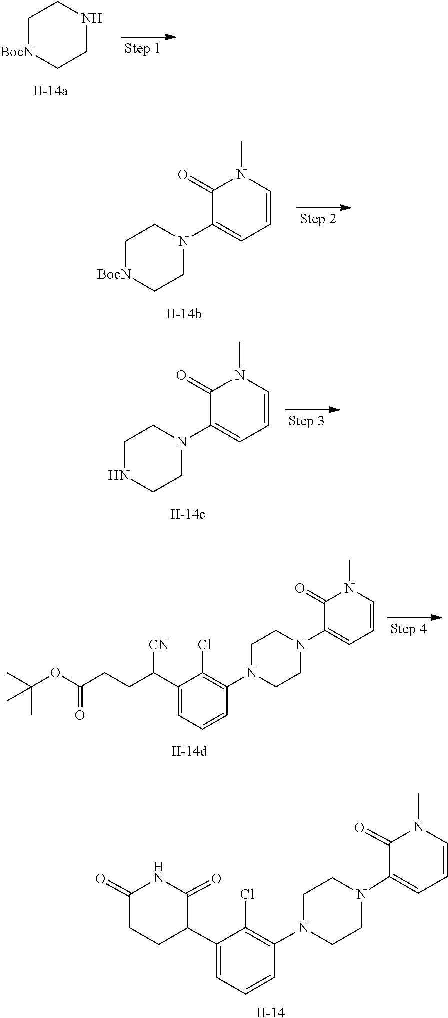

### Step 1

### tert-Butyl 4-(1-methyl-2-oxo-1,2-dihydropyridin-3-yl) piperazine-1-carboxylate (II-14b)

II-14b was synthesized according to the procedure of Step 5 for Example 3, but II-14a was used instead of II-2f and 3-bromo-1-methylpyridin-2 (1H)-one was used instead of II-2b.

MS m/z (ESI): 294.1 [M+H⁺]

### Step 2

### 1-Methyl-3-(piperazin-1-yl)pyridin-2 (1H)-one (II-14c)

A mixture of II-14b (0.60 g, 2.0 mmol) and a solution of HCl in 1,4-dioxane (4 M, 10 mL) was stirred at room temperature for 2 hours. The mixture was concentrated under reduced pressure to give the target product II-14c (600 mg, crude). This product was used directly in the next step without further purification.

MS m/z (ESI): 194.1 [M+H⁺]

### Steps 3 to 4

### 3-(2-Chloro-3-(4-(1-methyl-2-oxo-1,2-dihydropyridin-3-yl) piperazin-1-yl)phenyl) piperidine-2,6-dione (II-14)

II-14 was synthesized according to the procedures of Steps 5-6 for Example 3, but II-14c was used instead of II-2f and A-4 was used instead of II-2b.

MS m/z (ESI): 415.1 [M+H⁺]

¹H NMR (400 MHz, DMSO-d₆) δ 10.87 (s, 1H), 7.35 (d, J=6.1 Hz, 1H), 7.30 (t, J=7.8 Hz, 1H), 7.18 (d, J=7.9 Hz, 1H), 7.03 (d, J=7.6 Hz, 1H), 6.81 (d, J=6.5 Hz, 1H), 6.17 (t, J=7.0 Hz, 1H), 4.27 (dd, J=11.9, 5.0 Hz, 1H), 3.44 (s, 3H), 3.30-3.18 (m, 4H), 3.15-3.03 (m, 4H), 2.83-2.71 (m, 1H), 2.58-2.53 (m, 1H), 2.35-2.22 (m, 1H), 2.04-1.94 (m, 1H).

### Example 15

### 3-(2-Chloro-3-(4-((2-oxopyridin-1 (2H)-yl)methyl) piperidin-1-yl)phenyl) piperidine-2,6-dione (Compound II-15)

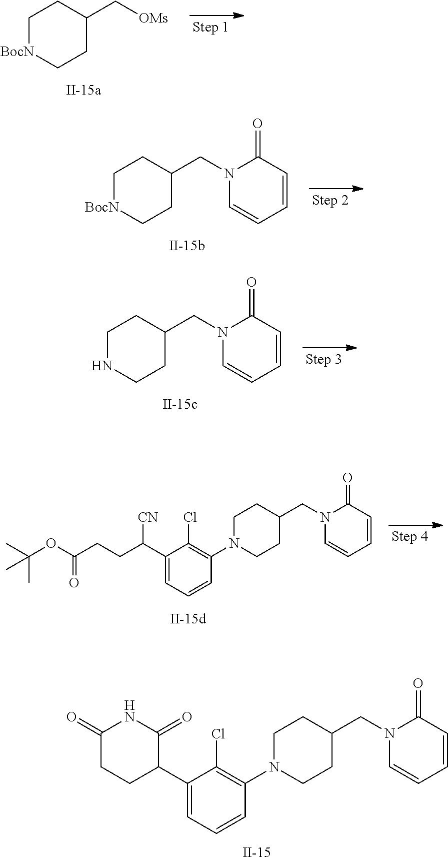

### Step 1

### tert-Butyl 4-((2-oxopyridin-1 (2H)-yl)methyl) piperidine-1-carboxylate (II-15b)

To a solution of pyridin-2 (1H)-one (325 mg, 3.4 mmol) in DMF (6 mL) was added sodium hydride (60%, 150 mg, 3.8 mmol). The mixture was stirred at 0° C. for 30 minutes, added with II-15a (1.0 g, 3.4 mmol) and stirred at 45° C. overnight. After quenching with water, the mixture was extracted with ethyl acetate. The organic phase was concentrated under reduced pressure and the residue was purified by silica gel column chromatography (petroleum ether/ethyl acetate=100/0 to 40/60 v/v) to afford the target product II-15b (547 mg, 54%).

MS m/z (ESI): 237.1 [M+H⁺-56]

### Step 2

### 1-(Piperidin-4-ylmethyl)pyridin-2 (1H)-one (II-15c)

II-15c was synthesized according to the procedure of Step 2 for Example 13, but II-15b was used instead of II-13b.

MS m/z (ESI): 193.1 [M+H⁺]

### Step 3

### tert-Butyl 4-(2-chloro-3-(4-((2-oxopyridin-1 (2H)-yl)methyl) piperidin-1-yl)phenyl)-4-cyanobutanoate (II-15d)

To a solution of A-4 (100 mg, 0.30 mmol) and II-15c (69 mg, 0.33 mmol) in 1,4-dioxane (5 mL) were added sodium tert-butoxide (55 mg, 0.60 mmol), Pd₂ (dba)₃ (42 mg, 0.045 mmol) and RuPhos (21 mg, 0.045 mmol). The reaction mixture was stirred at 105° C. overnight and concentrated under reduced pressure. The residue was purified by silica gel column chromatography (petroleum ether/ethyl acetate=100/0 to 30/70 v/v) to afford the target product II-15d (67 mg, 51%).

MS m/z (ESI): 470.3 [M+H⁺]

### Step 4

### 3-(2-Chloro-3-(4-((2-oxopyridin-1 (2H)-yl)methyl) piperidin-1-yl)phenyl) piperidine-2,6-dione (II-15)

II-15 was synthesized according to the procedure of Step 6 for Example 3, but II-15d was used instead of II-2g.

MS m/z (ESI): 414.2 [M+H⁺]

¹H NMR (400 MHz, DMSO-d₆) δ 10.86 (s, 1H), 7.68 (dd, J=6.7, 1.8 Hz, 1H), 7.44-7.37 (m, 1H), 7.25 (t, J=7.8 Hz, 1H), 7.08 (dd, J=8.0, 1.3 Hz, 1H), 6.98 (dd, J=7.7, 1.2 Hz, 1H), 6.38 (d, J=8.5 Hz, 1H), 6.20 (td, J=6.7, 1.3 Hz, 1H), 4.24 (dd, J=12.1, 5.0 Hz, 1H), 3.85 (d, J=7.3 Hz, 2H), 3.22 (t, J=11.6 Hz, 2H), 2.81-2.70 (m, 1H), 2.64-2.53 (m, 3H), 2.33-2.21 (m, 1H), 2.02-1.88 (m, 2H), 1.65-1.55 (m, 2H), 1.50-1.38 (m, 2H).

### Example 16

### 3-(2-Chloro-3-(8-(1-methyl-2-oxo-1,2-dihydropyridin-3-yl)-8-azabicyclo[3.2.1]octan-3-yl)phenyl) piperidine-2,6-dione (Compound II-16)

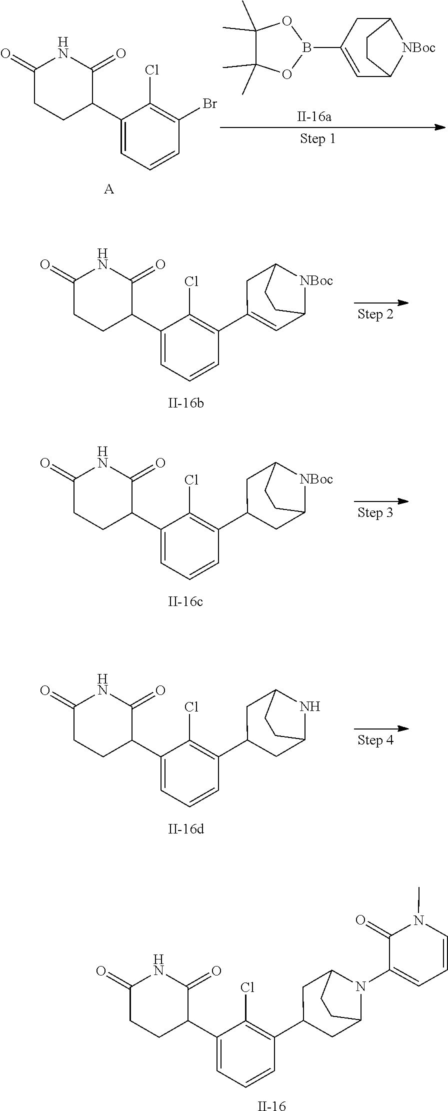

### Step 1

### tert-Butyl 3-(2-chloro-3-(2,6-dioxopiperidin-3-yl)phenyl)-8-azabicyclo[3.2.1]oct-2-ene-8-carboxylate (II-16b)

To a solution of A (500 mg, 1.7 mmol), II-16a (665 mg, 2.0 mmol), potassium phosphate (1 g, 5.0 mmol), and Pd(dppf)Cl₂ (60 mg, 0.083 mmol) in 1,4-dioxane (10 mL) was added water (1 mL). The mixture was stirred at 90° C. for 4 hours and concentrated under reduced pressure. The residue was purified by silica gel column chromatography (petroleum ether/ethyl acetate=3/1 v/v) to afford the target product II-16b (400 mg, 56%).

MS m/z (ESI): 331.2 [M+H⁺-100]

### Step 2

### tert-Butyl 3-(2-chloro-3-(2,6-dioxopiperidin-3-yl)phenyl)-8-azabicyclo[3.2.1]octane-8-carboxylate (II-16c)

To a solution of II-16b (400 mg, 0.93 mmol) in methanol (10 mL) was added palladium on carbon (10%, containing 55% water, 120 mg). The mixture was stirred under a hydrogen atmosphere at room temperature for 3 hours and filtered. The filtrate was concentrated under reduced pressure and the residue was purified by silica gel column chromatography (petroleum ether/ethyl acetate=3/1 v/v) to afford the target product II-16c (160 mg, 40%).

MS m/z (ESI): 333.1 [M+H⁺-100]

### Step 3

### 3-(3-(8-Azabicyclo[3.2.1]octan-3-yl)-2-chlorophenyl) piperidine-2,6-dione (II-16d)

A mixture of II-16c (160 mg, 0.37 mmol) and a solution of HCl in 1,4-dioxane (4 M, 5 mL) was stirred at room temperature for 2 hours. The mixture was concentrated under reduced pressure to give the target product II-16d as a hydrochloride salt (120 mg, 88%).

MS m/z (ESI): 333.1 [M+H⁺]

### Step 4

### 3-(2-Chloro-3-(8-(1-methyl-2-oxo-1,2-dihydropyridin-3-yl)-8-azabicyclo[3.2.1]octan-3-yl)phenyl) piperidine-2,6-dione (II-16)

To a mixture of II-16d (70 mg, 0.19 mmol), 3-iodo-1-methylpyridin-2 (1H)-one (45 mg, 0.19 mmol), copper (I) iodide (4 mg, 0.019 mmol) and DMSO (5 mL) were added L-proline (3 mg, 0.019 mmol) and potassium carbonate (79 mg, 0.57 mmol). The reaction mixture was stirred at 120° C. for 12 hours, added with water and extracted with ethyl acetate. The organic phase was concentrated under reduced pressure and the residue was purified by prep-HPLC to afford the target product II-16 (0.6 mg, 1%).

MS m/z (ESI): 440.1 [M+H⁺]

¹H NMR (400 MHz, DMSO-d₆) δ 10.87 (s, 1H), 7.25 (t, J=7.6 Hz, 1H), 7.19-7.09 (m, 3H), 6.71 (d, J=6.2 Hz, 1H), 6.13 (t, J=7.1 Hz, 1H), 4.72 (s, 2H), 4.26 (dd, J=12.1, 4.9 Hz, 1H), 3.43 (s, 1H), 2.81-2.70 (m, 1H), 2.59-2.53 (m, 1H), 2.32-2.21 (m, 1H), 2.04-1.93 (m, 3H), 1.88-1.77 (m, 4H), 1.59-1.48 (m, 2H).

### Example 17

### 3-(2-Chloro-3-(4-(1-cyclopropyl-2-oxo-1,2-dihydropyridin-3-yl) piperazin-1-yl)phenyl) piperidine-2,6-dione (Compound II-17)

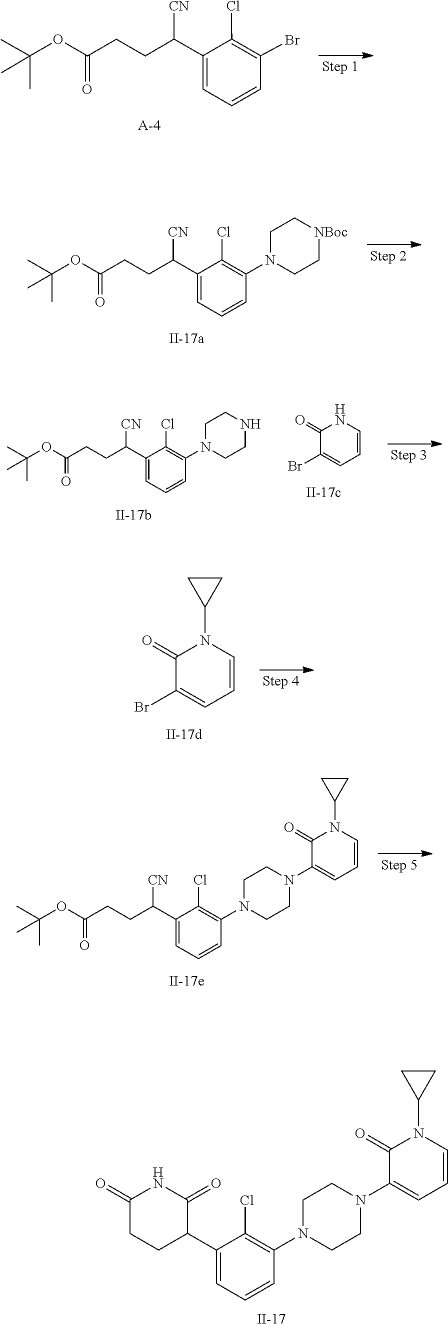

### Step 1

### tert-Butyl 4-(3-(4-(tert-butoxy)-1-cyano-4-oxobutyl)-2-chlorophenyl) piperazine-1-carboxylate (II-17a)

To a solution of A-4 (1.5 g, 4.2 mmol) and tert-butyl piperazine-1-carboxylate (1.2 g, 6.3 mmol) in 1,4-dioxane (20 mL) were added Pd₂ (dba)₃ (384 mg, 0.42 mmol), Xantphos (243 mg, 0.42 mmol) and cesium carbonate (4.1 g, 12.6 mmol). The reaction mixture was stirred at 100° C. overnight and concentrated under reduced pressure. The residue was purified by silica gel column chromatography (petroleum ether/ethyl acetate=91/9 v/v) to afford the target product II-17a (1.8 g, 90%).

MS m/z (ESI): 464.2 [M+H⁺]

### Step 2

### tert-Butyl 4-(2-chloro-3-(piperazin-1-yl)phenyl)-4-cyanobutanoate (II-17b)

To a solution of II-17a (1.8 g, 3.8 mmol) in dichloromethane (20 mL) was added trifluoroacetic acid (2.5 mL). The reaction mixture was stirred at room temperature for 2 hours, quenched with saturated aqueous sodium carbonate solution and extracted with ethyl acetate. The organic phase was dried over anhydrous sodium sulfate and concentrated under reduced pressure. The residue was purified by silica gel column chromatography (dichloromethane/methanol=94/6 v/v) to afford the target product II-17b (670 mg, 49%).

MS m/z (ESI): 364.2 [M+H⁺]

### Step 3

### 3-Bromo-1-cyclopropylpyridin-2 (1H)-one (II-17d)

To a solution of II-17c (2.0 g, 11.5 mmol) and cyclopropylboronic acid (1.8 g, 20.7 mmol) in 1,2-dichloroethane (40 mL) were added copper acetate (2.1 g, 11.5 mmol), sodium carbonate (2.4 g, 22.9 mmol) and 2,2′-bipyridine (1.8 g, 11.5 mmol). The reaction mixture was stirred at 70° C. in air for 4 hours and concentrated under reduced pressure. The residue was purified by silica gel column chromatography (dichloromethane) to afford the target product II-17d (1.1 g, 45%).

MS m/z (ESI): 213.9 [M+H⁺]

### Step 4

### tert-Butyl 4-(2-chloro-3-(4-(1-cyclopropyl-2-oxo-1,2-dihydropyridin-3-yl) piperazin-1-yl)phenyl)-4-cyanobutanoate (II-17e)

II-17e was synthesized according to the procedure of Step 3 for Example 15, but II-17b was used instead of II-15c and II-17d was used instead of A-4.

MS m/z (ESI): 497.2 [M+H⁺]

### Step 5

### 3-(2-Chloro-3-(4-(1-cyclopropyl-2-oxo-1,2-dihydropyridin-3-yl) piperazin-1-yl)phenyl) piperidine-2,6-dione (II-17)

II-17 was synthesized according to the procedure of Step 6 for Example 3, but II-17e was used instead of II-2g.

MS m/z (ESI): 441.2 [M+H⁺]

¹H NMR (400 MHz, DMSO-d₆) δ 10.87 (s, 1H), 7.30 (t, J=7.8 Hz, 1H), 7.23-7.14 (m, 2H), 7.03 (dd, J=7.6, 1.2 Hz, 1H), 6.75 (dd, J=7.3, 1.5 Hz, 1H), 6.13 (t, J=7.1 Hz, 1H), 4.27 (dd, J=12.0, 5.0 Hz, 1H), 3.36-3.33 (m, 1H), 3.28-3.16 (m, 4H), 3.15-3.04 (m, 4H), 2.81-2.71 (m, 1H), 2.58-2.53 (m, 1H), 2.34-2.23 (m, 1H), 2.05-1.95 (m, 1H), 1.02-0.95 (m, 2H), 0.83-0.76 (m, 2H).

### Example 18

### 3-(2-Chloro-3-(4-(1,5-dimethyl-2-oxo-1,2-dihydropyridin-3-yl) piperazin-1-yl)phenyl) piperidine-2,6-dione (Compound II-18)

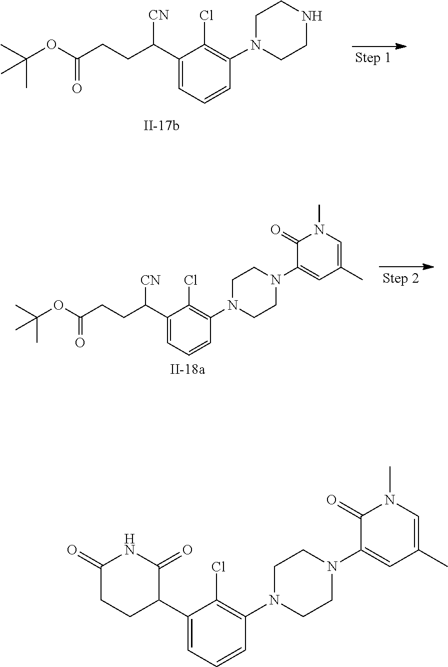 [SMILES: CC(C)(C)OC(=O)CCC(C#N)c1cccc(N2CCNCC2)c1Cl ; Cc1cc(N2CCN(c3cccc(C(C#N)CCC(=O)OC(C)(C)C)c3Cl)CC2)c(=O)n(C)c1 ; Cc1cc(N2CCN(c3cccc(C4CCC(=O)NC4=O)c3Cl)CC2)c(=O)n(C)c1]

### Step 1

### tert-Butyl 4-(2-chloro-3-(4-(1,5-dimethyl-2-oxo-1,2-dihydropyridin-3-yl) piperazin-1-yl)phenyl)-4-cyanobutanoate (II-18a)

II-18a was synthesized according to the procedure of Step 1 for Example 17, but II-17b was used instead of tert-butyl piperazine-1-carboxylate and 3-bromo-1,5-dimethylpyridin-2 (1H)-one was used instead of A-4.

MS m/z (ESI): 485.3 [M+H⁺]

### Step 2

### 3-(2-Chloro-3-(4-(1,5-dimethyl-2-oxo-1,2-dihydropyridin-3-yl) piperazin-1-yl)phenyl) piperidine-2,6-dione (II-18)

To a solution of II-18a (70 mg, 0.14 mmol) in acetic acid (2 mL) was added concentrated sulfuric acid (0.2 mL). The reaction mixture was stirred at 70° C. for 4 hours and concentrated under reduced pressure. The residue was purified by prep-HPLC to afford the target product II-18 (16.5 mg, 27%).

MS m/z (ESI): 429.2 [M+H⁺]

¹H NMR (400 MHz, DMSO-d₆) δ 10.87 (s, 1H), 7.29 (t, J=7.8 Hz, 1H), 7.18 (dd, J=8.0, 1.3 Hz, 1H), 7.12 (s, 1H), 7.02 (dd, J=7.6, 1.2 Hz, 1H), 6.65 (d, J=2.1 Hz, 1H), 4.27 (dd, J=12.0, 5.1 Hz, 1H), 3.39 (s, 3H), 3.28-3.17 (m, 4H), 3.13-3.02 (m, 4H), 2.83-2.70 (m, 1H), 2.58-2.53 (m, 1H), 2.36-2.22 (m, 1H), 2.08-1.95 (m, 4H).

Compound II-33 was synthesized according to the procedures for Example 18, but a different reactant was used instead of 3-bromo-1,5-dimethylpyridin-2 (1H)-one.

| | | Compound replacing 3- | |
|---|---|---|---|
| | | bromo-1,5- | |
| Compound | | dimethylpyridin-2(1H)- | MS m/z |
| Number | Compound Structure | one | (ESI) |
| II-33 | 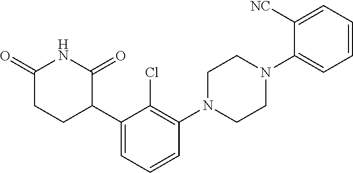 [SMILES: N#Cc1ccccc1N1CCN(c2cccc(C3CCC(=O)NC3=O)c2Cl)CC1] | 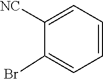 | 409.1 [M + H⁺] |

The NMR data for compound II-33 are as follows:

| Compound | ¹H NMR |
|---|---|
| 2-(4-(2-chloro-3-(2,6- | ¹H NMR (400 MHz, DMSO-d₆) δ 10.88 (s, 1H), 7.73 (dd, J = |
| dioxopiperidin-3- | 7.7, 1.4 Hz, 1H), 7.66-7.59 (m, 1H), 7.34-7.18 (m, 3H), |
| yl)phenyl)piperazin-1- | 7.13 (t, J = 7.5 Hz, 1H), 7.07-7.03 (m, 1H), 4.28 (dd, J = |
| yl)benzonitrile (II-33) | 12.0, 5.0 Hz, 1H), 3.42-3.31 (m, 4H), 3.22-3.09 (m, 4H), |
| | 2.84-2.71 (m, 1H), 2.62-2.53 (m, 1H), 2.35-2.23 (m, |
| | 1H), 2.05-1.96 (m, 1H). |

### Example 19

### 3-(2-Chloro-3-(6-(1-methyl-2-oxo-1,2-dihydropyridin-3-yl)-2,6-diazaspiro[3.3]heptan-2-yl)phenyl) piperidine-2,6-dione (Compound II-19)

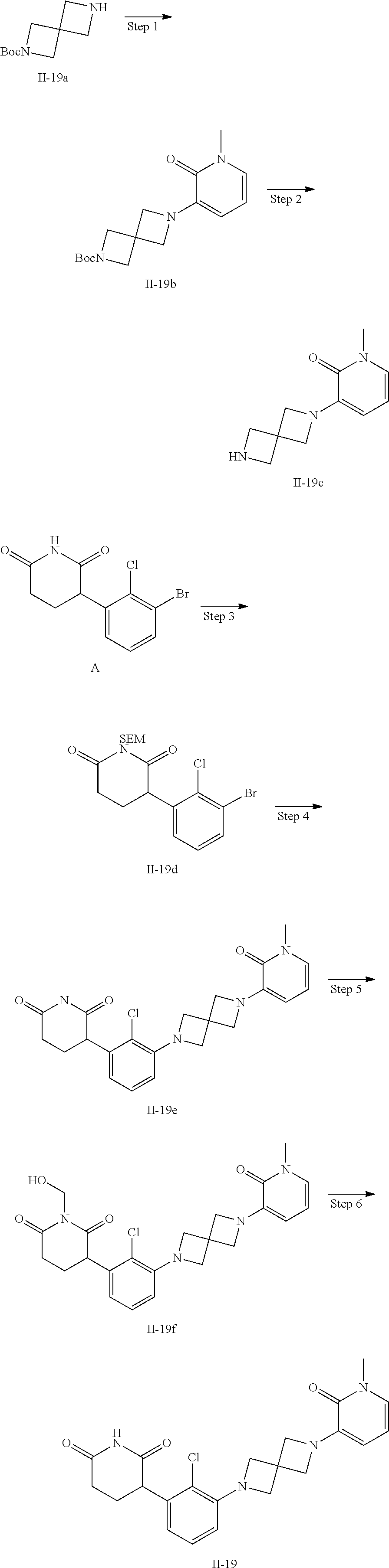

### Steps 1 to 2

### 1-Methyl-3-(2,6-diazaspiro[3.3]heptan-2-yl)pyridin-2 (1H)-one (II-19c)

II-19c was synthesized according to the procedures of Steps 1-2 for Example 17, but II-19a was used instead of tert-butyl piperazine-1-carboxylate and 3-bromo-1-methylpyridin-2 (1H)-one was used instead of A-4.

MS m/z (ESI): 206.2 [M+H⁺]

### Step 3

### 3-(3-Bromo-2-chlorophenyl)-1-((2-(trimethylsilyl) ethoxy)methyl) piperidine-2,6-dione (II-19d)

To a solution of A (1.0 g, 3.3 mmol) and DBU (1.0 g, 6.6 mmol) in THF (10 mL) at 0° C. was added SEM-C₁ (992 mg, 5.9 mmol). The reaction mixture was stirred at room temperature overnight, added with saturated sodium bicarbonate solution and extracted with ethyl acetate. The organic phase was concentrated under reduced pressure and the residue was purified by silica gel column chromatography (dichloromethane/methanol=100/0 to 90/10 v/v) to afford the target product II-19d (1.3 g, 91%).

### Step 4

### 3-(2-Chloro-3-(6-(1-methyl-2-oxo-1,2-dihydropyridin-3-yl)-2,6-diazaspiro[3.3]heptan-2-yl)phenyl)-1-((2-(trimethylsilyl) ethoxy)methyl) piperidine-2,6-dione (II-19e)

II-19e was synthesized according to the procedure of Step 5 for Example 3, but II-19c was used instead of II-2f and II-19d was used instead of II-2b.

MS m/z (ESI): 557.3 [M+H⁺]

### Step 5

### 3-(2-Chloro-3-(6-(1-methyl-2-oxo-1,2-dihydropyridin-3-yl)-2,6-diazaspiro[3.3]heptan-2-yl)phenyl)-1-(hydroxymethyl) piperidine-2,6-dione (II-19f)

A mixture of II-19e (46 mg, 0.082 mmol), trifluoroacetic acid (200 μL) and dichloromethane (1 mL) was stirred at room temperature for 1 hour. The mixture was concentrated under reduced pressure to give the target product II-19f (crude product, 49 mg).

MS m/z (ESI): 457.2 [M+H⁺]

### Step 6

### 3-(2-Chloro-3-(6-(1-methyl-2-oxo-1,2-dihydropyridin-3-yl)-2,6-diazaspiro[3.3]heptan-2-yl)phenyl) piperidine-2,6-dione (II-19)

A mixture of II-19f (crude product, 49 mg, 0.082 mmol), ammonia water (200 μL) and acetonitrile (1 mL) was stirred at room temperature for 1 hour. The mixture was concentrated under reduced pressure and the residue was purified by prep-HPLC to afford the target product II-19 (8.0 mg, 23%).

MS m/z (ESI): 427.2 [M+H⁺]

¹H NMR (400 MHz, DMSO-d₆) δ 10.84 (s, 1H), 7.14 (t, J=7.9 Hz, 1H), 7.05 (dd, J=6.7, 1.6 Hz, 1H), 6.73-6.67 (m, 1H), 6.57 (d, J=7.1 Hz, 1H), 6.19 (dd, J=7.2, 1.6 Hz, 1H), 6.08 (t, J=6.9 Hz, 1H), 4.23-4.09 (m, 5H), 4.01 (s, 4H), 3.38 (s, 3H), 2.78-2.68 (m, 1H), 2.48-2.39 (m, 1H), 2.29-2.16 (m, 1H), 2.02-1.92 (m, 1H).

### Example 20

### 3-(2-Chloro-3-(4-(1,6-dimethyl-2-oxo-1,2-dihydropyridin-3-yl) piperazin-1-yl)phenyl) piperidine-2,6-dione (Compound II-20)

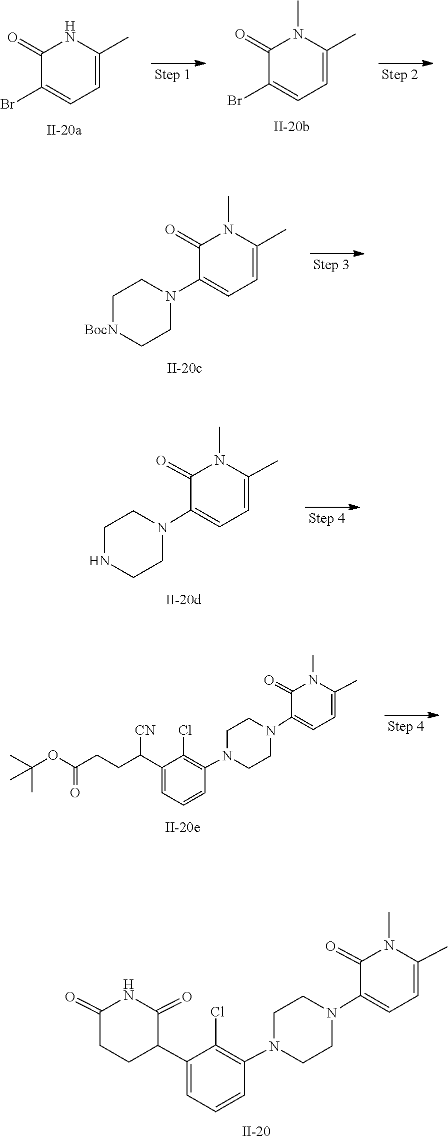

### Step 1

### 3-Bromo-1,6-dimethylpyridin-2 (1H)-one (II-20b)

To a mixture of 3-bromo-6-methylpyridin-2 (1H)-one II-20a (1.0 g, 5.3 mmol) and THF (15 mL) at 0° C. was added sodium hydride (60%, 255 mg, 6.4 mmol). The mixture was stirred at room temperature for 0.5 hour, added with lithium bromide (926 mg, 11 mmol) and stirred at room temperature for 1 hour. The mixture was then added with methyl iodide (1.5 g, 11 mmol), stirred overnight, diluted with dichloromethane, and washed with aqueous sodium hydroxide solution (1 M). The organic phase was concentrated under reduced pressure to afford the target product II-20b (900 mg, 84%).

MS m/z (ESI): 202.0 [M+H⁺]

### Step 2

### tert-Butyl 4-(1,6-dimethyl-2-oxo-1,2-dihydropyridin-3-yl) piperazine-1-carboxylate (II-20c)

To a mixture of II-20b (760 mg, 3.8 mmol), tert-butyl piperazine-1-carboxylate (1.1 g, 5.7 mmol), sodium tert-butoxide (1.1 g, 11.4 mmol) and 1,4-dioxane (10 mL) were added Xantphos (219 mg, 0.38 mmol) and Pd₂(dba)₃ (346 mg, 0.38 mmol). The reaction mixture was stirred at 100° C. for 6 hours and concentrated under reduced pressure. The residue was purified by silica gel column chromatography (dichloromethane/methanol=100/0 to 98/2 v/v) to afford the target product II-20c (460 mg, 40%).

MS m/z (ESI): 308.2 [M+H⁺]

### Step 3

### 1,6-Dimethyl-3-(piperazin-1-yl)pyridin-2 (1H)-one (II-20d)

A mixture of II-20c (460 mg, 1.5 mmol) and a solution of HCl in 1,4-dioxane (4 M, 10 mL) was stirred at room temperature for 1 hour. The mixture was concentrated under reduced pressure and the residue was triturated with acetonitrile to give the target product II-20d as a hydrochloride salt (350 mg, 96%).

MS m/z (ESI): 208.2 [M+H⁺]

### Step 4

### tert-Butyl 4-(2-chloro-3-(4-(1,6-dimethyl-2-oxo-1,2-dihydropyridin-3-yl) piperazin-1-yl)phenyl)-4-cyanobutanoate (II-20e)

To a mixture of A-4 (100 mg, 0.28 mmol), II-20d (82 mg, 0.34 mmol), sodium tert-butoxide (81 mg, 0.84 mmol) and 1,4-dioxane (5 mL) were added Xantphos (16 mg, 0.028 mmol) and Pd₂(dba)₃ (26 mg, 0.028 mmol). The mixture was stirred at 100° C. for 6 hours and concentrated under reduced pressure. The residue was purified by silica gel column chromatography (dichloromethane/methanol=100/0 to 97/3 v/v) to afford the target product II-20e (100 mg, 74%).

MS m/z (ESI): 485.2 [M+H⁺]

### Step 5

### 3-(2-Chloro-3-(4-(1,6-dimethyl-2-oxo-1,2-dihydropyridin-3-yl) piperazin-1-yl)phenyl) piperidine-2,6-dione (II-20)

II-20 was synthesized according to the procedure of Step 6 for Example 3, but II-20e was used instead of II-2g.

MS m/z (ESI): 429.1 [M+H⁺]

¹H NMR (400 MHz, DMSO-d₆) δ 10.87 (s, 1H), 7.30 (t, J=7.8 Hz, 1H), 7.23-7.16 (m, 1H), 7.04-6.98 (m, 1H), 6.79 (d, J=7.6 Hz, 1H), 6.09 (d, J=7.7 Hz, 1H), 4.27 (dd, J=12.0, 5.0 Hz, 1H), 3.89-3.52 (m, 4H), 3.44 (s, 3H), 3.15-3.06 (m, 4H), 2.83-2.70 (m, 1H), 2.61-2.53 (m, 1H), 2.35-2.21 (m, 4H), 2.05-1.96 (m, 1H).

### Example 21

### 3-(2-Chloro-3-(3-methyl-4-(1-methyl-2-oxo-1,2-dihydropyridin-3-yl) piperazin-1-yl)phenyl) piperidine-2,6-dione (Compound II-21)

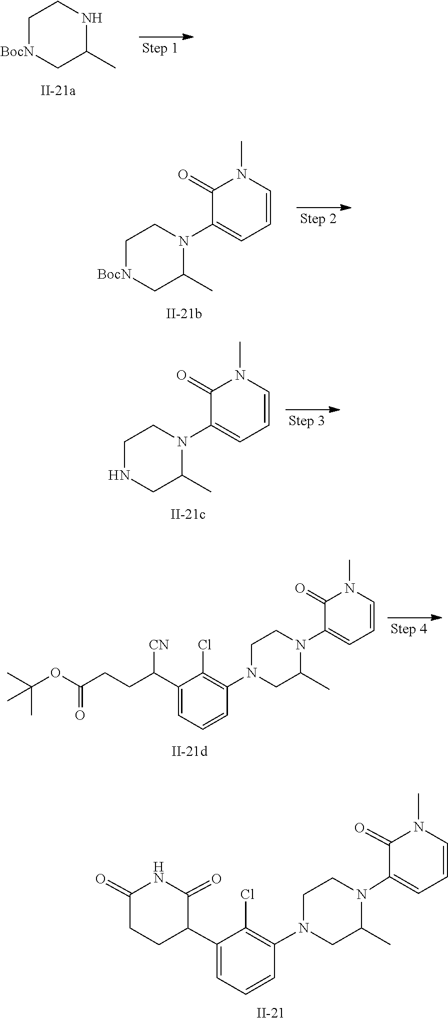

### Step 1

### tert-Butyl 3-methyl-4-(1-methyl-2-oxo-1,2-dihydropyridin-3-yl) piperazine-1-carboxylate (II-21b)

To a mixture of tert-butyl 3-methylpiperazine-1-carboxylate II-21a (500 mg, 2.5 mmol) and 1,4-dioxane (10 mL) were added 3-bromo-1-methylpyridin-2 (1H)-one (935 mg, 5.0 mmol), RuPhos-Pd-G3 (209 mg, 0.25 mmol) and sodium tert-butoxide (720 mg, 7.5 mmol). The mixture was stirred at 140° C. for 14 hours and concentrated under reduced pressure. The residue was purified by silica gel column chromatography (dichloromethane/methanol=60/1 to 20/1 v/v) to afford the target product II-21b (150 mg, 20%).

MS m/z (ESI): 308.2 [M+H⁺]

### Step 2

### 1-Methyl-3-(2-methylpiperazin-1-yl)pyridin-2 (1H)-one (II-21c)

A mixture of II-21b (60 mg, 0.20 mmol) and a solution of HCl in 1,4-dioxane (4 M, 5 mL) was stirred at room temperature for 1 hour. The mixture was concentrated under reduced pressure to give the target product II-21c as a hydrochloride salt (50 mg, crude). The product was used directly in the next step without further purification.

MS m/z (ESI): 208.1 [M+H⁺]

### Step 3

### tert-Butyl 4-(2-chloro-3-(3-methyl-4-(1-methyl-2-oxo-1,2-dihydropyridin-3-yl) piperazin-1-yl)phenyl)-4-cyanobutanoate (II-21d)

To a mixture of II-21c (crude product, 50 mg) and 1,4-dioxane (5 mL) were added A-4 (110 mg, 0.31 mmol), RuPhos-Pd-G3 (18 mg, 0.021 mmol) and sodium tert-butoxide (59 mg, 0.62 mmol). The reaction mixture was stirred at 100° C. for 2 hours and concentrated under reduced pressure. The residue was purified by silica gel column chromatography (dichloromethane/methanol=60/1 to 20/1 v/v) to afford the target product II-21d (53 mg, 52% for two steps).

MS m/z (ESI): 485.2 [M+H⁺]

### Step 4

### 3-(2-Chloro-3-(3-methyl-4-(1-methyl-2-oxo-1,2-dihydropyridin-3-yl) piperazin-1-yl)phenyl) piperidine-2,6-dione (II-21)

II-21 was synthesized according to the procedure of Step 6 for Example 3, but II-21d was used instead of II-2g.

MS m/z (ESI): 429.1 [M+H⁺]

¹H NMR (400 MHz, DMSO-d₆) δ 10.87 (s, 1H), 7.38-7.27 (m, 2H), 7.16 (d, J=7.7 Hz, 1H), 7.03 (d, J=7.6 Hz, 1H), 6.77 (d, J=7.1 Hz, 1H), 6.16 (t, J=7.0 Hz, 1H), 4.56-4.47 (m, 1H), 4.26 (dd, J=12.0, 5.0 Hz, 1H), 3.43 (s, 3H), 3.23-3.14 (m, 1H), 3.12-2.89 (m, 4H), 2.83-2.70 (m, 1H), 2.35-2.22 (m, 1H), 2.04-1.93 (m, 1H), 1.19-1.10 (m, 3H).

Compound II-22 was synthesized according to the procedures for Example 21, but a different reactant was used instead of II-21a.

| Compound | | Compound | MS m/z |
|---|---|---|---|
| Number | Compound Structure | replacing II-21a | (ESI) |
| II-22 | 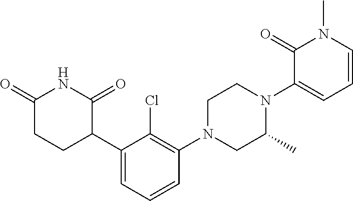 [SMILES: C[C@@H]1CN(c2cccc(C3CCC(=O)NC3=O)c2Cl)CCN1c1cccn(C)c1=O] | 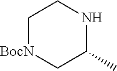 | 429.1 [M + H⁺] |

The NMR data for compound II-22 are as follows:

| Compound | ¹H NMR |
|---|---|
| 3-(2-chloro-3-((R)-3- | ¹H NMR (400 MHz, DMSO-d₆) δ 10.87 (s, 1H), 7.38-7.26 |
| methyl-4-(1-methyl-2- | (m, 2H), 7.16 (d, J = 7.7 Hz, 1H), 7.03 (d, J = 7.6 Hz, 1H), |
| oxo-1,2- | 6.78 (d, J = 6.9 Hz, 1H), 6.16 (t, J = 7.0 Hz, 1H), 4.58-4.46 |
| dihydropyridin-3- | (m, 1H), 4.26 (dd, J = 12.0, 5.0 Hz, 1H), 3.43 (s, 3H), 3.41- |
| yl)piperazin-1- | 3.36 (m, 1H), 3.23-3.13 (m, 1H), 3.10-2.89 (m, 4H), |
| yl)phenyl)piperidine- | 2.82-2.71 (m, 1H), 2.61-2.53 (m, 1H), 2.36-2.21 (m, |
| 2,6-dione (II-22) | 1H), 2.05-1.93 (m, 1H), 1.04 (dd, J = 6.5, 1.9 Hz, 3H). |

### Example 22

### 3-(2-Chloro-3-((S)-3-methyl-4-(1-methyl-2-oxo-1,2-dihydropyridin-3-yl) piperazin-1-yl)phenyl) piperidine-2,6-dione (Compound II-23)

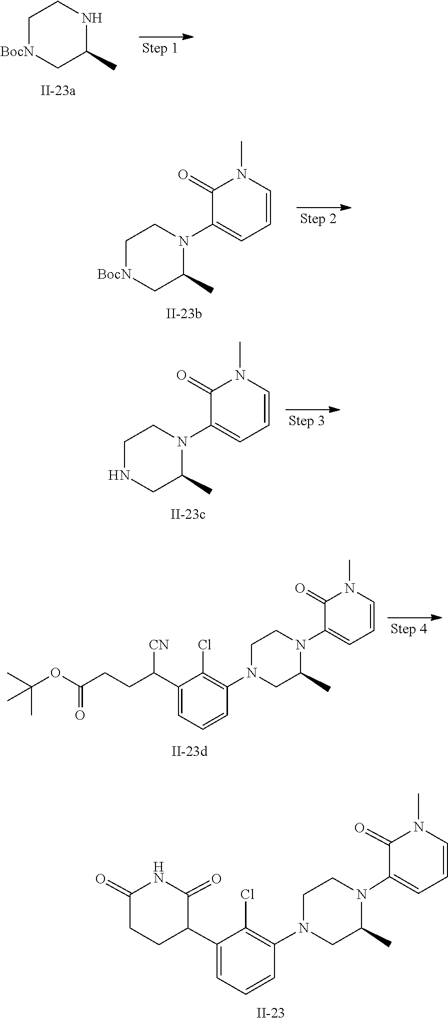

### Steps 1 to 2

### (S)-1-Methyl-3-(2-methylpiperazin-1-yl)pyridin-2 (1H)-one (II-23c)

II-23c was synthesized according to the procedures of Steps 1-2 for Example 21, but II-23a was used instead of II-21a.

MS m/z (ESI): 208.1 [M+H⁺]

### Step 3

### tert-Butyl 4-(2-chloro-3-((S)-3-methyl-4-(1-methyl-2-oxo-1,2-dihydropyridin-3-yl) piperazin-1-yl)phenyl)-4-cyanobutanoate (II-23d)

To a mixture of II-23c (crude product, 220 mg) and 1,4-dioxane (10 mL) were added with A-4 (456 mg, 1.3 mmol), Pd₂ (dba)₃ (156 mg, 0.17 mmol), Ruphos (79 mg, 0.17 mmol) and sodium tert-butoxide (204 mg, 2.2 mmol). The reaction mixture was stirred at 105° C. overnight. The residue was concentrated under reduced pressure, and the residue was purified by silica gel column chromatography (dichloromethane/methanol=100/0 to 95/5 v/v) to give the desired product II-23d (300 mg, 58%).

MS m/z (ESI): 485.3 [M+H⁺]

### Step 4

### 3-(2-Chloro-3-((S)-3-methyl-4-(1-methyl-2-oxo-1,2-dihydropyridin-3-yl) piperazin-1-yl)phenyl) piperidine-2,6-dione (II-23)

II-23 was synthesized according to the procedure of Step 6 for Example 3, but II-23d was used instead of II-2g.

MS m/z (ESI): 429.1 [M+H⁺]

¹H NMR (400 MHz, DMSO-d₆) δ 10.87 (s, 1H), 7.36-7.24 (m, 2H), 7.16 (d, J=7.7 Hz, 1H), 7.02 (d, J=7.6 Hz, 1H), 6.77 (d, J=7.1 Hz, 1H), 6.16 (t, J=7.0 Hz, 1H), 4.57-4.46 (m, 1H), 4.26 (dd, J=12.0, 5.0 Hz, 1H), 3.43 (s, 3H), 3.38-3.33 (m, 1H), 3.24-3.13 (m, 1H), 3.10-2.88 (m, 4H), 2.82-2.71 (m, 1H), 2.60-2.53 (m, 1H), 2.36-2.21 (m, 1H), 2.06-1.94 (m, 1H), 1.04 (dd, J=6.5, 1.9 Hz, 3H).

### Example 23

### 3-(2-Chloro-3-(1-(6-cyclopropylpyridin-3-yl) piperidin-4-yl)phenyl) piperidine-2,6-dione (Compound II-24)

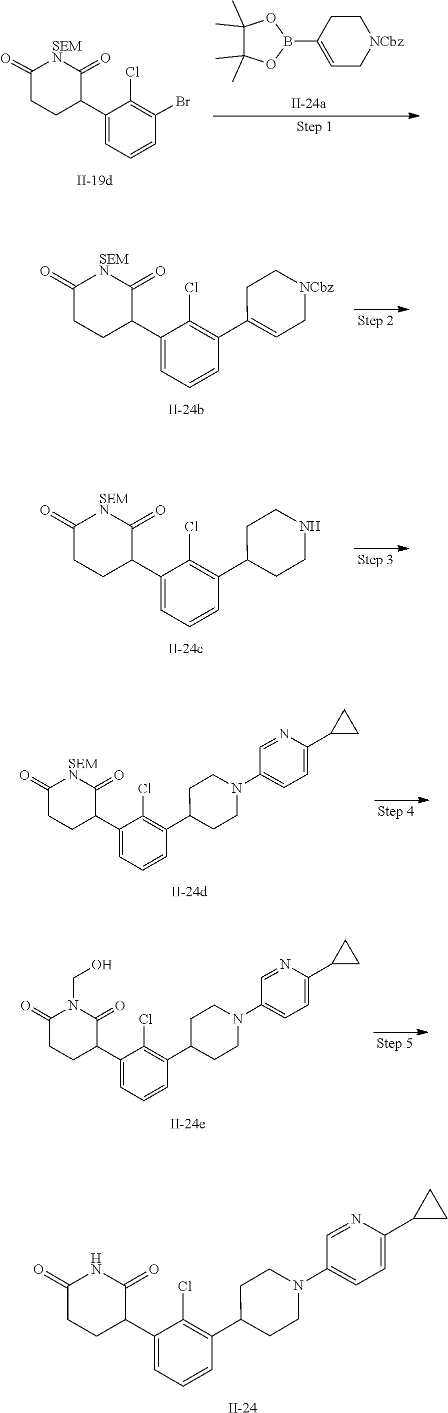

### Step 1

### Benzyl 4-(2-chloro-3-(2,6-dioxo-1-((2-(trimethylsilyl) ethoxy)methyl) piperidin-3-yl)phenyl)-3,6-dihydropyridine-1 (2H)-carboxylate (II-24b)

A mixture of II-19d (2.1 g, 4.9 mmol), II-24a (2.5 g, 7.3 mmol), potassium phosphate (3.1 g, 14.7 mmol), Pd(dppf)Cl₂ (358 mg, 0.49 mmol), 1,4-dioxane (21 mL) and water (7 mL) was stirred at 100° C. overnight. The mixture was cooled to room temperature, added with water, and extracted with ethyl acetate. The organic phase was concentrated under reduced pressure, and the residue was purified by silica gel column chromatography (petroleum ether/ethyl acetate=65/35 v/v) to give the target product II-24b (1.3 g, 65%).

MS m/z (ESI): 491.2 [M+Na⁺−100]

### Step 2

### 3-(2-Chloro-3-(piperidin-4-yl)phenyl)-1-((2-(trimethylsilyl) ethoxy)methyl) piperidine-2,6-dione (II-24c)

A mixture of II-24b (520 mg, 0.91 mmol), platinum dioxide (110 mg), and ethyl acetate (10 mL) was stirred at room temperature under a hydrogen atmosphere for 4 hours. The mixture was filtered, and the filtrate was concentrated under reduced pressure to give the target product II-24c (420 mg, 100%). The product was used directly in the next step without further purification.

### Step 3

### 3-(2-Chloro-3-(1-(6-cyclopropylpyridin-3-yl) piperidin-4-yl)phenyl)-1-((2-(trimethylsilyl) ethoxy)methyl) piperidine-2,6-dione (II-24d)

To a solution of II-24c (420 mg, 0.91 mmol) and 5-bromo-2-cyclopropylpyridine (360 mg, 1.8 mmol) in 1,4-dioxane (20 mL) were added Pd₂ (dba)₃ (83 mg, 0.091 mmol), Xantphos (53 mg, 0.091 mmol) and cesium carbonate (889 mg, 2.73 mmol). The reaction mixture was stirred at 100° C. for 48 hours. The mixture was cooled to room temperature, added with water, and extracted with ethyl acetate. The organic phase was concentrated under reduced pressure, and the residue was purified by silica gel column chromatography (dichloromethane/methanol=96/4 v/v) to give the target product II-24d (110 mg, 22%).

MS m/z (ESI): 554.3 [M+H⁺]

### Steps 4 to 5

### 3-(2-Chloro-3-(1-(6-cyclopropylpyridin-3-yl) piperidin-4-yl)phenyl) piperidine-2,6-dione (II-24)

II-24 was synthesized according to the procedures of Steps 5-6 for Example 19, but II-24d was used instead of II-19e.

MS m/z (ESI): 424.2 [M+H⁺]

¹H NMR (400 MHz, DMSO-d₆) δ 10.88 (s, 1H), 8.16 (d, J=2.8 Hz, 1H), 7.37-7.25 (m, 3H), 7.20 (dd, J=7.3, 1.7 Hz, 1H), 7.11 (d, J=8.5 Hz, 1H), 4.29 (dd, J=12.1, 5.1 Hz, 1H), 3.79 (d, J=11.2 Hz, 2H), 3.19-3.08 (m, 1H), 2.83-2.71 (m, 3H), 2.62-2.53 (m, 1H), 2.32-2.22 (m, 1H), 2.05-1.94 (m, 2H), 1.93-1.70 (m, 4H), 0.89-0.75 (m, 4H).

### Example 24

### 3-(2-Chloro-3-(3-methyl-4-(1-(methyl-d₃)-2-oxo-1,2-dihydropyridin-3-yl) piperazin-1-yl)phenyl) piperidine-2,6-dione (Compound II-25)

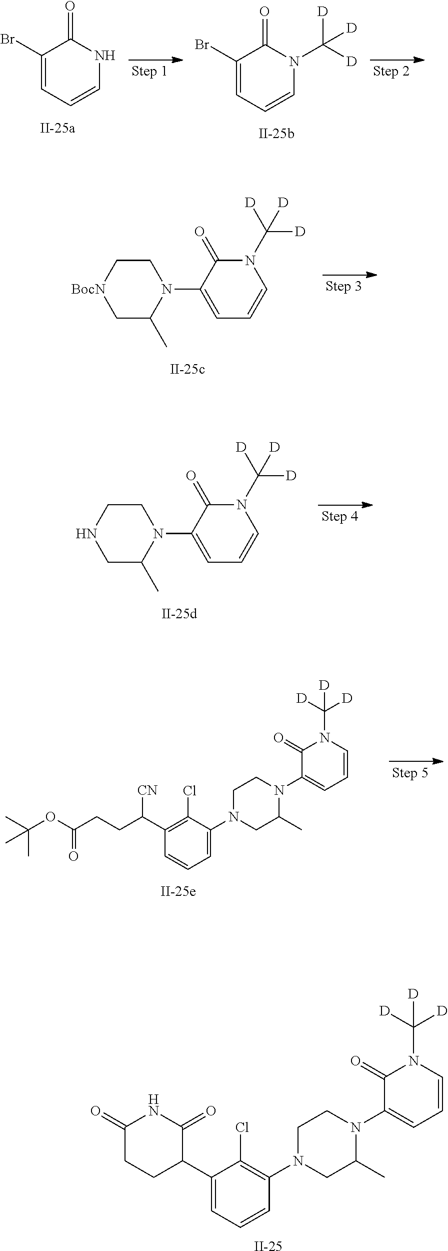

### Step 1

### 3-Bromo-1-(methyl-d₃)pyridin-2 (1H)-one (II-25b)

To a mixture of II-25a (3.0 g, 17.2 mmol), K₂CO₃ (7.1 g, 51.7 mmol) and DMF (45 mL) was add deuterated iodomethane (3.7 g, 25.9 mmol), and the reaction mixture was stirred at room temperature for 3 hours. The mixture was diluted with water and extracted with ethyl acetate. The organic phase was concentrated under reduced pressure, and the residue was purified by column chromatography on silica gel (petroleum ether/ethyl acetate=85/15 v/v) to give the target product II-25b (1.9 g, 58%).

MS m/z (ESI): 191.0 [M+H⁺]

### Step 2

### tert-Butyl 3-methyl-4-(1-(methyl-d₃)-2-oxo-1,2-dihydropyridin-3-yl) piperazine-1-carboxylate (II-25c)

To a mixture of II-25b (1.9 g, 9.8 mmol), tert-butyl 3-methylpiperazine-1-carboxylate (2.4 g, 11.8 mmol), sodium tert-butoxide (2.8 g, 29.5 mmol) and 1,4-dioxane (25 mL) were add Ruphos (460 mg, 0.99 mmol) and Pd₂(dba)₃ (902 mg, 0.99 mmol), and the reaction mixture was stirred at 100° C. for 3 hours. The mixture was concentrated under reduced pressure and the residue was purified by silica gel column chromatography (dichloromethane/methanol=100/0 to 97/3 v/v) to give the target product II-25c (2.0 g, 65%).

MS m/z (ESI): 311.2 [M+H⁺]

### Steps 3 to 5

### 3-(2-Chloro-3-(3-methyl-4-(1-(methyl-d₃)-2-oxo-1,2-dihydropyridin-3-yl) piperazin-1-yl)phenyl) piperidine-2,6-dione (II-25)

II-25 was synthesized according to the procedures of Steps 2-4 for Example 21, but II-25c was used instead of II-21b.

MS m/z (ESI): 432.2 [M+H⁺]

¹H NMR (400 MHz, DMSO-d₆) δ 10.87 (s, 1H), 7.38-7.24 (m, 2H), 7.16 (d, J=7.8 Hz, 1H), 7.03 (d, J=7.6 Hz, 1H), 6.77 (d, J=7.0 Hz, 1H), 6.16 (t, J=7.0 Hz, 1H), 4.61-4.42 (m, 1H), 4.27 (dd, J=11.9, 4.9 Hz, 1H), 3.41-3.35 (m, 1H), 3.22-3.12 (m, 1H), 3.11-2.87 (m, 4H), 2.82-2.70 (m, 1H), 2.61-2.53 (m, 1H), 2.36-2.22 (m, 1H), 2.06-1.94 (m, 1H), 1.10-0.97 (m, 3H).

### Example 25

### 3-(2-Chloro-3-(2-methyl-4-(1-methyl-2-oxo-1,2-dihydropyridin-3-yl) piperazin-1-yl)phenyl) piperidine-2,6-dione (Compound II-26)

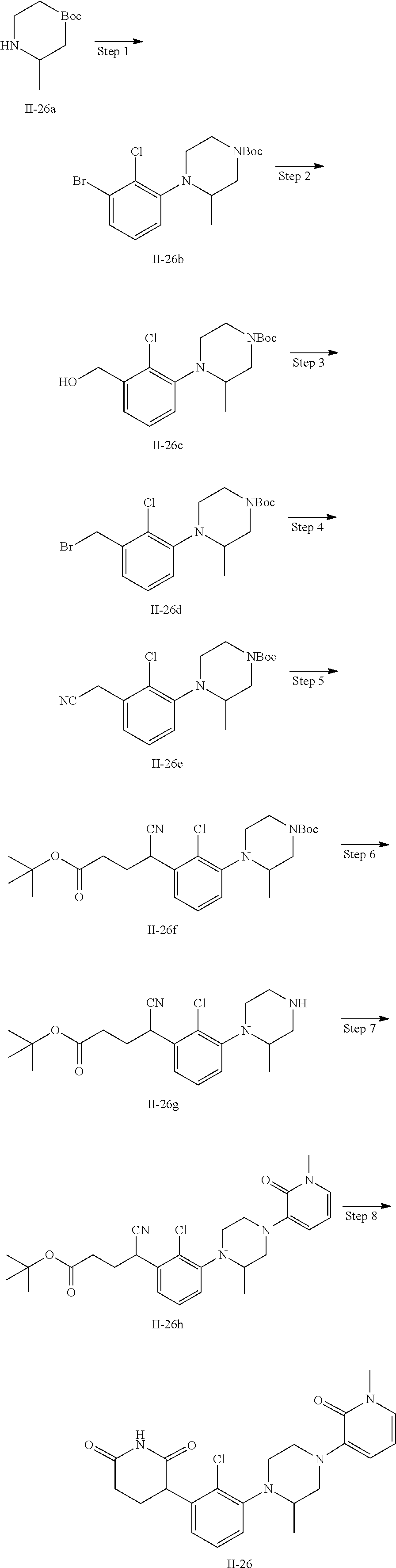

### Step 1

### tert-Butyl 4-(3-bromo-2-chlorophenyl)-3-methylpiperazine-1-carboxylate (II-26b)

To a solution of 1,3-dibromo-2-chlorobenzene (9.9 g, 37 mmol) and II-26a (5 g, 25 mmol) in 1,4-dioxane (90 mL) were add cesium carbonate (24.1 g, 73.9 mmol), Pd₂ (dba)₃ (2.3 g, 2.5 mmol) and Xantphos (1.5 g, 2.6 mmol), and the reaction mixture was stirred at 100° C. overnight. After filtration, the filtrate was concentrated under reduced pressure, and the residue was purified by silica gel column chromatography (petroleum ether/ethyl acetate=95/5 v/v), followed by further purification by prep-HPLC to give the target product II-26b (776 mg, 8%).

MS m/z (ESI): 389.0 [M+H⁺]

### Step 2

### tert-Butyl 4-(2-chloro-3-(hydroxymethyl)phenyl)-3-methylpiperazine-1-carboxylate (II-26c)

To a solution of II-26b (776 mg, 2.0 mmol) and Pd(PPh₃)₄ (180 mg, 0.16 mmol) in 1,4-dioxane (10 mL) was added tributyltin methanol (1.3 g, 4.0 mmol) and the reaction mixture was stirred at 110° C. overnight. The mixture was concentrated under reduced pressure, and the residue was purified by silica gel column chromatography (petroleum ether/ethyl acetate=81/19 v/v) to give the target product II-26c (660 mg, 97%).

MS m/z (ESI): 341.1 [M+H⁺]

### Step 3

### tert-Butyl 4-(3-(bromomethyl)-2-chlorophenyl)-3-methylpiperazine-1-carboxylate (II-26d)

To a solution of II-26c (590 mg, 1.7 mmol) and triphenylphosphine (500 mg, 1.9 mmol) in dichloromethane (10 mL) at 0° C. was added carbon tetrabromide (630 mg, 1.9 mmol), and the reaction mixture was stirred at room temperature for 2 hours. The mixture was concentrated under reduced pressure, and the residue was purified by silica gel column chromatography (petroleum ether/ethyl acetate=94/6 v/v) to give the target product II-26d (325 mg, 46%).

MS m/z (ESI): 403.0 [M+H⁺]

### Step 4

### tert-Butyl 4-(2-chloro-3-(cyanomethyl)phenyl)-3-methylpiperazine-1-carboxylate (II-26e)

To a solution of II-26d (325 mg, 0.80 mmol) and TMSCN (120 mg, 1.2 mmol) in dichloromethane (6 mL) at 0° C. was added dropwise a solution of TBAF in THF (1 M, 1.2 mL, 1.2 mmol), and the reaction mixture was stirred at room temperature for 2 hours. The mixture was concentrated under reduced pressure, and the residue was purified by silica gel column chromatography (petroleum ether/ethyl acetate=91/9 v/v) to give the target product II-26e (245 mg, 87%).

MS m/z (ESI): 350.1 [M+H⁺]

### Step 5

### tert-Butyl 4-(3-(4-(tert-butoxy)-1-cyano-4-oxobutyl)-2-chlorophenyl)-3-methylpiperazine-1-carboxylate (II-26f)

To a solution of II-26e (245 mg, 0.70 mmol) and sodium methoxide (10 mg, 0.19 mmol) in THF (5 mL) at 0° C. was added dropwise tert-butyl acrylate (90 mg, 0.7 mmol), and the reaction mixture was stirred at room temperature for 2 hours. The reaction was quenched with water and extracted with ethyl acetate. The organic phase was dried over anhydrous sodium sulfate and concentrate under reduced pressure. The residue was purified by silica gel column chromatography (petroleum ether/ethyl acetate=93/7 v/v) to give the target product II-26f (268 mg, 80%).

MS m/z (ESI): 478.2 [M+H⁺]

### Steps 6 to 8

### 3-(2-Chloro-3-(2-methyl-4-(1-methyl-2-oxo-1,2-dihydropyridin-3-yl) piperazin-1-yl)phenyl) piperidine-2,6-dione (II-26)

II-26 was synthesized according to the procedures of Steps 4-6 for Example 3, but II-26f was used instead of II-2e and 3-bromo-1-methylpyridin-2 (1H)-one was used instead of II-2b.

MS m/z (ESI): 429.1 [M+H⁺]

¹H NMR (400 MHz, DMSO-d₆) δ 10.87 (s, 1H), 7.38-7.22 (m, 3H), 7.07 (d, J=7.3 Hz, 1H), 6.79-6.73 (m, 1H), 6.16 (t, J=7.0 Hz, 1H), 4.27 (dd, J=11.9, 4.8 Hz, 1H), 3.62-3.36 (m, 6H), 3.23-3.15 (m, 1H), 3.07-2.92 (m, 1H), 2.85-2.67 (m, 3H), 2.63-2.54 (m, 1H), 2.33-2.21 (m, 1H), 2.08-1.96 (m, 1H), 0.93-0.85 (m, 3H).

### Example 26

### 3-(2-Chloro-3-(4-(1-methyl-2-oxo-1,2-dihydropyridin-3-yl)-4,7-diazaspiro[2.5]octan-7-yl)phenyl) piperidine-2,6-dione (Compound II-27)

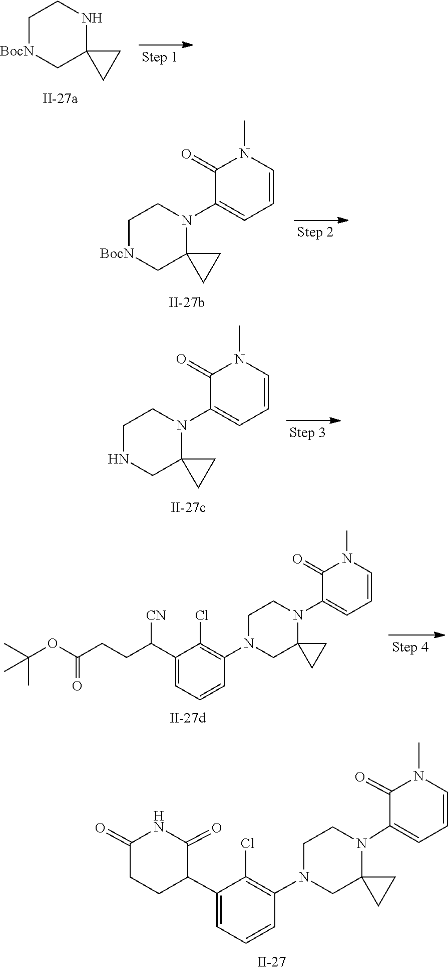

### Step 1

### tert-Butyl 4-(1-methyl-2-oxo-1,2-dihydropyridin-3-yl)-4,7-diazaspiro[2.5]octane-7-carboxylate (II-27b)

To a mixture of II-27a (420 mg, 2.0 mmol) and 1,4-dioxane (10 mL) were added 3-bromo-1-methylpyridin-2 (1H)-one (555.7 mg, 3.0 mmol), Pd₂ (dba)₃ (181.3 mg, 0.2 mmol), RuPhos (92.4 mg, 0.2 mmol) and sodium tert-butoxide (570.2 mg, 5.9 mmol), and the reaction mixture was stirred at 100° C. for 14 hours. The mixture was concentrated under reduced pressure, and the residue was purified by silica gel column chromatography (dichloromethane/methanol=100/1 to 35/1 v/v) to give the target product II-27b (400 mg, 63%).

MS m/z (ESI): 320.2 [M+H⁺]

### Step 2

### 1-Methyl-3-(4,7-diazaspiro[2.5]octan-4-yl)pyridin-2 (1H)-one (II-27c)

A mixture of II-27b (400 mg, 1.3 mmol) and a solution of hydrogen chloride in 1,4-dioxane (4 M, 10 mL) was stirred at room temperature for 1 hour. The mixture was concentrated under reduced pressure to give the target product II-27c as a hydrochloride salt (380 mg, crude product). The product was used directly in the next step without further purification.

MS m/z (ESI): 220.1 [M+H⁺]

### Step 3

### tert-Butyl 4-(2-chloro-3-(4-(1-methyl-2-oxo-1,2-dihydropyridin-3-yl)-4,7-diazaspiro[2.5]octan-7-yl)phenyl)-4-cyanobutanoate (II-27d)

II-27d was synthesized according to the procedure of Step 3 for Example 22, but II-27c was used instead of II-23c.

MS m/z (ESI): 497.2 [M+H⁺]

### Step 4

### 3-(2-Chloro-3-(4-(1-methyl-2-oxo-1,2-dihydropyridin-3-yl)-4,7-diazaspiro[2.5]octan-7-yl)phenyl) piperidine-2,6-dione (II-27)

II-27 was synthesized according to the procedure of Step 6 for Example 3, but II-27d was used instead of II-2g.

MS m/z (ESI): 441.2 [M+H⁺]

¹H NMR (400 MHz, DMSO-d₆) δ 10.86 (s, 1H), 7.28-7.21 (m, 2H), 7.08 (d, J=8.0 Hz, 1H), 7.04 (dd, J=7.3, 1.3 Hz, 1H), 6.99 (d, J=7.1 Hz, 1H), 6.17 (t, J=7.0 Hz, 1H), 4.24 (dd, J=12.1, 5.0 Hz, 1H), 3.41 (s, 3H), 2.88-2.70 (m, 3H), 2.61-2.54 (m, 1H), 2.32-2.20 (m, 1H), 2.04-1.93 (m, 3H), 1.02-0.93 (m, 2H), 0.71-0.59 (m, 2H).

Compounds II-31 and II-32 were synthesized according to the procedures for Example 26, but different chemicals were used instead of II-27a and 3-bromo-1-methylpyridin-2 (1H)-one.

| | | | Compound | |
|---|---|---|---|---|
| | | Compound | replacing 3-bromo- | |
| Compound | | replacing | 1-methylpyridin- | MS m/z |
| Number | Compound Structure | II-27a | 2(1H)-one | (ESI) |
| II-31 | 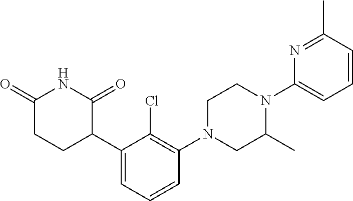 [SMILES: Cc1cccc(N2CCN(c3cccc(C4CCC(=O)NC4=O)c3Cl)CC2C)n1] | 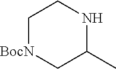 | 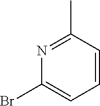 | 413.1 [M + H⁺] |
| II-32 | 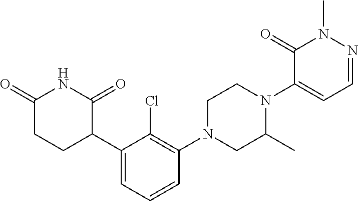 [SMILES: CC1CN(c2cccc(C3CCC(=O)NC3=O)c2Cl)CCN1c1ccnn(C)c1=O] | 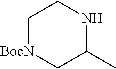 | 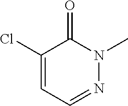 | 430.1 [M + H⁺] |

The NMR data for compound II-31 and II-32 are as follows:

| Compound | ¹H NMR |
|---|---|
| 3-(2-chloro-3-(3- | ¹H NMR (400 MHz, DMSO-d₆) δ 10.88 (s, 1H), 7.56-7.39 |
| methyl-4-(6- | (m, 1H), 7.30 (t, J = 7.8 Hz, 1H), 7.15 (d, J = 7.7 Hz, 1H), |
| methylpyridin-2- | 7.05 (d, J = 7.7 Hz, 1H), 6.75-6.47 (m, 2H), 4.62-4.56 (m, |
| yl)piperazin-1- | 1H), 4.28 (dd, J = 11.8, 4.4 Hz, 1H), 4.23-4.12 (m, 1H), |
| yl)phenyl)piperidine- | 3.33-3.17 (m, 3H), 2.96-2.70 (m, 3H), 2.62-2.53 (m, |
| 2,6-dione (II-31) | 1H), 2.42-2.22 (m, 4H), 2.06-1.95 (m, 1H), 1.35-1.24 |
| | (m, 3H). |
| 3-(2-chloro-3-(3- | ¹H NMR (400 MHz, DMSO-d₆) δ 10.87 (d, J = 2.6 Hz, 1H), |
| methyl-4-(2-methyl-3- | 7.66 (d, J = 5.0 Hz, 1H), 7.30 (t, J = 7.8 Hz, 1H), 7.15 (d, J = |
| oxo-2,3- | 7.9 Hz, 1H), 7.04 (d, J = 7.6 Hz, 1H), 6.50 (d, J = 5.0 Hz, |
| dihydropyridazin-4- | 1H), 5.08-4.96 (m, 1H), 4.27 (dd, J = 12.1, 5.0 Hz, 1H), |
| yl)piperazin-1- | 3.77-3.56 (m, 4H), 3.39-3.32 (m, 1H), 3.25 (t, J = 11.3 |
| yl)phenyl)piperidine- | Hz, 1H), 3.19-3.07 (m, 1H), 3.01-2.71 (m, 3H), 2.62- |
| 2,6-dione (II-32) | 2.53 (m, 1H), 2.36-2.22 (m, 1H), 2.05-1.94 (m, 1H), |
| | 1.32-1.18 (m, 3H). |

### Example 27

### 3-(2-Chloro-3-(4-(2-oxopyridin-1 (2H)-yl) piperazin-1-yl)phenyl) piperidine-2,6-dione (Compound II-28)

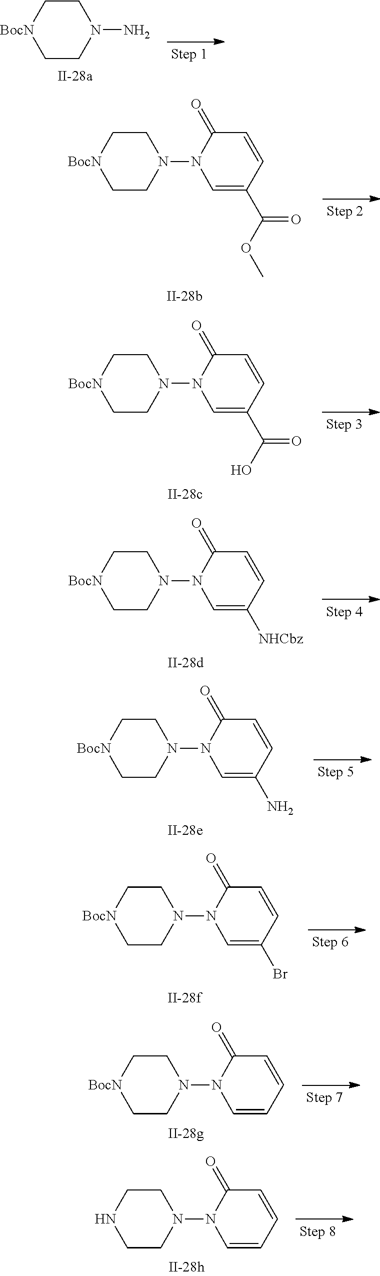

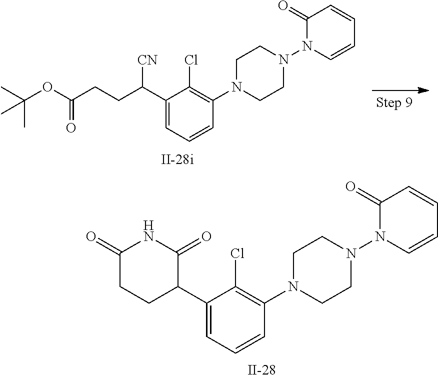

### Step 1

### tert-Butyl 4-(5-(methoxycarbonyl)-2-oxopyridin-1 (2H)-yl) piperazine-1-carboxylate (II-28b)

To a mixture of methyl 2-oxo-2H-pyran-5-carboxylate (2.3 g, 14.9 mmol) and THF (30 mL) was added II-28a (3 g, 14.9 mmol), and the reaction mixture was stirred at room temperature for 12 hours. CDI (3.6 g, 22.4 mmol) was added, and the resulting mixture was stirred at room temperature for 3 hours. The mixture was added with water and extracted with ethyl acetate. The organic phase was concentrated under reduced pressure, and the residue was purified by silica gel column chromatography (petroleum ether/ethyl acetate=100/0 to 3/1 v/v) to give the target product II-28b (3.5 g, 70%).

MS m/z (ESI): 282.1 [M+H⁺-56]

### Step 2

### 1-(4-(tert-Butoxycarbonyl) piperazin-1-yl)-6-oxo-1,6-dihydropyridine-3-carboxylic acid (II-28c)

To a solution of II-28b (1.5 g, 4.5 mmol) in THF (10 mL) was added aqueous solution of lithium hydroxide (2 M, 10 mL), and the reaction mixture was stirred at room temperature for 2 hours. The reaction was quenched with diluted hydrochloric acid (1 N) to pH=6 and extracted with ethyl acetate. The organic phase was dried over anhydrous sodium sulfate and concentrated under reduced pressure to give the target product II-28c (1.1 g, 77%).

MS m/z (ESI): 268.0 [M+H⁺-56]

### Step 3

### tert-Butyl 4-(5-(((benzyloxy) carbonyl)amino)-2-oxopyridin-1 (2H)-yl) piperazine-1-carboxylate (II-28d)

To a solution of II-28c (1 g, 3.1 mmol) and triethylamine (627 mg, 6.2 mmol) in toluene (15 mL) was added diphenylphosphoryl azide (1.7 g, 6.2 mmol), and the reaction mixture was stirred at 100° C. for 2 hours. The mixture was cooled to room temperature, added with benzyl alcohol (667 mg, 6.2 mmol), and stirred at 100° C. for another 2 hours. The reaction was quenched with saturated aqueous solution of sodium bicarbonate and extracted with ethyl acetate. The organic phase was concentrated under reduced pressure, and the residue was purified by silica gel column chromatography (petroleum ether/ethyl acetate=100/0 to ½ v/v) to give the target product II-28d (600 mg, 45%).

MS m/z (ESI): 429.3 [M+H⁺]

### Step 4

### tert-Butyl 4-(5-amino-2-oxopyridin-1 (2H)-yl) piperazine-1-carboxylate (II-28e)

To a mixture of II-28d (600 mg, 1.4 mmol) and methanol (10 mL) was added palladium on carbon (10%, containing 55% water, 60 mg), and the reaction mixture was stirred at room temperature under a hydrogen atmosphere for 3 hours. The mixture was filtered, the filter cake was washed with methanol, and the filtrate was concentrated under reduced pressure to give the target product II-28e (400 mg, 97%). The product was used directly in the next step without further purification.

MS m/z (ESI): 295.1 [M+H⁺]

### Step 5

### tert-Butyl 4-(5-bromo-2-oxopyridin-1 (2H)-yl) piperazine-1-carboxylate (II-28f)

To a solution of II-28e (400 mg, 1.4 mmol) and bromotrichloromethane (539 mg, 2.7 mmol) in dichloromethane (5 mL) were added acetic acid (82 mg, 1.4 mmol), water (5 mL) and sodium nitrite (188 mg, 2.7 mmol). The reaction mixture was stirred at room temperature overnight. The mixture was extracted with dichloromethane. The organic phase was dried over anhydrous sodium sulfate, filter, and concentrated under reduced pressure. The residue was purified by silica gel column chromatography (petroleum ether/ethyl acetate=100/0 to 5/1 v/v) to give the target product II-28f (60 mg, 12%).

MS m/z (ESI): 301.9 [M+H⁺-56]

### Step 6

### tert-Butyl 4-(2-oxopyridin-1 (2H)-yl) piperazine-1-carboxylate (II-28g)

To a solution of II-28f (50 mg, 0.14 mmol) and triethylsilane (32 mg, 0.28 mmol) in THF (5 mL) was added Pd(PPh₃)₄ (32 mg, 0.028 mmol). The reaction mixture was stirred at 80° C. for 3 hours. The mixture was concentrated under reduced pressure, and the residue was purified by silica gel column chromatography (petroleum ether/ethyl acetate=100/0 to 4/1 v/v) to give the target product II-28g (30 mg, 77%).

MS m/z (ESI): 224.1 [M+H⁺-56]

### Step 7

### 1-(Piperazin-1-yl)pyridin-2 (1H)-one (II-28h)

A mixture of II-28g (30 mg, 0.11 mmol) and a solution of hydrogen chloride in 1,4-dioxane (4 M, 3 mL) was stirred at room temperature for 2 hours. The mixture was concentrated under reduced pressure to yield the target product II-28 h as a hydrochloride salt (20 mg, 86%). The product was used directly in the next step without further purification.

MS m/z (ESI): 180.1 [M+H⁺]

### Step 8

### tert-Butyl 4-(2-chloro-3-(4-(2-oxopyridin-1 (2H)-yl) piperazin-1-yl)phenyl)-4-cyanobutanoate (II-28i)

II-28i was synthesized according to the procedure of Step 1 for Example 17, but II-28 h was used instead of tert-butyl piperazine-1-carboxylate.

MS m/z (ESI): 457.2 [M+H⁺]

### Step 9

### 3-(2-chloro-3-(4-(2-oxopyridin-1 (2H)-yl) piperazin-1-yl)phenyl) piperidine-2,6-dione (II-28)

II-28 was synthesized according to the procedure of Step 6 for Example 3, but II-28i was used instead of II-2g.

MS m/z (ESI): 401.1 [M+H⁺]

¹H NMR (400 MHz, DMSO-d₆) δ 10.87 (s, 1H), 7.64 (dd, J=6.9, 1.8 Hz, 1H), 7.43-7.38 (m, 1H), 7.29 (t, J=7.8 Hz, 1H), 7.16 (d, J=8.0 Hz, 1H), 7.01 (dd, J=11.6, 5.2 Hz, 1H), 6.41 (d, J=9.1 Hz, 1H), 6.18 (td, J=6.8, 1.5 Hz, 1H), 4.27 (dd, J=12.1, 5.1 Hz, 1H), 4.05-3.38 (m, 4H), 3.20-3.10 (m, 4H), 2.83-2.73 (m, 1H), 2.62-2.53 (m, 1H), 2.34-2.24 (m, 1H), 2.04-1.95 (m, 1H).

### Example 28

### 3-(2-Chloro-3-((S)-3-methyl-4-(2-oxo-1,2-dihydropyridin-3-yl) piperazin-1-yl)phenyl) piperidine-2,6-dione (Compound II-29)

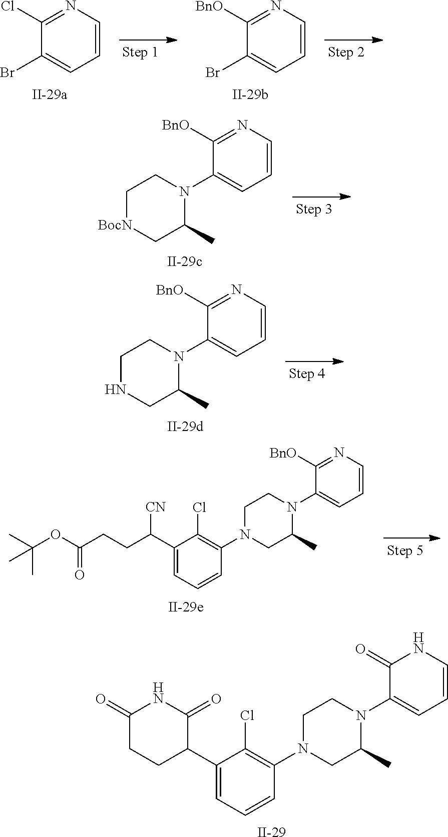

### Step 1

### 2-(Benzyloxy)-3-bromopyridine (II-29b)

To a solution of II-29a (2 g, 10.4 mmol) and benzyl alcohol (1.4 g, 12.5 mmol) in THF (25 mL) was added sodium hydride (60%, 831 mg, 20.8 mmol), and the reaction mixture was stirred at room temperature overnight. The reaction was quenched with water and extracted with ethyl acetate. The organic phase was concentrated under reduced pressure, and the residue was purified by silica gel column chromatography (petroleum ether/ethyl acetate=9/1 v/v) to give the target product II-29b (1.6 g, 58%).

MS m/z (ESI): 264.0 [M+H⁺]

### Step 2

### tert-Butyl(S)-4-(2-(benzyloxy)pyridin-3-yl)-3-methylpiperazine-1-carboxylate (II-29c)

To a solution of II-29b (1.5 g, 5.7 mmol) and II-23a (2.3 g, 11 mmol) in 1,4-dioxane (20 mL) were added Pd₂ (dba)₃ (520 mg, 0.57 mmol), BINAP (350 mg, 0.56 mmol) and sodium tert-butoxide (1.6 g, 17 mmol). The reaction mixture was stirred at 100° C. for 3 hours. The reaction was quenched with water and extracted with ethyl acetate. The organic phase was concentrated under reduced pressure, and the residue was purified by silica gel column chromatography (petroleum ether/ethyl acetate=6/1 v/v) to give the target product II-29c (400 mg, 18%).

MS m/z (ESI): 384.2 [M+H⁺]

### Step 3

### (S)-1-(2-(Benzyloxy)pyridin-3-yl)-2-methylpiperazine (II-29d)

To a solution of II-29c (380 mg, 0.88 mmol) in dichloromethane (10 mL) was added trifluoroacetic acid (2 mL), and the reaction mixture was stirred at room temperature for 2 hours. The reaction was quenched with saturated aqueous solution of sodium bicarbonate and extracted with dichloromethane. The organic phase was dried over anhydrous sodium sulfate and filtered. The filtrate was concentrated under reduced pressure to give the target product II-29d (200 mg, 71%).

MS m/z (ESI): 284.2 [M+H⁺]

### Step 4

### tert-Butyl 4-(3-((S)-4-(2-(benzyloxy)pyridin-3-yl)-3-methylpiperazin-1-yl)-2-chlorophenyl)-4-cyanobutanoate (II-29e)

To a solution of II-29d (40 mg, 0.14 mmol) and A-4 (76 mg, 0.21 mmol) in 1,4-dioxane (4 mL) were added Pd₂ (dba)₃ (13 mg, 0.014 mmol), Ruphos (7 mg, 0.015 mmol), and sodium tert-butoxide (41 mg, 0.42 mmol), and the reaction mixture was stirred at 100° C. for 6 hours. The mixture was concentrated under reduced pressure, and the residue was purified by silica gel column chromatography (petroleum ether/ethyl acetate=5/1 v/v) to give the target product II-29e (20 mg, 25%).

MS m/z (ESI): 561.3 [M+H⁺]

### Step 5

### 3-(2-Chloro-3-((S)-3-methyl-4-(2-oxo-1,2-dihydropyridin-3-yl) piperazin-1-yl)phenyl) piperidine-2,6-dione (II-29)

II-29 was synthesized according to the procedure of Step 6 for Example 3, but II-29e was used instead of II-2g.

MS m/z (ESI): 415.1 [M+H⁺]

¹H NMR (400 MHz, DMSO-d₆) δ 11.41 (s, 1H), 10.87 (s, 1H), 7.29 (t, J=7.9 Hz, 1H), 7.16 (d, J=7.7 Hz, 1H), 7.06-6.93 (m, 2H), 6.74 (d, J=7.1 Hz, 1H), 6.13 (t, J=6.7 Hz, 1H), 4.63-4.52 (m, 1H), 4.27 (dd, J=11.7, 4.7 Hz, 1H), 3.43-3.35 (m, 1H), 3.26-3.12 (m, 1H), 3.09-2.87 (m, 4H), 2.80-2.71 (m, 1H), 2.63-2.55 (m, 1H), 2.35-2.22 (m, 1H), 2.04-1.95 (m, 1H), 1.08-1.03 (m, 3H).

### Example 29

### 3-(2-Chloro-3-(4-(1-methyl-2-oxo-1,2-dihydropyridin-3-yl)-3-oxopiperazin-1-yl)phenyl) piperidine-2,6-dione (Compound II-30)

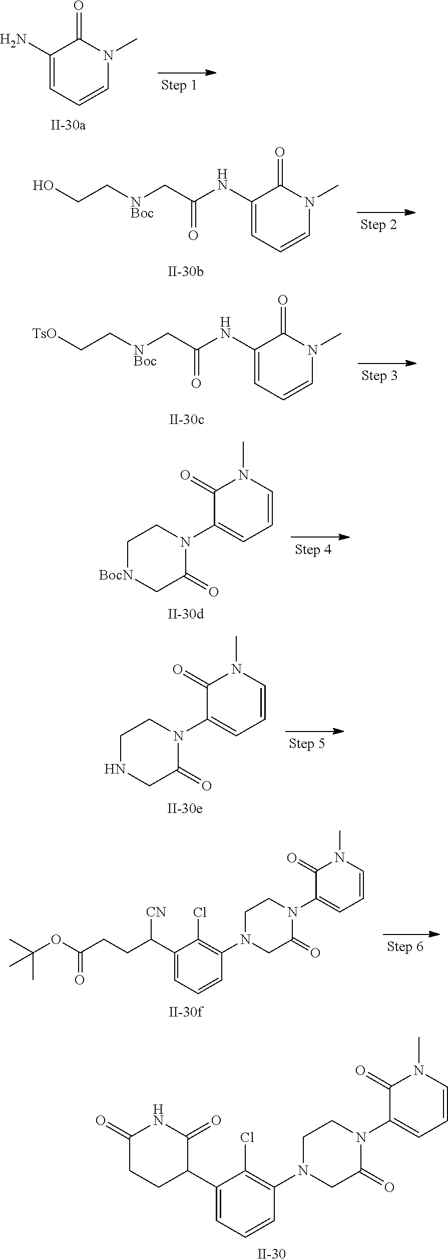

### Steps 1 to 5

### tert-Butyl 4-(2-chloro-3-(4-(1-methyl-2-oxo-1,2-dihydropyridin-3-yl)-3-oxopiperazin-1-yl)phenyl)-4-cyanobutanoate (II-30f)

II-30f was synthesized according to the procedures of Steps 2-6 for Example 12, but II-30a was used instead of II-12b.

MS m/z (ESI): 485.2 [M+H⁺]

### Step 6

### 3-(2-Chloro-3-(4-(1-methyl-2-oxo-1,2-dihydropyridin-3-yl)-3-oxopiperazin-1-yl)phenyl) piperidine-2,6-dione (II-30)

II-30 was synthesized according to the procedure of Step 6 for Example 3, but II-30f was used instead of II-2g.

MS m/z (ESI): 429.2 [M+H⁺]

¹H NMR (400 MHz, DMSO-d₆) δ 10.89 (s, 1H), 7.74 (dd, J=6.8, 1.9 Hz, 1H), 7.49 (dd, J=7.1, 1.9 Hz, 1H), 7.33 (t, J=7.8 Hz, 1H), 7.24 (dd, J=8.0, 1.4 Hz, 1H), 7.08 (dd, J=7.6, 1.3 Hz, 1H), 6.27 (t, J=6.9 Hz, 1H), 4.29 (dd, J=12.1, 5.0 Hz, 1H), 3.77 (s, 2H), 3.60 (t, J=5.2 Hz, 2H), 3.49 (s, 3H), 3.43-3.36 (m, 2H), 2.84-2.72 (m, 1H), 2.59-2.53 (m, 1H), 2.37-2.23 (m, 1H), 2.05-1.95 (m, 1H).

### Example 30

### 3-(2-Chloro-3-(1-(pyridin-3-yl) pyrrolidin-3-yl)phenyl) piperidine-2,6-dione (Compound II-34)

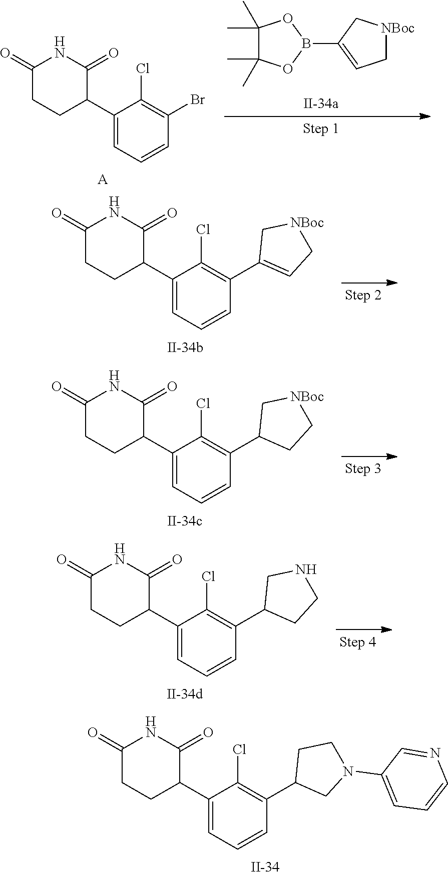

### Step 1

### tert-Butyl 3-(2-chloro-3-(2,6-dioxopiperidin-3-yl)phenyl)-2,5-dihydro-1H-pyrrole-1-carboxylate (II-34b)

To a mixture of A (2.0 g, 6.6 mmol), II-34a (2.9 g, 10.0 mmol), 1,4-dioxane (30 mL) and water (3 mL) were added DIPEA (1.7 g, 13.3 mmol) and Pd(dppf)Cl₂ (338 mg, 0.66 mmol), and the reaction mixture was stirred at 100° C. for 2 hours. The mixture was concentrated under reduced pressure, and the residue was purified by silica gel column chromatography (petroleum ether/ethyl acetate=3/2 v/v) to give the target product II-34b (2.2 g, 85%).

MS m/z (ESI): 335.0 [M+H⁺-56]

### Steps 2 to 4

### 3-(2-Chloro-3-(1-(pyridin-3-yl) pyrrolidin-3-yl)phenyl) piperidine-2,6-dione (II-34)

II-34 was synthesized according to the procedures of Steps 2-4 for Example 2, but II-34b was used instead of II-1b.

MS m/z (ESI): 370.1 [M+H⁺]

¹H NMR (400 MHz, DMSO-d₆) δ 10.90 (s, 1H), 7.99 (s, 1H), 7.92-7.83 (m, 1H), 7.37-7.28 (m, 2H), 7.24 (d, J=6.6 Hz, 1H), 7.20-7.13 (m, 1H), 6.95 (d, J=8.3 Hz, 1H), 4.31 (dd, J=12.1, 5.0 Hz, 1H), 4.01-3.89 (m, 1H), 3.76-3.68 (m, 1H), 3.52-3.32 (m, 3H), 2.84-2.71 (m, 1H), 2.62-2.55 (m, 1H), 2.44-2.26 (m, 2H), 2.19-2.08 (m, 1H), 2.05-1.95 (m, 1H).

Compound II-35 was synthesized according to the procedures for Example 30, but a different reactant was used instead of 3-iodopyridine.

| | | Compound | |
|---|---|---|---|
| Compound | | replacing 3- | MS m/z |
| Number | Compound Structure | iodopyridine | (ESI) |
| II-35 | 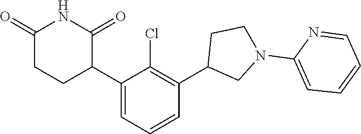 [SMILES: O=C1CCC(c2cccc(C3CCN(c4ccccn4)C3)c2Cl)C(=O)N1] | 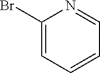 | 370.1 [M + H⁺] |

The NMR data for compound II-35 are as follows:

| Compound | ¹H NMR |
|---|---|
| 3-(2-chloro-3-(1- | ¹H NMR (400 MHz, DMSO-d₆) δ 10.89 (s, 1H), 8.15 (s, |
| (pyridin-2- | 0.5H), 8.10-8.04 (m, 1H), 7.52-7.46 (m, 1H), 7.37-7.28 |
| yl)pyrrolidin-3- | (m, 2H), 7.23 (d, J = 6.9 Hz, 1H), 6.56 (dd, J = 6.8, 5.2 Hz, |
| yl)phenyl)piperidine- | 1H), 6.48 (d, J = 8.5 Hz, 1H), 4.31 (dd, J = 12.1, 5.0 Hz, |
| 2,6-dione (II-35) | 1H), 3.96-3.82 (m, 2H), 3.62-3.38 (m, 3H), 2.84-2.72 |
| | (m, 1H), 2.62-2.54 (m, 1H), 2.42-2.25 (m, 2H), 2.21- |
| | 1.96 (m, 2H). |

### Example 31

### 3-(3-(4-(2-Aminopyridin-3-yl) piperazin-1-yl)-2-chlorophenyl) piperidine-2,6-dione (Compound II-36)

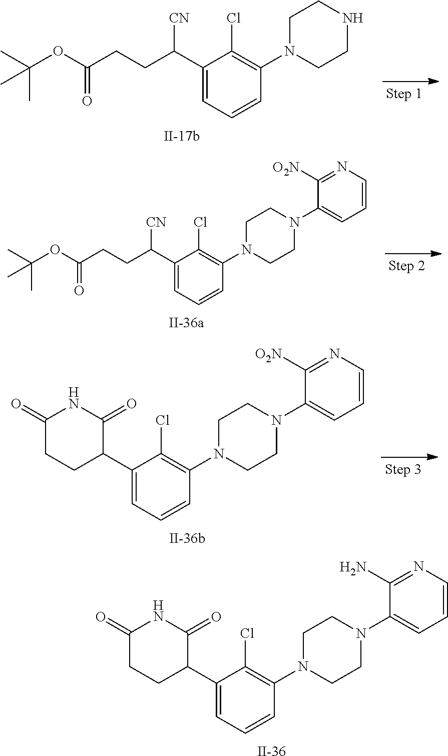

### Step 1

### tert-Butyl 4-(2-chloro-3-(4-(2-nitropyridin-3-yl) piperazin-1-yl)phenyl)-4-cyanobutanoate (II-36a)

To a solution of II-17b (100 mg, 0.28 mmol) and 3-fluoro-2-nitropyridine (59 mg, 0.42 mmol) in DMSO (5 mL) was added potassium carbonate (76 mg, 0.55 mmol), and the reaction mixture was stirred at room temperature for 2 hours. The mixture was added with water and extracted with ethyl acetate. The organic phase was concentrated under reduced pressure, and the residue was purified by silica gel column chromatography (petroleum ether/ethyl acetate=100/0 to 75/25 v/v) to give the target product II-36a (79 mg, 59%).

MS m/z (ESI): 486.2 [M+H⁺]

### Step 2

### 3-(2-Chloro-3-(4-(2-nitropyridin-3-yl) piperazin-1-yl)phenyl) piperidine-2,6-dione (II-36b)

II-36b was synthesized according to the procedure of Step 6 for Example 3, but II-36a was used instead of II-2g.

MS m/z (ESI): 430.2 [M+H⁺]

### Step 3

### 3-(3-(4-(2-Aminopyridin-3-yl) piperazin-1-yl)-2-chlorophenyl) piperidine-2,6-dione (II-36)

To a solution of II-36b (56 mg, 0.13 mmol) and ammonium chloride (35 mg, 0.65 mmol) in THF (3 mL) were added zinc powder (42 mg, 0.65 mmol) and water (3 mL), and the reaction mixture was stirred at 60° C. for 1 hour. The mixture was filtered, the filtrate was concentrated under reduced pressure, and the residue was purified by prep-HPLC to give the target product II-36 as a formate (16.5 mg, 31%).

MS m/z (ESI): 400.2 [M+H⁺]

¹H NMR (400 MHz, DMSO-d₆) δ 10.88 (s, 1H), 8.14 (s, 0.5H), 7.69 (dd, J=4.9, 1.5 Hz, 1H), 7.31 (t, J=7.8 Hz, 1H), 7.24 (dd, J=7.6, 1.4 Hz, 1H), 7.17 (dd, J=8.0, 1.3 Hz, 1H), 7.03 (dd, J=7.6, 1.2 Hz, 1H), 6.57 (dd, J=7.5, 4.9 Hz, 1H), 5.59 (s, 2H), 4.27 (dd, J=12.0, 5.0 Hz, 1H), 3.22-3.07 (m, 4H), 3.05-2.93 (m, 4H), 2.83-2.70 (m, 1H), 2.61-2.54 (m, 1H), 2.35-2.22 (m, 1H), 2.06-1.94 (m, 1H).

### Example 32

### 3-(2-Chloro-3-((S)-3-methyl-4-(1-methyl-6-oxo-1,6-dihydropyrimidin-5-yl) piperazin-1-yl)phenyl) piperidine-2,6-dione (Compound II-37)

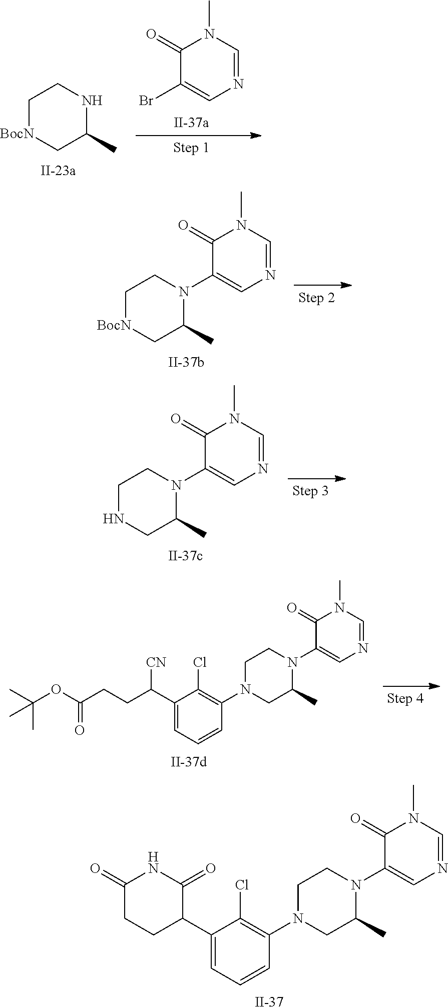

### Steps 1 to 2

### (S)-3-Methyl-5-(2-methylpiperazin-1-yl)pyrimidin-4 (3H)-one (II-37c)

II-37c was synthesized according to the procedures of Steps 1-2 for Example 4, but II-23a was used instead of tert-butyl piperazine-1-carboxylate, II-37a was used instead of II-2b.

MS m/z (ESI): 209.1 [M+H⁺]

### Steps 3 to 4

### 3-(2-Chloro-3-((S)-3-methyl-4-(1-methyl-6-oxo-1,6-dihydropyrimidin-5-yl) piperazin-1-yl)phenyl) piperidine-2,6-dione (II-37)

II-37 was synthesized according to the procedures of Steps 3-4 for Example 22, but II-37c was used instead of II-23c.

MS m/z (ESI): 430.2 [M+H⁺]

¹H NMR (400 MHz, DMSO-d₆) δ 10.87 (s, 1H), 8.16 (s, 1H), 7.42 (s, 1H), 7.29 (t, J=7.8 Hz, 1H), 7.16 (d, J=7.7 Hz, 1H), 7.03 (d, J=7.5 Hz, 1H), 4.47-4.37 (m, 1H), 4.27 (dd, J=12.0, 5.0 Hz, 1H), 3.42 (s, 3H), 3.38-3.33 (m, 1H), 3.17 (t, J=10.4 Hz, 1H), 3.10-2.88 (m, 4H), 2.82-2.72 (m, 1H), 2.59-2.53 (m, 1H), 2.35-2.22 (m, 1H), 2.04-1.95 (m, 1H), 1.13-1.04 (m, 3H).

### Example 33

### 2-(4-(2-Chloro-3-(2,6-dioxopiperidin-3-yl)phenyl) piperazin-1-yl)benzamide (Compound II-38)

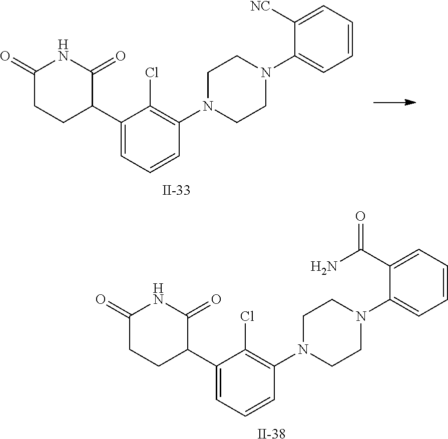

A mixture of II-33 (4 mg, 0.01 mmol) and concentrated sulfuric acid (0.5 mL) was stirred at room temperature overnight. The mixture was diluted by dropping into water, and the resulting mixture was purified by prep-HPLC to give the target product II-38 (2.3 mg, 60%).

MS m/z (ESI): 427.1 [M+H⁺]

¹H NMR (400 MHz, DMSO-d₆) δ 10.88 (s, 1H), 8.51 (s, 1H), 7.76-7.69 (m, 1H), 7.56-7.49 (m, 1H), 7.47-7.42 (m, 1H), 7.35-7.24 (m, 2H), 7.21-7.12 (m, 2H), 7.04 (d, J=7.3 Hz, 1H), 4.28 (dd, J=11.9, 5.0 Hz, 1H), 3.23-3.06 (m, 8H), 2.83-2.72 (m, 1H), 2.62-2.54 (m, 1H), 2.33-2.22 (m, 1H), 2.04-1.94 (m, 1H).

### Example 34

### 3-(2-Chloro-3-(4-(2-(hydroxymethyl)phenyl) piperazin-1-yl)phenyl) piperidine-2,6-dione (Compound II-39)

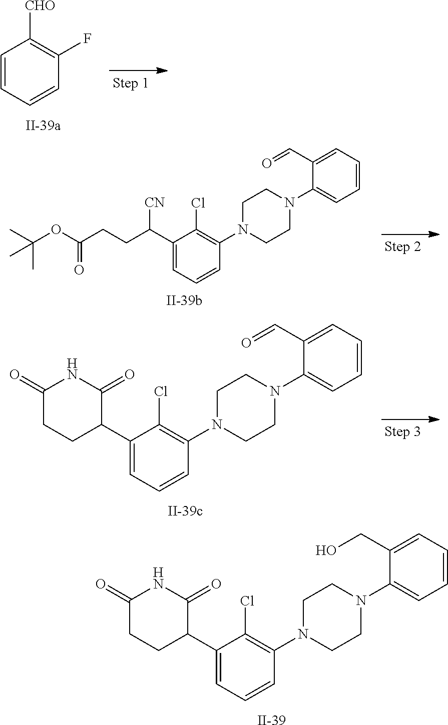

### Step 1

### tert-Butyl 4-(2-chloro-3-(4-(2-formylphenyl) piperazin-1-yl)phenyl)-4-cyanobutanoate (II-39b)

To a solution of II-39a (255 mg, 2.1 mmol) and II-17b (510 mg, 1.4 mmol) in DMSO (9 mL) was added potassium carbonate (590 mg, 4.3 mmol), and the reaction mixture was stirred at 130° C. overnight. The reaction was quenched with water and extracted with ethyl acetate. The organic phase was dried over anhydrous sodium sulfate, concentrated under reduced pressure, and purified by silica gel column chromatography (petroleum ether/ethyl acetate=90/10 v/v) to give the target product II-39b (49 mg, 8%).

MS m/z (ESI): 468.2 [M+H⁺]

### Step 2

### 2-(4-(2-Chloro-3-(2,6-dioxopiperidin-3-yl)phenyl) piperazin-1-yl)benzaldehyde (II-39c)

To a solution of II-39b (49 mg, 0.11 mmol) in acetic acid (2.5 mL) was added concentrated sulfuric acid (0.25 mL), and the reaction mixture was stirred at 70° C. overnight. The mixture was concentrated under reduced pressure. The residue was added with water and ethyl acetate. The organic phase was dried over anhydrous sodium sulfate, filter, and concentrated under reduced pressure to give the target product II-39c (33 mg, 76%). The product was used directly in the next step without further purification.

MS m/z (ESI): 412.1 [M+H⁺]

### Step 3

### 3-(2-Chloro-3-(4-(2-(hydroxymethyl)phenyl) piperazin-1-yl)phenyl) piperidine-2,6-dione (II-39)

To a solution of II-39c (33 mg, 0.08 mmol) in THF (1 mL) was added sodium borohydride (16 mg, 0.42 mmol), and the reaction mixture was stirred at room temperature for 2 hours. The reaction was quenched with diluted hydrochloric acid (1 N) and extracted with ethyl acetate. The organic phase was dried over anhydrous sodium sulfate, filtered, and concentrated under reduced pressure. The residue was purified by prep-HPLC to give the target product II-39 (6.7 mg, 20%).

MS m/z (ESI): 414.1 [M+H⁺]

¹H NMR (400 MHz, DMSO-d₆) δ 10.88 (s, 1H), 7.47 (d, J=7.5 Hz, 1H), 7.30 (t, J=7.8 Hz, 1H), 7.26-7.17 (m, 2H), 7.16-7.00 (m, 3H), 5.08 (s, 1H), 4.60 (s, 2H), 4.27 (dd, J=12.0, 5.0 Hz, 1H), 3.19-2.98 (m, 8H), 2.82-2.72 (m, 1H), 2.58-2.53 (m, 1H), 2.36-2.22 (m, 1H), 2.06-1.95 (m, 1H).

### Example 35

### 3-(2-Chloro-3-(6-(pyridin-2-yl)-2,6-diazaspiro[3.4]octan-2-yl)phenyl) piperidine-2,6-dione (Compound II-40)

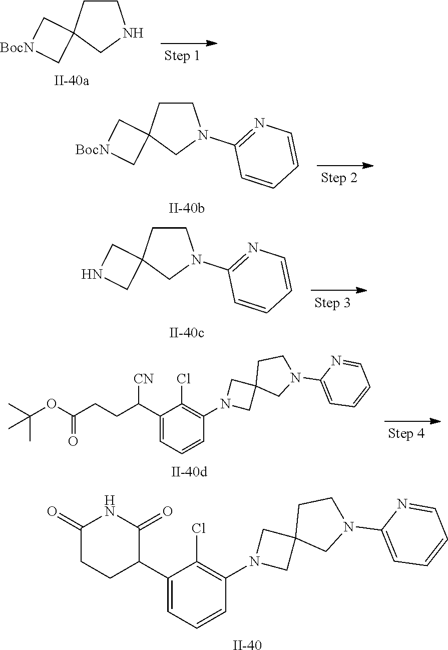

### Step 1

### tert-Butyl 6-(pyridin-2-yl)-2,6-diazaspiro[3.4]octane-2-carboxylate (II-40b)

To a solution of 2-bromopyridine (1.2 g, 7.6 mmol) and II-40a (1 g, 4.7 mmol) in toluene (20 mL) were added cesium carbonate (4.6 g, 14 mmol), Pd₂ (dba)₃ (500 mg, 0.55 mmol) and BINAP (600 mg, 0.96 mmol). The reaction mixture was stirred at 110° C. for 8 hours. The mixture was concentrated under reduced pressure, and the residue was purified by silica gel column chromatography (petroleum ether/ethyl acetate=80/20 v/v) to give the target product II-40b (72 mg, 5%).

MS m/z (ESI): 290.3 [M+H⁺]

### Step 2

### 6-(Pyridin-2-yl)-2,6-diazaspiro[3.4]octane (II-40c)

II-40b (72 mg, 0.249 mmol) was dissolved in a solution of hydrogen chloride in 1,4-dioxane (4 M, 5 mL, 20 mmol), and the resulting mixture was stirred at room temperature for 1 hour. The mixture was concentrated under reduced pressure to give the target product II-40c as a hydrochloride salt (61 mg, 94%). The product was used directly in the next step without further purification.

MS m/z (ESI): 190.1 [M+H⁺]

### Step 3

### tert-Butyl 4-(2-chloro-3-(6-(pyridin-2-yl)-2,6-diazaspiro[3.4]octan-2-yl)phenyl)-4-cyanobutanoate (II-40d)

To a solution of II-40c (53 mg, 0.20 mmol) and A-4 (95 mg, 0.27 mmol) in 1,4-dioxane (4 mL) were added cesium carbonate (200 mg, 0.61 mmol), Pd₂ (dba)₃ (19 mg, 0.021 mmol) and Xantphos (24 mg, 0.042 mmol), and the reaction mixture was stirred at 100° C. for 7 hours. The mixture was concentrated under reduced pressure, and the residue was purified by silica gel column chromatography (petroleum ether/ethyl acetate=74/26 v/v) to give the target product II-40d (54 mg, 57%).

MS m/z (ESI): 467.3 [M+H⁺]

### Step 4

### 3-(2-Chloro-3-(6-(pyridin-2-yl)-2,6-diazaspiro[3.4]octan-2-yl)phenyl) piperidine-2,6-dione (II-40)

To a solution of II-40d (54 mg, 0.12 mmol) in acetic acid (2 mL) was added concentrated sulfuric acid (0.2 mL), and the reaction mixture was stirred at 70° C. for 4 hours. The mixture was concentrated under reduced pressure, and the residue was purified by prep-HPLC to give the target product II-40 (2.5 mg, 5%).

MS m/z (ESI): 411.1 [M+H⁺]

¹H NMR (400 MHz, DMSO-d₆) δ 10.85 (s, 1H), 8.06-7.98 (m, 1H), 7.75-7.62 (m, 1H), 7.16 (t, J=7.9 Hz, 1H), 6.80-6.64 (m, 3H), 6.58 (d, J=7.1 Hz, 1H), 4.18 (dd, J=11.8, 5.0 Hz, 1H), 4.06-3.97 (m, 4H), 3.69 (s, 2H), 3.53 (t, J=6.8 Hz, 2H), 2.78-2.66 (m, 1H), 2.48-2.44 (m, 1H), 2.34-2.18 (m, 3H), 2.01-1.93 (m, 1H).

### Example 36

### 3-(2-Chloro-3-(1-(1-methyl-2-oxo-1,2-dihydropyridin-3-yl) azetidin-3-yl)phenyl) piperidine-2,6-dione (Compound II-41)

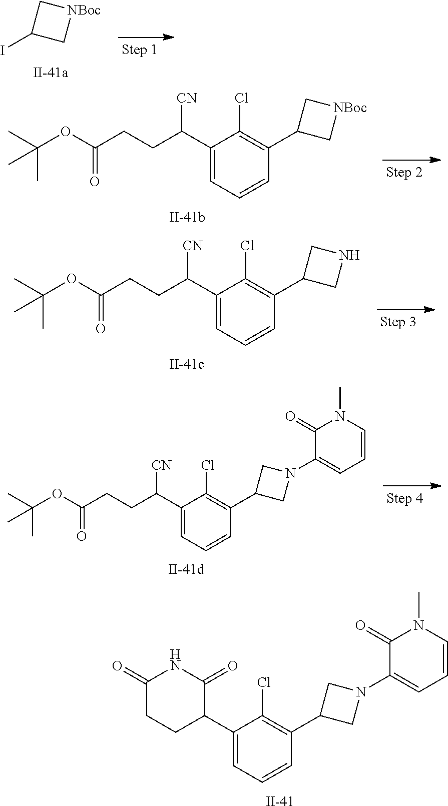

### Step 1

### tert-Butyl 3-(3-(4-(tert-butoxy)-1-cyano-4-oxobutyl)-2-chlorophenyl) azetidine-1-carboxylate (II-41b)

To a mixture of zinc powder (1.1 g, 16.8 mmol) and anhydrous DMA (20 mL) was added 1,2-dibromoethane (29 μL, 0.34 mmol), the reaction mixture was heated to reflux and stirred for 10 minutes. After cooling to room temperature, the mixture was added with trimethylsilyl chloride (21 μL, 0.17 mmol) and II-41a (500 mg, 1.77 mmol). The reaction mixture was stirred at room temperature for 1 hour. The mixture was filtered, and the filtrate was added with A-4 (600 mg, 1.7 mmol), copper (I) iodide (64 mg, 0.34 mmol) and Pd(dppf)Cl₂ (124.4 mg, 0.17 mmol). The reaction mixture was stirred at 70° C. for 6 hours. After cooling to room temperature, the mixture was diluted with water and extracted with ethyl acetate. The organic phase was dried over anhydrous sodium sulfate and filtered. The filtrate was concentrated under reduced pressure, and the residue was purified by silica gel column chromatography (petroleum ether/ethyl acetate=10/1 to 5/1 v/v) to give the target product II-41b (124 mg, 17%).

MS m/z (ESI): 323.2 [M+H⁺-112]

### Step 2

### tert-Butyl 4-(3-(azetidin-3-yl)-2-chlorophenyl)-4-cyanobutanoate (II-41c)

To a solution of II-41b (124 mg, 0.29 mmol) in dichloromethane (5 mL) was added trifluoroacetic acid (0.5 mL), and the reaction mixture was stirred at room temperature for 1 hour. The reaction was quenched with saturated aqueous solution of sodium bicarbonate and extracted with dichloromethane. The organic phase was dried over anhydrous sodium sulfate and filtered. The filtrate was concentrated under reduced pressure to give the target product II-41c (86 mg, 90%).

MS m/z (ESI): 335.1 [M+H⁺]

### Step 3

### tert-Butyl 4-(2-chloro-3-(1-(1-methyl-2-oxo-1,2-dihydropyridin-3-yl) azetidin-3-yl)phenyl)-4-cyanobutanoate (II-41d)

To a solution of II-41c (86 mg, 0.26 mmol) in 1,4-dioxane (10 mL) were added 3-bromo-1-methylpyridin-2 (1H)-one (72.2 mg, 0.37 mmol), RuPhos-Pd-G3 (21.8 mg, 0.026 mmol) and sodium tert-butoxide (74.9 mg, 0.78 mmol), and the reaction mixture was stirred at 100° C. for 2 hours. The mixture was cooled to room temperature, concentrated under reduced pressure, and purified by silica gel column chromatography (dichloromethane/methanol=50/1 to 35/1 v/v) to give the target product II-41d (60 mg, 52%).

MS m/z (ESI): 442.2 [M+H⁺]

### Step 4

### 3-(2-Chloro-3-(1-(1-methyl-2-oxo-1,2-dihydropyridin-3-yl) azetidin-3-yl)phenyl) piperidine-2,6-dione (II-41)

To a mixture of II-41d (60 mg, 0.136 mmol) and acetic acid (2 mL) was added concentrated sulfuric acid (0.2 mL), and the reaction mixture was stirred at 80° C. for 2 hours. The mixture was concentrated under reduced pressure, and the residue was purified by prep-HPLC to give the target product II-41 (formate, 5.0 mg, 10%).

MS m/z (ESI): 386.1 [M+H⁺]

¹H NMR (400 MHz, DMSO-d₆) δ 10.89 (s, 1H), 8.13 (s, 0.5H), 7.46 (d, J=7.4 Hz, 1H), 7.34 (t, J=7.6 Hz, 1H), 7.24 (d, J=7.4 Hz, 1H), 7.08 (d, J=6.3 Hz, 1H), 6.27 (d, J=6.7 Hz, 1H), 6.09 (t, J=6.9 Hz, 1H), 4.43-4.21 (m, 3H), 4.18-4.07 (m, 1H), 3.84 (t, J=6.7 Hz, 2H), 3.39 (s, 3H), 2.82-2.72 (m, 1H), 2.63-2.54 (m, 1H), 2.36-2.21 (m, 1H), 2.05-1.94 (m, 1H).

### Example 37

### 3-(2-Chloro-3-(4-(1-methyl-2-oxopiperidin-3-yl) piperazin-1-yl)phenyl) piperidine-2,6-dione (Compound II-42)

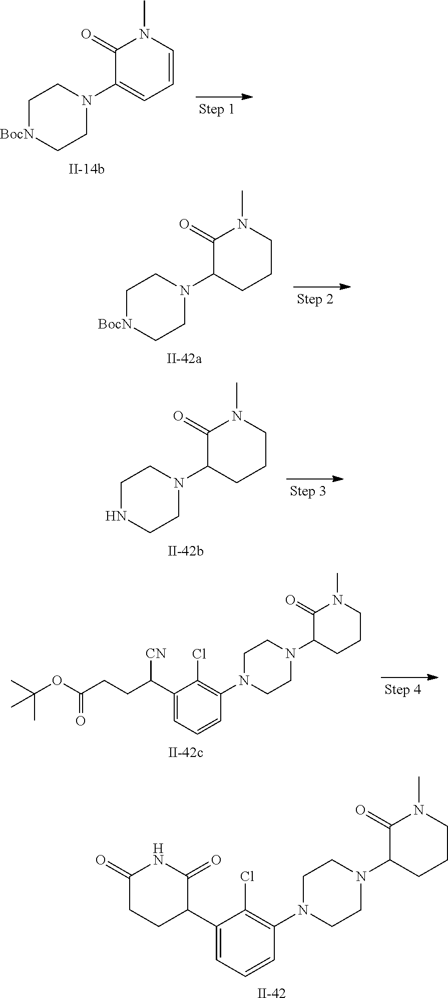

### Step 1

### tert-Butyl 4-(1-methyl-2-oxopiperidin-3-yl) piperazine-1-carboxylate (II-42a)

To a solution of II-14b (500 mg, 1.7 mmol) in methanol (20 mL) were added palladium on carbon (10%, containing 55% water, 100 mg) and raney nickel (50 mg), and the reaction mixture was stirred at 65° C. for 24 hours under a hydrogen atmosphere. The reaction mixture was filtered, and the filter cake was washed with methanol. The filtrate was concentrated, and the residue was purified by prep-HPLC to give the target product II-42a (120 mg, 23%).

MS m/z (ESI): 298.2 [M+H⁺]

### Step 2

### 1-Methyl-3-(piperazin-1-yl) piperidin-2-one (II-42b)

A mixture of II-42a (120 mg, 0.40 mmol) and a solution of HCl in 1,4-dioxane (1 M, 6 mL, 6 mmol) was stirred at room temperature for 2 hours. The mixture was concentrated under reduced pressure to give the crude product II-42b as a hydrochloride salt (85 mg, 91%).

MS m/z (ESI): 198.2 [M+H⁺]

### Steps 3 to 4

### 3-(2-Chloro-3-(4-(1-methyl-2-oxopiperidin-3-yl) piperazin-1-yl)phenyl) piperidine-2,6-dione (II-42)

II-42 was synthesized according to the procedures of Steps 5-6 for Example 3, but II-42b was used instead of II-2f and A-4 was used instead of II-2b.

MS m/z (ESI): 419.2 [M+H⁺]

¹H NMR (400 MHz, DMSO-d₆) δ 10.86 (s, 1H), 8.16 (s, 0.5H), 7.26 (t, J=7.8 Hz, 1H), 7.09 (dd, J=8.0, 1.3 Hz, 1H), 6.98 (dd, J=7.7, 1.2 Hz, 1H), 4.25 (dd, J=12.0, 5.0 Hz, 1H), 3.33-3.22 (m, 1H), 3.20-3.11 (m, 2H), 3.05-2.84 (m, 6H), 2.83-2.66 (m, 6H), 2.58-2.53 (m, 1H), 2.33-2.20 (m, 1H), 2.04-1.94 (m, 1H), 1.92-1.64 (m, 4H).

### Example 38

### 3-(2-chloro-3-((S)-3-methyl-4-(4-methyl-3-oxo-3,4-dihydropyrazin-2-yl) piperazin-1-yl)phenyl) piperidine-2,6-dione (Compound II-43)

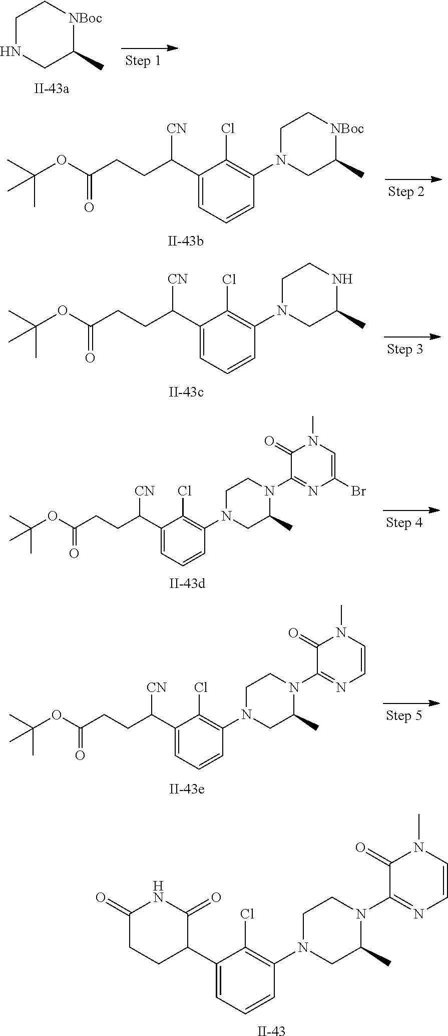

### Steps 1 to 2

### tert-Butyl 4-(2-chloro-3-((S)-3-methylpiperazin-1-yl)phenyl)-4-cyanobutanoate (II-43c)

II-43c was synthesized according to the procedures of Steps 1-2 for Example 17, but II-43a was used instead of tert-butyl piperazine-1-carboxylate.

MS m/z (ESI): 378.2 [M+H⁺]

### Step 3

### tert-Butyl 4-(3-((S)-4-(6-bromo-4-methyl-3-oxo-3,4-dihydropyrazin-2-yl)-3-methylpiperazin-1-yl)-2-chlorophenyl)-4-cyanobutanoate (II-43d)

To a stirred solution of II-43c (60 mg, 0.16 mmol) in DMF (5 mL) were added 3,5-dibromo-1-methyl-pyrazin-2-one (51 mg, 0.19 mmol) and potassium carbonate (66 mg, 0.48 mmol). The reaction mixture was stirred at 100° C. for 3 hours. Then the mixture was cooled to room temperature, diluted with water, and extracted with ethyl acetate. The organic phase was dried over sodium sulfate, filtered and concentrated under reduced pressure. The residue was purified by silica gel column chromatography (petroleum ether/ethyl acetate=80/20 to 60/40 v/v) to give the target product II-43d (41 mg, 46%).

MS m/z (ESI): 564.1 [M+H⁺]

### Step 4

### tert-Butyl 4-(2-chloro-3-((S)-3-methyl-4-(4-methyl-3-oxo-3,4-dihydropyrazin-2-yl) piperazin-1-yl)phenyl)-4-cyanobutanoate (II-43e)

To a stirred solution of II-43d (41 mg, 0.073 mmol) in THF (5 mL) were added Pd(PPh₃)₄ (25 mg, 0.022 mmol) and triethylsilane (58 μL, 0.36 mmol). The reaction mixture was stirred at 70° C. for 8 hours. Then the mixture was cooled to room temperature and concentrated under reduced pressure. The residue was purified by silica gel column chromatography (petroleum ether/ethyl acetate=60/40 to 40/60 v/v) to give the target product II-43e (33 mg, 94%)

MS m/z (ESI): 486.2 [M+H⁺]

### Step 5

### 3-(2-Chloro-3-((S)-3-methyl-4-(4-methyl-3-oxo-3,4-dihydropyrazin-2-yl) piperazin-1-yl)phenyl) piperidine-2,6-dione (II-43)

II-43 was synthesized according to the procedure of Step 6 for Example 3, but II-43e was used instead of II-2g.

MS m/z (ESI): 430.2 [M+H⁺]

¹H NMR (400 MHz, DMSO-d₆) δ 10.87 (s, 1H), 7.28 (t, J=7.8 Hz, 1H), 7.13 (d, J=7.5 Hz, 1H), 7.09 (d, J=4.3 Hz, 1H), 7.03 (d, J=7.7 Hz, 1H), 6.90 (d, J=4.3 Hz, 1H), 4.47-4.34 (m, 1H), 4.26 (dd, J=11.8, 4.8 Hz, 1H), 3.44-3.33 (m, 5H), 3.25-3.11 (m, 2H), 2.96-2.71 (m, 3H), 2.62-2.54 (m, 1H), 2.37-2.25 (m, 1H), 2.03-1.98 (m, 1H), 1.37-1.31 (m, 3H).

### Example 39

### 5-((2S)-4-(2-Chloro-3-(2,6-dioxopiperidin-3-yl)phenyl)-2-methylpiperazin-1-yl)-1-methyl-6-oxo-1,6-dihydropyridine-2-carboxamide (Compound II-44)

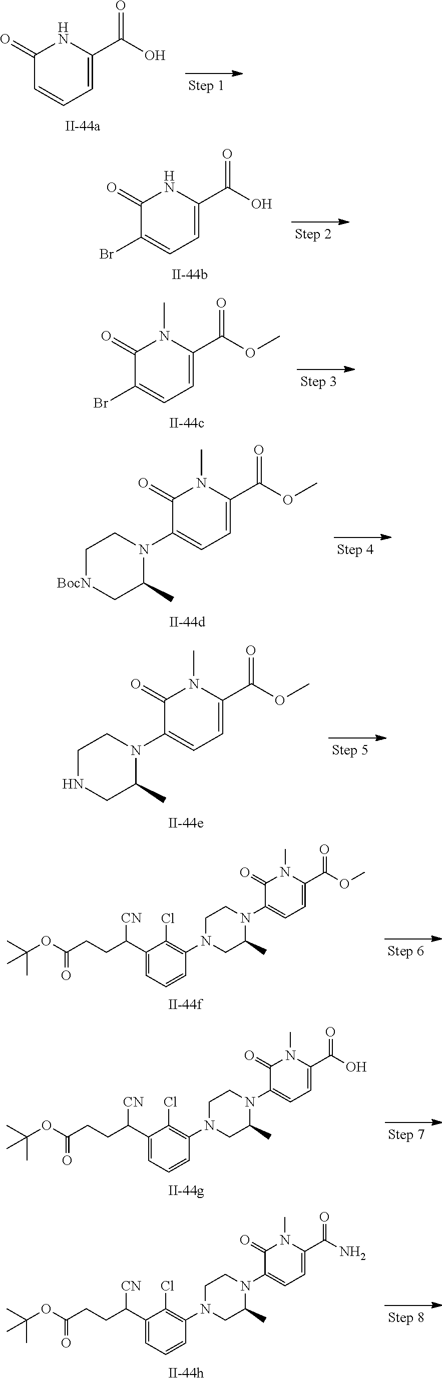

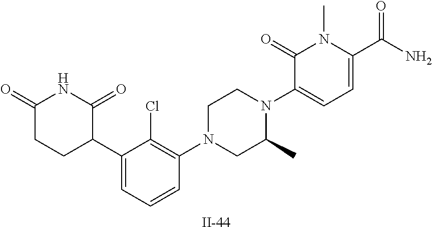

### Step 1

### 5-Bromo-6-oxo-1,6-dihydropyridine-2-carboxylic acid (II-44b)

To a solution of II-44a (10 g, 71.9 mmol) in acetic acid (160 mL) was added bromine (11.0 mL, 215 mmol) dropwise at room temperature. The reaction mixture was heated to 80° C. and stirred for 16 hours. The mixture was concentrated under reduced pressure and the residue was triturated with MTBE and filtered. The filter cake was washed with MTBE and dried to give the target product II-44b (12.9 g, 82%).

Step 2

### Methyl 5-bromo-1-methyl-6-oxo-1,6-dihydropyridine-2-carboxylate (II-44c)

To a solution of II-44b (12.9 g, 59.2 mmol) in DMF (130 mL) were added iodomethane (18.4 mL, 296 mmol) and K₂CO₃ (32.7 g, 237 mmol). The reaction mixture was heated to 50° C. and stirred for 2 hours. The mixture was poured into water and extracted with ethyl acetate. The organic phase was washed with saturated brine, dried over anhydrous sodium sulfate and filtered. The filtrate was concentrated under reduced pressure. The residue was purified by silica gel column chromatography (petroleum ether/ethyl acetate=3/1 v/v) to give the target product II-44c (6.5 g, 45%).

MS m/z (ESI): 246.0 [M+H⁺]

### Steps 3 to 4

### methyl(S)-1-methyl-5-(2-methylpiperazin-1-yl)-6-oxo-1,6-dihydropyridine-2-carboxylate (II-44e)

Compound II-44e was synthesized according to the procedures of Steps 1-2 for Example 4, but II-44c was used instead of II-2b and II-23a was used instead of tert-butyl piperazine-1-carboxylate.

MS m/z (ESI): 266.1 [M+H⁺]

### Step 5

### methyl 5-((2S)-4-(3-(4-(tert-butoxy)-1-cyano-4-oxobutyl)-2-chlorophenyl)-2-methylpiperazin-1-yl)-1-methyl-6-oxo-1,6-dihydropyridine-2-carboxylate (II-44f)

Compound II-44f was synthesized according to the procedure of Step 5 for Example 3, but II-44e was used instead of II-2f and A-4 was used instead of II-2b.

MS m/z (ESI): 543.2 [M+H⁺]

### Step 6

### 5-((2S)-4-(3-(4-(tert-butoxy)-1-cyano-4-oxobutyl)-2-chlorophenyl)-2-methylpiperazin-1-yl)-1-methyl-6-oxo-1,6-dihydropyridine-2-carboxylic acid (II-44g)

To a solution of II-44f (950 mg, 1.75 mmol) in THF (10 mL) was added aqueous lithium hydroxide solution (2 M, 1.75 mL, 3.5 mmol), and the reaction mixture was stirred at room temperature for 1 hour. Hydrochloric acid was added to make the mixture acidic (pH=6), and the resulting mixture was extracted with ethyl acetate. The organic phase was washed with saturated brine, dried over anhydrous sodium sulfate and filtered. The filtrate was concentrated under reduced pressure to give the target product II-44g (760 mg, 82%). The product was used directly in the next step without further purification.

MS m/z (ESI): 529.2 [M+H⁺]

### Step 7

### tert-Butyl 4-(3-((S)-4-(6-carbamoyl-1-methyl-2-oxo-1,2-dihydropyridin-3-yl)-3-methylpiperazin-1-yl)-2-chlorophenyl)-4-cyanobutanoate (II-44h)

To a solution of II-44g (150 mg, 0.28 mmol) and NH₄Cl (150 mg, 2.8 mmol) in DMF (5 mL) were added HATU (162 mg, 0.43 mmol) and DIPEA (183 mg, 1.4 mmol), and the reaction mixture was stirred at room temperature for 6 hours. The mixture was poured into water and extracted with ethyl acetate. The combined organic phase was washed with saturated brine, dried over anhydrous sodium sulfate and filtered. The filtrate was concentrated under reduced pressure. The residue was purified by silica gel column chromatography (dichloromethane/methanol=97/3 v/v) to give the target product II-44h (70 mg, 47%).

MS m/z (ESI): 528.2 [M+H⁺]

### Step 8

### 5-((2S)-4-(2-chloro-3-(2,6-dioxopiperidin-3-yl)phenyl)-2-methylpiperazin-1-yl)-1-methyl-6-oxo-1,6-dihydropyridine-2-carboxamide (II-44)

To a solution of II-44h (70 mg, 0.13 mmol) in acetic acid (5 mL) was added concentrated sulfuric acid (0.5 mL), and the reaction mixture was heated to 80° C. and stirred for 4 hours. The mixture was cooled to room temperature and added with saturated aqueous solution of sodium bicarbonate until the mixture became neutral. The resulting mixture was extracted with ethyl acetate. The organic phase was washed with saturated brine, dried over anhydrous sodium sulfate and filtered. The filtrate was concentrated under reduced pressure and the residue was purified by prep-HPLC to give the target product II-44 (40 mg, 63%).

MS m/z (ESI): 472.2 [M+H⁺]

¹H NMR (400 MHz, DMSO-d₆) δ 10.87 (s, 1H), 8.07 (s, 1H), 7.66 (s, 1H), 7.29 (t, J=7.8 Hz, 1H), 7.16 (d, J=7.4 Hz, 1H), 7.03 (d, J=7.4 Hz, 1H), 6.74 (d, J=7.5 Hz, 1H), 6.41 (d, J=7.6 Hz, 1H), 4.69-4.58 (m, 1H), 4.27 (dd, J=12.0, 5.0 Hz, 1H), 3.46 (s, 3H), 3.44-3.35 (m, 1H), 3.21 (t, J=10.5 Hz, 1H), 3.12-2.98 (m, 3H), 2.93 (t, J=10.6 Hz, 1H), 2.81-2.71 (m, 1H), 2.57-2.53 (m, 1H), 2.35-2.23 (m, 1H), 2.06-1.94 (m, 1H), 1.13-1.02 (m, 3H).

### Example 40

### 3-(3-((S)-4-(6-Amino-1-methyl-2-oxo-1,2-dihydropyridin-3-yl)-3-methylpiperazin-1-yl)-2-chlorophenyl) piperidine-2,6-dione (Compound II-45)

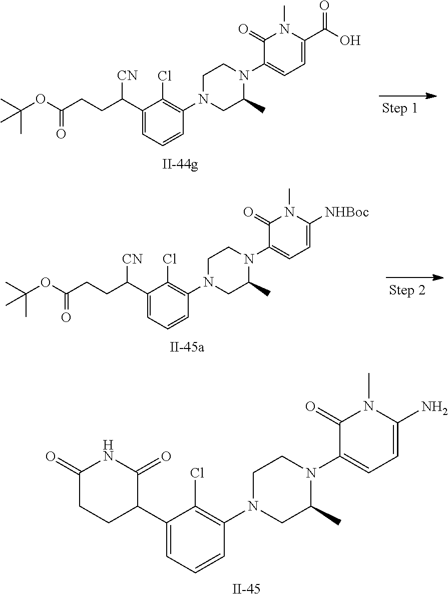

### Step 1

### tert-Butyl 4-(3-((S)-4-(6-((tert-butoxycarbonyl)amino)-1-methyl-2-oxo-1,2-dihydropyridin-3-yl)-3-methylpiperazin-1-yl)-2-chlorophenyl)-4-cyanobutanoate (II-45a)

To a solution of II-44g (160 mg, 0.3 mmol) and DPPA (166 mg, 0.6 mmol) in tert-butanol (5 mL) was added triethylamine (61 mg, 0.6 mmol) and the reaction mixture was stirred at 100° C. for 3 hours. After quenching with saturated aqueous solution of sodium bicarbonate, the mixture was extracted with ethyl acetate. The organic phase was dried over anhydrous sodium sulfate and concentrated under vacuo. The residue was purified by silica gel column chromatography (petroleum ether/ethyl acetate=1/2 v/v) to give the target product II-45a (80 mg, 44%).

MS m/z (ESI): 600.3 [M+H⁺]

### Step 2

### 3-(3-((S)-4-(6-Amino-1-methyl-2-oxo-1,2-dihydropyridin-3-yl)-3-methylpiperazin-1-yl)-2-chlorophenyl) piperidine-2,6-dione (II-45)

To a solution of II-45a (70 mg, 0.117 mmol) in acetic acid (1 mL) was added concentrated sulfuric acid (0.1 mL) and the reaction mixture was stirred at 90° C. for 2 hours. The mixture was cooled to room temperature and added with ethyl acetate. The solid was collected by filtration, purified by silica gel column chromatography (dichloromethane/methanol=20/1 v/v), and further purified by prep-HPLC to give the target product II-45 as a formate (2.6 mg, 5%).

MS m/z (ESI): 444.2 [M+H⁺]

¹H NMR (400 MHz, DMSO-d₆) δ 10.86 (s, 1H), 8.25 (m, 0.5H), 7.27 (t, J=7.8 Hz, 1H), 7.12 (d, J=7.9 Hz, 1H), 6.99 (d, J=7.6 Hz, 1H), 6.89 (d, J=8.1 Hz, 1H), 6.09 (s, 2H), 5.33 (d, J=8.0 Hz, 1H), 4.26 (dd, J=12.1, 4.8 Hz, 1H), 3.84-3.72 (m, 1H), 3.31 (s, 3H), 3.20-3.11 (m, 3H), 2.99-2.86 (m, 2H), 2.82-2.70 (m, 1H), 2.65-2.54 (m, 2H), 2.33-2.24 (m, 1H), 2.04-1.95 (m, 1H), 0.81 (d, J=6.2 Hz, 3H).

### Biology Experiments

### Example 41

### Determination of VAV1 Degradation

The test compound was dissolved in DMSO and diluted to 2 mM, followed by a 5-fold serial dilution with DMSO. Each concentration point was further diluted by 50-fold with RPMI1640 medium (Thermo Fisher, catalog number 72400-047). If the compound had a low DC₅₀ value, the starting concentration of the compound was further reduced.

Jurkat VAV1-HiBiT cells were obtained by overexpressing VAV1 fused with HiBiT at C-terminus in Jutkat E6.1 cells (Nanjing Cobioer, catalog number CBP60942). The cells were cultured in RPMI1640 complete medium [which contained 10% FBS (ExCell, catalog number FSP500)]. Cells (300,000 cells/mL) were seeded in 27 μL of complete medium in a 384-well plate, with 3 μL of compound solution added per well, and incubated at 37° C. in a 5% CO₂ incubator for 24 hours. The cell culture plate was equilibrated to room temperature, and then processed according to instruction from the Nano-GloHiBiT Lytic System kit (Promega, catalog number N3030), by adding an equal volume of Nano-Glo reagent for complete lysis. After standing at room temperature for 20 minutes, the luminescence signal was read using a microplate reader (EnVision, Perkin Elmer). The group containing 0.2% DMSO was used as a non-VAV1 degradation control and the group containing medium only was used as the background control. % degradation=1−(luminescence signal_{compound}−luminescence signal_{background})/(luminescence signal_{DMSO}−luminescence signal_{background})×100%. The compound induced VAV1 degradation curve was plotted using XLfit software (ID Business Solutions Ltd., UK) to calculate its DC₅₀ value. The experimental results are shown in Table 1.

### Example 42

### Determination of Inhibition of IL-2 Activation Induced by CD3/CD28

The test compound was dissolved in DMSO and diluted to 2 or 5 mM, followed by a 5-fold serial dilution with DMSO. Each concentration point was further diluted by 62.5-fold with RPMI1640 medium (ThermoFisher, catalog number 72400-047). If the compound's IC₅₀ value was low, the starting concentration of the compound was further reduced.

Jurkat IL-2-Luc cells (BPS Bioscience, catalog number 60481) were cultured at 37° C. in RPMI1640 medium with addition of a mixture of 10% FBS (ExCell Bio, catalog number FSP500) and 100 units/mL penicillin-streptomycin (ThermoFisher, catalog number 15140122). The cells were seeded into 384-well plates (ThermoFisher, catalog number 164610) containing 30 μL RPMI1640 medium at a density of 1×10⁶ cells/mL per well. After overnight incubation at 37° C., 5 μL of the test compound solution was added to each well, followed by addition of 5 μL of antibody mixture 30 minutes later [containing a final concentration of 10 μg/mL CD3 antibody (ThermoFisher, catalog number 16-0037-85) and 5 μg/mL CD28 antibody (ThermoFisher, catalog number 16-0289-85)]. The cells were further incubated for 6 hours at 37° C. in a 5% CO₂ incubator, then processed according to instructions from the ONE-Glo luciferase assay kit (Promega, catalog number E6120). The cell culture plate was equilibrated to room temperature, 20 μL of ONE-Glo reagent was added and mixed thoroughly, and after a 10-minute incubation at room temperature, the luminescence signal at 560 nm emission wavelength was read using a plate reader (En Vision, Perkin Elmer).

In this experiment, the 0% inhibition group was one added with a mixture of CD3 and CD28 antibodies for stimulation, but no test compound added. The 100% inhibition group was one without addition of a mixture of antibodies and the test compound. % inhibition=100-100* (luminescence signal_{compound}−luminescence signal_{100% inhibition})/(luminescence signal_{0% inhibition}−luminescence signal_{100% inhibition}). The XLfit software was used to plot the inhibition curve of the test compound on CD3/CD28 induced IL-2 activation and calculate the corresponding IC₅₀ values. The experimental results are shown in Table 1.

TABLE 1

| | | | Inhibition of CD3/CD28 |
|---|---|---|---|
| Compound | VAV1 Degradation | VAV1 Degradation | induced IL-2 activation |
| Number | DC₅₀ (nM) | Dₘₐₓ (%) | IC₅₀ (nM) |
| II-1 | 28 | 89 | 139 |
| II-2 | >2000 | | |
| II-3 | 56 | 50 | 2211 |
| II-4A | >2000 | | |
| II-4B | 573 | 70 | |
| II-5 | 63 | 44 | |
| II-6A | >2000 | | |
| II-6B | >2000 | | |
| II-7 | >2000 | | |
| II-8 | >2000 | | |
| II-9 | >2000 | | |
| II-10 | >2000 | | |
| II-11 | 150 | 42 | 955 |
| II-12 | >2000 | | |
| II-13 | >2000 | | |
| II-14 | 15 | 90 | 111 |
| II-15 | >2000 | | |
| II-16 | >2000 | | |
| II-17 | 22 | 92 | 59 |
| II-18 | 51 | 78 | 342 |
| II-19 | >2000 | | |
| II-20 | 24 | 92 | 128 |
| II-21 | 4.0 | 97 | 6.5 |
| II-22 | 50 | 73 | 322 |
| II-23 | 1.4 | 98 | 4.1 |
| II-24 | >2000 | | |
| II-25 | 3 | 98 | 5.7 |
| II-26 | >2000 | | |
| II-27 | 21 | 93 | 46 |
| II-28 | 4.4 | 98 | 6.3 |
| II-29 | 3.4 | 98 | 8.7 |
| II-30 | >2000 | | |
| II-31 | >2000 | | |
| II-32 | 64 | 52 | |
| II-33 | >2000 | | |
| II-34 | >2000 | | |
| II-35 | >2000 | | |
| II-36 | >2000 | | |
| II-37 | 5.5 | 93 | 31 |
| II-38 | >2000 | | |
| II-39 | >2000 | | |
| II-40 | >2000 | | |
| II-41 | >2000 | | |
| II-42 | 198 | 80 | 770 |
| II-43 | 44 | 80 | 197 |
| II-44 | 2.0 | 97 | 2.1 |
| II-45 | 36 | 97 | 47 |

### Example 43

### In Vivo Efficacy Study in MOG35-55-Induced EAE Model in Female C57BL/6 Mice

Female C57BL/6 mice (6-8 weeks old) were purchased from Beijing Vital River Laboratory Animal Technology Co., Ltd and were acclimatized at least one week prior to immunization. Desiccated M. tuberculosis H37Ra (BD, #231141) was suspended in incomplete Freund's Adjuvant (IFA, Merck, #F5506) to a final concentration of 8 mg/mL, and then mixed with an equal volume of a MOG35-55 peptide solution (3 mg/mL in PBS). The resulting mixture was thoroughly homogenized to an emulsion on ice using a homogenizer. Each mouse was immunized with 100 μL of the emulsion via subcutaneous injection at the back and flank (200 μL in total). Pertussis toxin (500 ng per mouse) in 200 μL of PBS was intraperitoneally administered on the day of immunization and 48 hours later. Compound II-23 was dissolved in 10% (w/v) captisol and orally administered daily at 1 mg/kg starting from the immunization day. The clinical symptom was evaluated based on the following scoring system:

EAE clinical symptoms and scoring:
0: No obvious change;
0.5: Tip of tail is limp;
1.0: Limp tail;
1.5: Limp tail and hind leg inhibition;
2: Limp tail and weakness of hind legs;
2.5: Limp tail and dragging of hind legs;
3.0: Limp tail and complete paralysis of hind legs (most common);
3.5: Limp tail and complete paralysis of hind legs;
4.0: Limp tail, complete hind leg and partial front leg paralysis;
4.5: Complete hind and partial front leg paralysis;
5.0: Moribund.

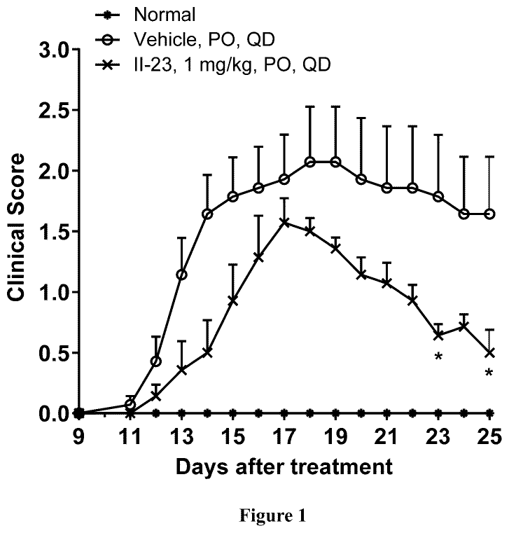

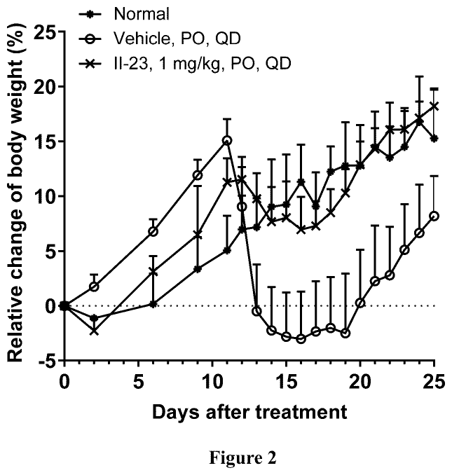

Weight loss was also monitored as an indicator of disease severity. Clinical score and body weight were recorded daily during treatment. The results showed that daily oral dosing of Compound II-23 at 1 mg/kg significantly alleviated EAE progression of mice with less clinical scores (FIG. 1) and did not have significant weight loss (FIG. 2) in the treatment group compared to those in the vehicle group.

What is claimed is:

1. A compound or its pharmaceutically acceptable salts, stable isotope derivatives and stereoisomers, wherein the compound is:

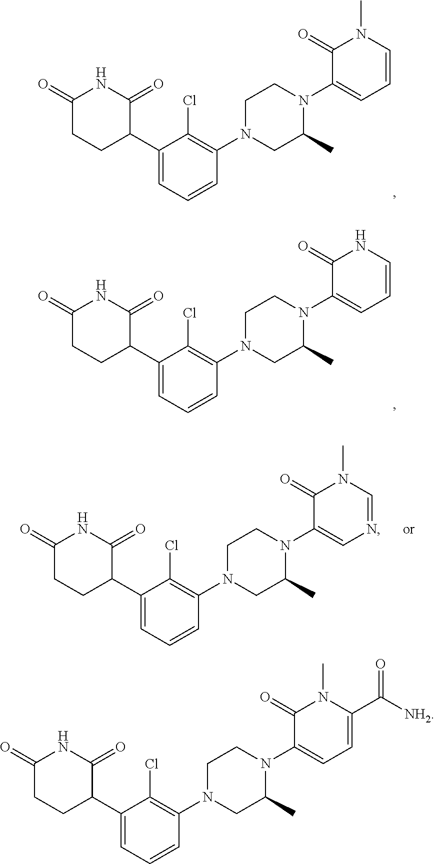 [SMILES: C[C@H]1CN(c2cccc(C3CCC(=O)NC3=O)c2Cl)CCN1c1cccn(C)c1=O ; C[C@H]1CN(c2cccc(C3CCC(=O)NC3=O)c2Cl)CCN1c1ccc[nH]c1=O ; C[C@H]1CN(c2cccc(C3CCC(=O)NC3=O)c2Cl)CCN1c1cncn(C)c1=O ; C[C@H]1CN(c2cccc(C3CCC(=O)NC3=O)c2Cl)CCN1c1ccc(C(N)=O)n(C)c1=O]

2. The compound or its pharmaceutically acceptable salts, stable isotope derivatives and stereoisomers of claim 1, wherein the compound is

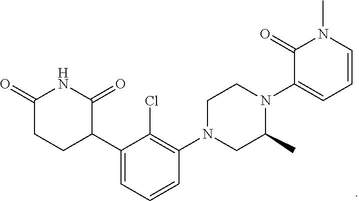 [SMILES: C[C@H]1CN(c2cccc(C3CCC(=O)NC3=O)c2Cl)CCN1c1cccn(C)c1=O]

3. The compound or its pharmaceutically acceptable salts, stable isotope derivatives and stereoisomers of claim 1, wherein the compound is

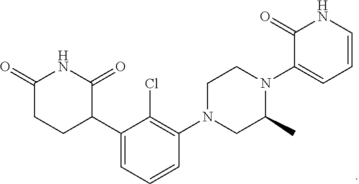 [SMILES: C[C@H]1CN(c2cccc(C3CCC(=O)NC3=O)c2Cl)CCN1c1ccc[nH]c1=O]

4. The compound or its pharmaceutically acceptable salts, stable isotope derivatives and stereoisomers of claim 1, wherein the compound is

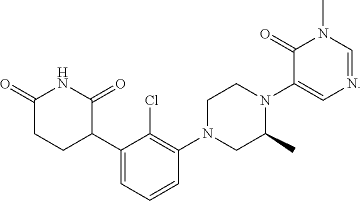 [SMILES: C[C@H]1CN(c2cccc(C3CCC(=O)NC3=O)c2Cl)CCN1c1cncn(C)c1=O]

5. The compound or its pharmaceutically acceptable salts, stable isotope derivatives and stereoisomers of claim 1, wherein the compound is

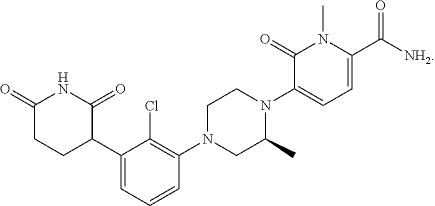 [SMILES: C[C@H]1CN(c2cccc(C3CCC(=O)NC3=O)c2Cl)CCN1c1ccc(C(N)=O)n(C)c1=O]

6. A pharmaceutical composition comprising the compound of claim 1, or its pharmaceutically acceptable salt, stable isotope derivative, stereoisomer thereof, and one or more pharmaceutically acceptable carriers or excipients.

7. A pharmaceutical composition comprising the compound of claim 2, or its pharmaceutically acceptable salt, stable isotope derivative, stereoisomer thereof, and one or more pharmaceutically acceptable carriers or excipients.

8. A pharmaceutical composition comprising the compound of claim 3, or its pharmaceutically acceptable salt, stable isotope derivative, stereoisomer thereof, and one or more pharmaceutically acceptable carriers or excipients.

9. A pharmaceutical composition comprising the compound of claim 4, or its pharmaceutically acceptable salt, stable isotope derivative, stereoisomer thereof, and one or more pharmaceutically acceptable carriers or excipients.

10. A pharmaceutical composition comprising the compound according to claim 5, or its pharmaceutically acceptable salt, stable isotope derivative, stereoisomer thereof, and one or more pharmaceutically acceptable carriers or excipients.

11. A method for treating a disease mediated by VAV1, comprising administering to a patient in need thereof a therapeutically effective amount of the compound according to claim 1, or its pharmaceutically acceptable salt, stable isotope derivative, stereoisomer thereof, wherein the disease mediated by VAV1 is an autoimmune disease or an inflammatory disease.

12. A method for treating a disease mediated by VAV1, comprising administering to a patient in need thereof a therapeutically effective amount of the compound according to claim 2, or its pharmaceutically acceptable salt, stable isotope derivative, stereoisomer thereof, wherein the disease mediated by VAV1 is an autoimmune disease or an inflammatory disease.

13. A method for treating a disease mediated by VAV1, comprising administering to a patient in need thereof a therapeutically effective amount of the compound according to claim 3, or its pharmaceutically acceptable salt, stable isotope derivative, stereoisomer thereof, wherein the disease mediated by VAV1 is an autoimmune disease or an inflammatory disease.

14. A method for treating a disease mediated by VAV1, comprising administering to a patient in need thereof a therapeutically effective amount of the compound according to claim 4, or its pharmaceutically acceptable salt, stable isotope derivative, stereoisomer thereof, wherein the disease mediated by VAV1 is an autoimmune disease or an inflammatory disease.

15. A method for treating a disease mediated by VAV1, comprising administering to a patient in need thereof a therapeutically effective amount of the compound according to claim 5, or its pharmaceutically acceptable salt, stable isotope derivative, stereoisomer thereof, wherein the disease mediated by VAV1 is an autoimmune disease or an inflammatory disease.
